SEQUENCE LISTING (2 sequences)

SEQ ID NO: 1 - DNA, 20 bp, Artificial Sequence
  Description of Artificial Sequence Synthetic primer
tagggcaaaccatctggaag

SEQ ID NO: 2 - DNA, 20 bp, Artificial Sequence
  Description of Artificial Sequence Synthetic primer
acttggagctacaggcctca

(19) United States
(12) Patent Application Publication
(10) Pub. No.: US 2013/0195798 A1
(43) Pub. Date: Aug. 1, 2013

(54) COMBINATION THERAPY FOR TREATMENT OF VIRAL INFECTIONS

(71) Applicants: United Therapeutics Corporation, Silver Spring, MD (US); The Chancellors, Masters and Scholars of the Univ, Oxford (GB)

(72) Inventors: Roger Jeffs, Chapel Hill, NC (US); S. Karl Gotzkowsky, New Orleans, LA (US); Raymond Allen Dwek, Oxford (GB); Nicole Zitmann, Oxford (GB)

(73) Assignees: The Chancellors, Masters and Scholars of the University of Oxford, Oxford (GB); United Therapeutics Corporation, Silver Spring, MD (US)

(21) Appl. No.: 13/683,036

(22) Filed: Nov. 21, 2012

Related U.S. Application Data
(62) Division of application No. 11/842,569, filed on Aug. 21, 2007, now abandoned.
(60) Provisional application No. 60/838,872, filed on Aug. 21, 2006.
(60) Provisional application No. 60/874,498, filed on Dec. 13, 2006.
(60) Provisional application No. 60/894,307, filed on Mar. 12, 2007.

(51) Int. Cl. A61K 38/21; A61K 31/45; A61K 31/7056

(52) U.S. Cl. CPC A61K 38/212; A61K 38/21; A61K 31/7056; A61K 31/45
USPC 424/85.7; 424/85.4; 514/315

(57) ABSTRACT

One can treat a viral infection by first administering at least one first antiviral compound for a first time period and then, after the end of the first time period, concurrently or subsequently administering the at least one first antiviral compound and at least one second antiviral compound for a second period. In some cases, after the end of the second period, the same at least one second antiviral compound that was administered during the second time period may be administered for a third time period without concurrent or subsequent administration of the at least one first antiviral compound.

### RELATED APPLICATIONS

The present application is a Divisional of U.S. application Ser. No. 11/842,569, filed Aug. 21, 2007, which claims priority to U.S. provisional applications Nos. 60/838,872 filed Aug. 21, 2006 to Dwek et al.; 60/874,498 filed Dec. 13, 2006 to Dwek et al. and 60/894,307 filed Mar. 12, 2007 to Jeffs et al., which are all incorporated herein by reference in their entirety.

### FIELD

The present inventions generally relate to treatment of viral infections in a mammal, including humans. More particularly, the present inventions may provide methods, kits, and compositions pertaining to combination therapies for treatment of hepatitis viral infections.

### BACKGROUND

Hepatitis C virus (HCV) is an RNA virus belonging to the Flaviviridae family. Individual isolates comprise closely related, yet heterologous populations of viral genomes. This genetic diversity may enable the virus to escape the host's immune system, leading to a high rate of chronic infection. Human diseases caused by flaviviruses include various hemorrhagic fevers, hepatitis, and encephalitis. Viruses known to cause these diseases in humans have been identified and include, for example, yellow fever virus, dengue viruses 1-4, Japanese encephalitis virus, Murray Valley encephalitis virus, Rocio virus, West Nile fever virus, St. Louis encephalitis virus, tick-borne encephalitis virus, Louping ill virus, Powassan virus, Omsk hemorrhagic fever virus, and Kyasanur forest disease virus. Therapeutic interventions, which may be effective for treatment of HCV infection, are limited in number and effectiveness. Standard treatment for HCV infection includes administration of interferon-alpha and/or ribavirin. However, the complications and limitations of interferon-alpha and/or ribavirin seriously limit the applicability of the treatment.

Hepatitis B virus, a hepadnavirus, is another causative agent of acute and chronic liver disease, including liver fibrosis, cirrhosis, inflammatory liver disease, and hepatic cancer. Although effective vaccines are available, such vaccines have no therapeutic value for those already infected with the virus.

A large number of individuals, who are infected with HCV are also infected with hepatitis B virus (HBV). The therapy for combined HBV/HCV infection is particularly challenging because the HBV and HCV viruses differ from one another in therapeutically significant ways. HBV is a DNA-containing virus, the genome of which is replicated in the infected cell using a combination of a DNA-dependent RNA polymerase and an RNA-dependent DNA polymerase (i.e., a reverse transcriptase). HCV is an RNA-containing virus, the genome of which is replicated in the cytoplasm of the infected cell using one or more types of RNA-dependent RNA polymerases. Despite the frequent concurrence of HBV infection and HCV infection, a number of compounds known to be effective for treating HBV infection are not effective against HCV. For example, lamivudine (the nucleoside analog 3TC) is useful for treating HBV infection, but is not useful for treating HCV infection. The difference in the susceptibility of HBV and HCV to antiviral agents may relate to their genetically based replicative differences.

Other hepatitis viruses that are significant agents of human disease include hepatitis A, hepatitis Delta, hepatitis E, hepatitis F, and hepatitis G. In addition, there are animal hepatitis viruses that are species specific. These include, for example, those infecting ducks, woodchucks, and mice. The availability of animal models allows the preclinical testing of antiviral compounds for each class of virus. Such animal viruses include hepadnaviruses, pestiviruses and flaviviruses such as bovine viral diarrhea virus (BVDV), classical swine fever virus, border disease virus, and hog cholera virus. However, similarly robust animal models are not available for HCV. Despite years of research, a need remains for improved therapies for treating hepatitis virus infections, and/or for supplementing currently available therapies.

### SUMMARY

In one aspect, methods are provided which include contacting a mammalian cell infected with a virus with a first compound, and at least one compound selected from a second compound and a third compound, wherein the first compound, the second compound, and the third compound are contacted in an amount effective to inhibit the virus. In some embodiments, the first compound is a compound of Formula I or Formula II, or a pharmaceutically acceptable salt thereof, or a mixture of any two or more thereof:

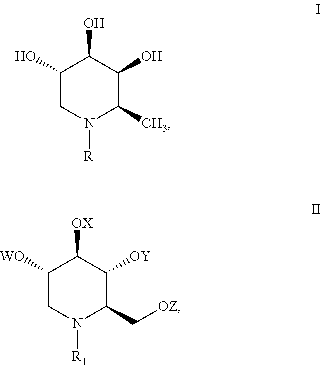

wherein R is selected from substituted or unsubstituted alkyl groups, substituted or unsubstituted cycloalkyl groups, substituted or unsubstituted heterocyclyl groups, or substituted or unsubstituted oxaalkyl groups; and where R₁ is selected from substituted or unsubstituted alkyl groups, substituted or unsubstituted cycloalkyl groups, substituted or unsubstituted aryl groups, or substituted or unsubstituted oxaalkyl groups, selected from but not limited to arylalkyl, cycloalkylalkyl, branched or straight chain alkyl groups, and oxaalkyl groups; and where W, X, Y, and Z are each independently selected from hydrogen, alkanoyl groups, aroyl groups, and haloalkanoyl groups. In some such embodiments, the second compound is selected from a nucleotide antiviral compound, a nucleoside antiviral compound, or a mixture of any two or more thereof, and the third compound is selected from an immunostimulating compound, an immunomodulating compound, or a mixture of any two or more thereof.

In some embodiments, when the first compound is a compound of Formula I, substituted or unsubstituted alkyl groups and/or substituted or unsubstituted oxaalkyl groups comprise from 1 to 16 carbon atoms, from 4 to 12 carbon atoms or from 8 to 10 carbon atoms. For example, R may be selected from, but is not limited to —(CH₂)₆OCH₃, —(CH₂)₆OCH₂CH₃, —(CH₂)₆O(CH₂)₂CH₃, —(CH₂)₆O(CH₂)₃CH₃, —(CH₂)₂O(CH₂)₅CH₃, —(CH₂)₂O(CH₂)₆CH₃, and —(CH₂)₂O(CH₂)₇CH₃.

In some embodiments, the second compound is selected from, but is not limited to purine nucleotide antiviral compounds, pyrimidine nucleotide antiviral compounds, purine nucleoside antiviral compounds, pyrimidine nucleoside antiviral compounds, and mixtures of any two or more thereof. In some embodiments, the third compound is selected from interferons, pegylated interferons, or mixtures of any two or more thereof.

In some embodiments of the methods provided, the contacting a mammalian cell step of the method comprises administering the first compound, the second compound, and the third compound to a mammal. In other embodiments, methods provide that the first compound, the second compound, and the third compound are administered to the mammal separately, sequentially, or simultaneously.

In some embodied methods, the virus belongs to the Flaviviridae or the Hepadnaviridae family of viruses. The virus may be selected from, but is not limited to hepatitis viruses such as hepatitis B virus or hepatitis C virus, or a bovine viral diarrhea virus. In such embodiments, the amount effective to inhibit the virus, is an amount effective to inhibit a hepatitis virus, a hepatitis B virus, a hepatitis C virus, or a bovine diarrhea virus.

In another aspect, kits are provided comprising a first compound, wherein the first compound is a compound of Formula I or Formula II, a pharmaceutically acceptable salt thereof, or a mixture of any two or more thereof, and least one compound selected from a second compound, as described above, and a third compound, as described above, where the first compound, the second compound, and third compound of the kit are present in an amount effective to inhibit a virus infecting a mammal. In some such embodiments, the first compound, the second compound, and the third compound of the kit form a pharmaceutical composition for simultaneous administration to the mammal. In other such embodiments, the first compound, the second compound, and the third compound of the kit are for separate or sequential administration to the mammal. In yet other embodiments, the second compound and the third compound of the kit comprise a single composition. In some such other embodiments, the first compound and the second compound of the kit comprise a single composition.

In another aspect, compositions are provided comprising a first compound, wherein the first compound is a compound of Formula I or Formula II, a pharmaceutically acceptable salt thereof, or a mixture of any two or more thereof, a second compound as described above, and a third compound as described above, where the first compound, the second compound, and the third compound are present in an amount effective to inhibit a virus. In some embodiments, the composition further comprises a pharmaceutically acceptable carrier. In such embodiments, compositions are administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral, as used herein, includes subcutaneous, intravenous, intramuscular, or intrasternal injection, or infusion techniques. Dosages and dosage forms are also provided.

Yet in another aspect, provided is a method of treating or preventing a viral infection, comprising administering to a subject in need thereof a combination that comprises a) an immunostimulating or immunomodulating agent and b) a nucleotide or a nucleoside antiviral agent, provided that said combination does not inhibit a host enzyme or does not inhibit an ion channel activity; and then after a period of time sufficient to permit the combination to enhance activity of a second administration step, administering to the subject the combination and a compound that is at least one of a host enzyme inhibitor or an ion channel inhibitor. And yet in another aspect, provided is a method of treating or preventing a viral infection, comprising decreasing a level of the viral infection in a subject in need thereof by first administering to the subject a pharmaceutical composition that does not inhibit a host enzyme or does not inhibit an ion channel activity; and then administering to the subject the composition, and a compound that is at least one of a host enzyme inhibitor or an ion channel inhibitor.

And yet in another aspect, provided is a method of treating a viral infection comprising (A) administering to a subject in need thereof at least one first antiviral agent for a first time period, wherein said at least one first antiviral agent does not inhibit host α-glucosidase; and (B) after the first time period, sequentially or concurrently administering to the subject the at least one first antiviral agent and at least one second antiviral agent for a second time period, wherein the at least one second antiviral agent inhibits host α-glucosidase.

And yet in another aspect, provided is a method of treating a viral infection comprising (A) administering to a subject in need thereof at least one first antiviral agent for a first time period, wherein said at least first antiviral agent does not comprise an iminosugar; and (B) after the first time period, sequentially or concurrently administering to the subject the at least one first antiviral agent and at least one second antiviral agent for a second time period, wherein the at least one second antiviral agent comprises an iminosugar.

And yet in another aspect, provided is a method of treating a viral infection comprising (A) administering to a subject in need thereof at least one first antiviral agent for a first time period, wherein said at least first antiviral agent does not inhibit an ion channel activity; and (B) after the first time period, sequentially or concurrently administering to the subject the at least one first antiviral agent and at least one second antiviral agent for a second time period, wherein the second antiviral agent inhibits an ion channel activity.

And yet in another aspect, provided is a method of treating a viral infection comprising (A) administering to a subject in need thereof at least one first antiviral agent for a first time period, wherein said at least one first antiviral agent does not comprise a nitrogen-containing compound of formula VIII; and (B) after the first time period, sequentially or concurrently administering to the subject the at least one first antiviral agent and at least one second antiviral agent for a second time period, wherein the at least one second antiviral agent comprises a nitrogen-containing compound of formula VIII or a pharmaceutically acceptable salt thereof:

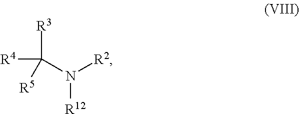

wherein R¹² is an alkyl or an oxa-substituted derivative thereof;
R² is hydrogen, R³ is carboxy, or a C₁-C₄ alkoxycarbonyl, or R² and R³, together
are

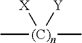

or —(CXY)ₙ—, wherein n is 3 or 4, each X, independently, is hydrogen, hydroxy, amino, carboxy, a C₁-C₄ alkylcarboxy, a C₁-C₄ alkyl, a C₁-C₄ alkoxy, a C₁-C₄ hydroxyalkyl, a C₁-C₆ acyloxy, or an aroyloxy, and each Y, independently, is hydrogen, hydroxy, amino, carboxy, a C₁-C₄ alkylcarboxy, a C₁-C₄ alkyl, a C₁-C₄ alkoxy, a C₁-C₄ hydroxyalkyl, a C₁-C₆ acyloxy, an aroyloxy, or deleted;
R⁴ is hydrogen or deleted; and
R⁵ is hydrogen, hydroxy, amino, a substituted amino, carboxy, an alkoxycarbonyl, an aminocarbonyl, an alkyl, an aryl, an aralkyl, an alkoxy, a hydroxyalkyl, an acyloxy, or an aroyloxy, or R³ and R⁵, together, form a phenyl and R⁴ is deleted.

### BRIEF DESCRIPTION OF THE DRAWINGS

FIG. 1 presents detection of ncp BVDV in the persistently infected MDBK cells by IF. After the final passage (P22) the persistently infected MDBK cells were probed for the continued presence of ncp BVDV by immunofluorescence. Data are shown for cells treated for passages 3-12 in the absence of drug (no drug), in the presence of IFN/RBV only (I/R), or with triple combinations of IFN/RBV/10 μM NB-DNJ (10 NB), IFN/RBV/100 μM 231B (100 231B), IFN/RBV/50 μM NN-DNJ (50 NN). In cells from both set 2 (all drugs removed for passages 12-22) and set 3 (IFN/RBV only removed for passages 12-22) FITC staining (green) associated with BVDV NS2-3 binding is only detected in cells not treated with drug (no drug) and in cells treated with IFN/RBV up to passage 12. Nuclei were stained with DAPI (blue).

FIG. 2 presents detection of ncp BVDV in the persistently infected MDBK cells by IF 5 passages after removal of interferon and ribavirin while maintaining NB-DNJ treatment. Data are shown for cells treated for passages 3-12 in the absence of drug (no drug), in the presence of IFN/RBV only (I/R), or with triple combinations of IFN/RBV/0.1 μM NB-DNJ, IFN/RBV/1 μM NB-DNJ, IFN/RBV/10 μM NB-DNJ.

FIG. 3 presents detection of ncp BVDV in the persistently infected MDBK cells by IF 12 passages after removal of interferon and ribavirin only while maintaining NB-DNJ treatment. Data are shown for cells treated for passages 3-12 in the absence of drug (no drug), or with triple combinations of IFN/RBV/0.1 μM NB-DNJ, IFN/RBV/1 μM NB-DNJ.

FIG. 4 (A) shows chemical structures of the iminosugar derivatives used in Example 3. NB-DNJ=N-butyl deoxynojirimycin; NN-DNJ=N-nonyl deoxynojirimycin; N7-DGJ=N7-6-deoxy-methyl-galactonojirimycin. FIG. 4(B) illustrates experimental outline of the study in Example 3. After a stable infection is established cells are cultured for three passages (nine days) in the presence of 1000 IU IFN/1 μM RBV. At passage 3 (P3), after viral RNA levels drop below the detection limit, the medium is supplemented with one of the iminosugars (IS). At the end of P8 the samples are split into three sets: Set 1 (black line), all drug regimes remain the same; Set 2 (cross-hatched line), all drugs are removed; Set 3 (grey line), only IS are continued. After P12, samples from Set 1, which had been cultured for nine passages in the presence of IFN/RBV and an IS, are split into sample Sets 1, 2a and 3a to be treated in the same manner as described above.

FIG. 5 shows viral RNA copies from supernatants harvested at P9 (left column) and P10 (right column) measured by real-time RT-PCR shown as percentage of the non-drug treated BVDV-infected control. For Set 1 all drugs are still present. For Sets 2 and 3 (E and F), P9/P10 denote one/two passage(s) after removal of all three drugs or IFN/RBV only, respectively.

FIGS. 6 (A)-(C) shows viral RNA copies from supernatants harvested after 22 passages as percentage of the non-drug treated BVDV-infected control. After initial 12 passages of the various drug treatments, all drugs were either (FIG. 6A) left on or (FIG. 6B) removed, or (FIG. 6C) cells continued to be cultured in the presence of an IS only, for a further 10 passages (30 days). Viral RNA copies at P22 were measured using real-time RT-PCR and are shown as a percentage of the non-drug treated BVDV infected control.

FIGS. 7A-7D show immunofluorescence analysis of naïve MDBK cells incubated with supernatants from treated BVDV-infected cells (set 2) at passage 10 (P10, FIG. 7A) and at passage 22 (P22, FIG. 7B) and of long-term treated BVDV-infected MDBK cells (set 2) at P22 (FIG. 7C). Cells were fixed and probed with a monoclonal antibody against BVDV NS2/3 proteins, followed by incubation with an anti-mouse FITC-conjugated secondary antibody (green). In FIG. 7D, cell nuclei were stained with DAPI (blue).

FIGS. 8A-8B show immunofluorescence analysis of treated BVDV-infected MDBK cells at passage 32 (P32). 20 passages (60 days) after removal of (FIG. 8A) all drugs or (FIG. 8B) after removing IFN/RBV (iminosugar only maintenance), cells were fixed and probed with a monoclonal antibody against BVDV NS2/3 proteins, followed by incubation with an anti-mouse FITC-conjugated secondary antibody (green). Cell nuclei were stained with DAPI (blue).

### DETAILED DESCRIPTION

Unless otherwise specified “a” or “an” means one or more.

The following definitions are used throughout:

“231B” or “N7-DGJ” refers to N-(7-oxa-nonyl)-1,5-dideoxy-1,5-imino-D-galactitol also known as 1-(6-ethoxy-hexyl)-2-methyl-piperidine-3,4,5-triol.

“BVDV” refers to bovine viral diarrhea virus.

“HBV” refers to hepatitis B virus.

“HCV” refers to hepatitis C virus.

“HPMPC” refers to S-1-3-hydroxy-2-phosphonylmethoxypropyl cytosine.

“IFN” refers to interferon.

“IF” refers to immunofluorescence.

“IU” refers to international units.

“MDBK” refers to Madine-Darby bovine kidney cells.

“MOI” refers to multiplicity of infection.

“Ncp” refers to non-cytopathic.

“NB-DNJ” refers to N-butyl deoxynojirimycin, also known as ZAVESCA® or miglustat.

“NN-DNJ” refers to N-nonyl deoxynojirimycin.

“Pfu” refers to plaque forming units.

“RBV” refers to ribavirin.

“RT” refers to reverse transcription.

“Rt-PCR” refers to reverse transcription polymerase chain reaction.

“DAPI” refers to 4′,6′-Diamidino-2-phenylindole.

In general, “substituted” refers to a functional group, as defined below, in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. In some embodiments, substituted groups have 1, 2, 3, 4, 5, or 6 substituents. Examples of substituent groups include, but are not limited to: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; ethers; urethanes; oximes; hydroxylamines; alkoxyamines; thiols; alkyl, alkenyl, alkynyl, aryl, aralkyl, heterocyclyl and heterocyclylalkyl sulfide groups; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; and nitriles.

Substituted ring groups, such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with alkyl, alkenyl, and alkynyl groups as defined below.

Alkyl groups may include straight chain and branched alkyl groups and cycloalkyl groups. Thus, alkyl groups may have from 1 to about 20 carbon atoms in some embodiments, from 1 to 12 or 1 to 8 carbon atoms in other embodiments, and from 4 to 10 carbon atoms, in yet other embodiments. Examples of straight chain alkyl groups include, but are not limited to, those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, isopentyl, and 2,2-dimethylpropyl groups. Alkyl groups may be substituted or unsubstituted. Representative substituted alkyl groups may be substituted one or more times with any of the groups listed above, for example, amino, oxo, hydroxy, cyano, carboxy, nitro, thio, alkoxy, and F, Cl, Br, I groups.

Cycloalkyl groups include cyclic alkyl groups, such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include mono-, bicyclic and polycyclic ring systems, such as, for example bridged cycloalkyl groups as described below, and fused rings, such as, but not limited to, decalinyl, and the like. Cycloalkyl groups may be substituted or unsubstituted. Substituted cycloalkyl groups may be substituted one or more times with non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with any of the groups listed above, for example, methyl, amino, hydroxy, cyano, carboxy, nitro, thio, alkoxy, and F, Cl, Br, I groups.

Alkenyl groups may include straight and branched chain alkyl and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 10 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Alkenyl groups may be substituted or unsubstituted.

Alkynyl groups may include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 10 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others. Alkynyl groups may be substituted or unsubstituted.

Aryl groups are cyclic aromatic hydrocarbons that do not contain heteroatoms. Aryl groups may include monocyclic, bicyclic and polycyclic ring systems. In some embodiments, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Aryl groups may be substituted or unsubstituted. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with groups such as those listed above.

Heterocyclyl groups include aromatic (also referred to as heteroaryl) and non-aromatic ring compounds containing 3 or more ring members, of which one or more is a heteroatom, such as, but not limited to, N, O, and S. In some embodiments, the heterocyclyl group contains 1, 2, 3, or 4 heteroatoms. In some embodiments, heterocyclyl groups include 3 to 20 ring members, whereas other such groups have 3 to 6, 10, 12, or 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]-dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups.” Heterocyclyl groups may be substituted or unsubstituted. Heterocyclyl groups include, but are not limited to, pyrrolidinyl, pyrrolinyl, imidazolyl, imidazolinyl, imidazolidinyl, piperidinyl, piperazinyl, morpholinyl, pyrrolyl, pyrazolyl, pyrazolidinyl, tetrahydropyranyl, thiomorpholinyl, pyranyl, triazolyl, tetrazolyl, furanyl, tetrahydrofuranyl, oxazolyl, isoxazolyl, thiazolyl, pyridinyl, pyrimidinyl, pyridazinyl, pyrazinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, benzotriazolyl, 2,3-dihydrobenzo[1,4]dioxinyl, and benzo[1,3]dioxolyl groups. Representative substituted heterocyclyl groups may be mono-substituted or substituted more than once, such as, but not limited to, pyridinyl or morpholinyl groups, which are 2-, 3-, 4-, 5- or 6-substituted, or disubstituted with various groups as defined above, including, but not limited to, alkyl, oxo, carbonyl, amino, alkoxy, cyano, and/or halo.

Other terms may refer to specific groups encompassed by the above definitions. The following terms, while not intended to be limiting, may be used to describe certain combinations of groups. Alkanoyl refers to straight or branched chain alkylcarbonyl groups. Aroyl refers to arylcarbonyl groups. Haloalkyl refers to an alkyl having halogen substituents where halogens are selected from fluorine, chlorine, bromine, or iodine. Haloalkanoyl refers to an alkanoyl group substituted with one or more halogens. Thiol refers to sulfur substituted with hydrogen (—SH). Amino refers to a nitrogen with two hydrogen atoms. Mono-substituted amino refers to a nitrogen with one hydrogen atom and one group selected from alkyl, aryl, or heterocyclyl groups. Di-substituted amino refers to a nitrogen with two groups independently selected from alkyl, aryl, or heterocyclyl groups. Hydroxyalkyl refers to an alkyl group substituted with one or more hydroxyl (—OH) groups. Hydroxyalkenyl refers to an alkenyl group substituted with one or more hydroxyl groups. Thioalkyl refers to an alkyl substituted with one or more thiol groups. Alkoxyalkenyl refers to an alkenyl group substituted with one or more alkyl ether groups. Alkoxyalkyl refers to an alkyl having at least one ether group, alkoxyalkoxyalkyl refers to an alkoxyalkyl group substituted with an alkoxy group, and thus having two or more ether groups, and oxaalkyl generally refers to groups such as alkoxyalkyl, alkoxyalkoxyalkyl, alkoxyalkoxyalkoxyalkyl, and the like. Hydroxyalkylalkoxyalkyl refers to an alkoxyalkyl group substituted with at least one hydroxyalkyl group. Heterocyclylalkyl refers to an alkyl group where one or more hydrogen atoms are replaced by a substituted or unsubstituted heterocyclyl group. Cycloalkylalkyl refers to an alkyl group substituted with a cycloalkyl group. Other combinations of individual groups will be readily apparent to one of skill in the art.

Also included are tautomers. Non-limiting examples of tautomers are keto/enol tautomers, imino/amino tautomers, N-substituted imino/N-substituted amino tautomers, thiol/thiocarbonyl tautomers, and ring-chain tautomers such as the five and six membered ring oxygen, nitrogen, sulfur, or oxygen- and sulfur-containing heterocycles also containing substituents alpha to the heteroatoms. Also specifically included are enantiomers and diastereomers, as well as racemates and isomeric mixtures of the compounds discussed herein.

In one aspect, methods are provided for contacting a mammalian cell (e.g. a human cell) infected with a virus with a first compound, a second compound, and a third compound, wherein the first compound, the second compound, and the third compound are contacted in an amount effective to inhibit the virus. In some embodiments, the first compound may be an iminosugar, such as a compound of Formula I or Formula II, a pharmaceutically acceptable salt thereof, or a mixture of any two or more thereof:

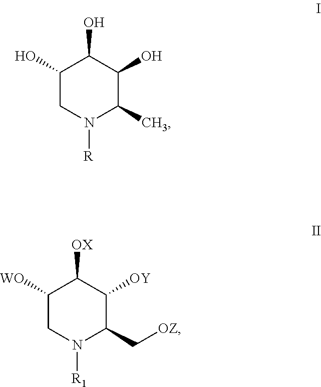

wherein R is selected from substituted or unsubstituted alkyl groups, substituted or unsubstituted cycloalkyl groups, substituted or unsubstituted heterocyclyl groups, or substituted or unsubstituted oxaalkyl groups and where R₁ is selected from substituted or unsubstituted alkyl groups, substituted or unsubstituted cycloalkyl groups, substituted or unsubstituted aryl groups, or substituted or unsubstituted oxaalkyl groups, selected from but not limited to arylalkyl, cycloalkylalkyl, branched or straight chain alkyl groups, and oxaalkyl groups; and where W, X, Y, and Z are each independently selected from hydrogen, alkanoyl groups, aroyl groups, and haloalkanoyl groups. In some such embodiments, the second compound is selected from a nucleotide antiviral compound, a nucleoside antiviral compound, or a mixture of any two or more thereof. In yet other such embodiments, the third compound is selected from an immunostimulating compound, an immunomodulating compound, or a mixture of any two or more thereof.

In some embodiments, when the first compound may be a compound of Formula I, substituted or unsubstituted alkyl groups and/or substituted or unsubstituted oxaalkyl groups may comprise from 1 to 16 carbon atoms, or from 4 to 12 carbon atoms or from 8 to 10 carbon atoms. In some embodiments, substituted or unsubstituted alkyl groups and/or substituted or unsubstituted oxaalkyl groups comprise from 1 to 4 oxygen atoms, and from 1 to 2 oxygen atoms in other embodiments. In other embodiments, substituted or unsubstituted alkyl groups and/or substituted or unsubstituted oxaalkyl groups comprise from 1 to 16 carbon atoms and from 1 to 4 oxygen atoms. Thus, in some embodiments, R is selected from, but is not limited to —(CH₂)₆OCH₃, —(CH₂)₆OCH₂CH₃, —(CH₂)₆O(CH₂)₂CH₃, —(CH₂)₆O(CH₂)₃CH₃, —(CH₂)₂O(CH₂)₅CH₃, —(CH₂)₂O(CH₂)₆CH₃, and —(CH₂)₂O(CH₂)₇CH₃. Other suitable iminosugars and other suitable alkyl and oxaalkyl groups, include those described in PCT application publication No. WO 01/10429.

In some embodiments, the first compound may be a N-substituted-1,5-dideoxy-1,5-imino-D-glucitol compound of Formula II, a pharmaceutically acceptable salt thereof, or a mixture of any two or more thereof: where R1 is selected from substituted or unsubstituted alkyl groups, substituted or unsubstituted cycloalkyl groups, substituted or unsubstituted aryl groups, or substituted or unsubstituted oxaalkyl groups, selected from but not limited to arylalkyl, cycloalkylalkyl, branched or straight chain alkyl groups, and oxaalkyl groups; and where W, X, Y, and Z are each independently selected from hydrogen, alkanoyl groups, aroyl groups, and haloalkanoyl groups. In some such embodiments, R1 is selected from ethyl, propyl, isopropyl, butyl, isobutyl, tert-butyl, pentyl, neopentyl, isopentyl, hexyl, —(CH₂)₂O(CH₂)₅CH₃, —(CH₂)₂O(CH₂)₆CH₃, —(CH₂)₆OCH₂CH₃, and —(CH₂)₂OCH₂CH₂CH₃. In other such embodiments, R1 is butyl, and W, X, Y, and Z are all hydrogen.

In some embodiments, the compound of Formula II is selected from, but is not limited to N-(n-hexyl-)-1,5-dideoxy-1,5-imino-D-glucitol; N-(n-heptyl-)-1,5-dideoxy-1,5-imino-D-glucitol; N-(n-octyl-)-1,5-dideoxy-1,5-imino-D-glucitol; N-(n-octyl-)-1,5-dideoxy-1,5-imino-D-glucitol, tetrabutyrate; N-(n-nonyl-)-1,5-dideoxy-1,5-imino-D-glucitol, tetrabutyrate; N-(n-decyl-)-1,5-dideoxy-1,5-imino-D-glucitol, tetrabutyrate; N-(n-undecyl-)-1,5-dideoxy-1,5-imino-D-glucitol, tetrabutyrate; N-(n-nonyl-)-1,5-dideoxy-1,5-imino-D-glucitol; N-(n-decyl-)-1,5-dideoxy-1,5-imino-D-glucitol; N-(n-undecyl-)-1,5-dideoxy-1,5-imino-D-glucitol; N-(n-dodecyl-)-1,5-dideoxy-1,5-imino-D-glucitol; N-(2-ethylhexyl)-1,5-dideoxy-1,5-imino-D-glucitol; N-(4-ethylhexyl)-1,5-dideoxy-1,5-imino-D-glucitol; N-(5-methylhexyl)-1,5-dideoxy-1,5-imino-D-glucitol; N-(3-propylhexyl)-1,5-dideoxy-1,5-imino-D-glucitol; N-(1-pentylpentylhexyl)-1,5-dideoxy-1,5-imino-D-glucitol; N-(1-butylbutyl)-1,5-dideoxy-1,5-imino-D-glucitol; N-(7-methyloctyl-)-1,5-dideoxy-1,5-imino-D-glucitol; N-(8-methylnonyl)-1,5-dideoxy-1,5-imino-D-glucitol; N-(9-methyldecyl)-1,5-dideoxy-1,5-imino-D-glucitol; N-(10-methylundecyl)-1,5-dideoxy-1,5-imino-D-glucitol; N-(6-cyclohexylhexyl-)-1,5-dideoxy-1,5-imino-D-glucitol; N-(4-cyclohexylbutyl)-1,5-dideoxy-1,5-imino-D-glucitol; N-(2-cyclohexylethyl)-1,5-dideoxy-1,5-imino-D-glucitol; N-(1-cyclohexylmethyl)-1,5-dideoxy-1,5-imino-D-glucitol; N-(1-phenylmethyl)-1,5-dideoxy-1,5-imino-D-glucitol; N-(3-phenylpropyl)-1,5-dideoxy-1,5-imino-D-glucitol; N-(3-(4-methyl)-phenylpropyl)-1,5-dideoxy-1,5-imino-D-glucitol; N-(6-phenylhexyl)-1,5-dideoxy-1,5-imino-D-glucitol; N-(n-nonyl-)-1,5-dideoxy-1,5-imino-D-glucitol, tetrabutyrate; N-(n-decyl-)-1,5-dideoxy-1,5-imino-D-glucitol, tetrabutyrate; N-(n-undecyl-)-1,5-dideoxy-1,5-imino-D-glucitol, tetrabutyrate; N-(n-dodecyl-)-1,5-dideoxy-1,5-imino-D-glucitol, tetrabutyrate; N-(2-ethylhexyl)-1,5-dideoxy-1,5-imino-D-glucitol, tetrabutyrate; N-(4-ethylhexyl)-1,5-dideoxy-1,5-imino-D-glucitol, tetrabutyrate; N-(5-methylhexyl)-1,5-dideoxy-1,5-imino-D-glucitol, tetrabutyrate; N-(3-propylhexyl)-1,5-dideoxy-1,5-imino-D-glucitol, tetrabutyrate; N-(1-pentylpentylhexyl)-1,5-dideoxy-1,5-imino-D-glucitol, tetrabutyrate; N-(1-butylbutyl)-1,5-dideoxy-1,5-imino-D-glucitol, tetrabutyrate; N-(7-methyloctyl-)-1,5-dideoxy-1,5-imino-D-glucitol, tetrabutyrate; N-(8-methylnonyl)-1,5-dideoxy-1,5-imino-D-glucitol, tetrabutyrate; N-(9-methyldecyl)-1,5-dideoxy-1,5-imino-D-glucitol, tetrabutyrate; N-(10-methylundecyl)-1,5-dideoxy-1,5-imino-D-glucitol, tetrabutyrate; N-(6-cyclohexylhexyl-)-1,5-dideoxy-1,5-imino-D-glucitol, tetrabutyrate; N-(4-cyclohexylbutyl)-1,5-dideoxy-1,5-imino-D-glucitol, tetrabutyrate; N-(2-cyclohexylethyl)-1,5-dideoxy-1,5-imino-D-glucitol, tetrabutyrate; N-(1-cyclohexylmethyl)-1,5-dideoxy-1,5-imino-D-glucitol, tetrabutyrate; N-(1-phenylmethyl)-1,5-dideoxy-1,5-imino-D-glucitol, tetrabutyrate; N-(3-phenylpropyl)-1,5-dideoxy-1,5-imino-D-glucitol, tetrabutyrate; N-(3-(4-methyl)-phenylpropyl)-1,5-dideoxy-1,5-imino-D-glucitol, tetrabutyrate; N-(6-phenylhexyl)-1,5-dideoxy-1,5-imino-D-glucitol, tetrabutyrate; pharmaceutically acceptable salts thereof; and mixtures of any two or more thereof.

In some embodiments, the second compound may be selected from, but is not limited to purine nucleotide antiviral compounds, pyrimidine nucleotide antiviral compounds, and mixtures of any two or more thereof. In some embodiments, the second compound is selected from, but is not limited to purine nucleoside antiviral compounds, pyrimidine nucleoside antiviral compounds, and mixtures of any two or more thereof.

Nucleoside and nucleotide compounds, may be based upon purine (III) or pyrimidine (IV) compounds, or analogs thereof, such as compounds V, VI, or VII, with position numbering as shown in Formulas III and IV.

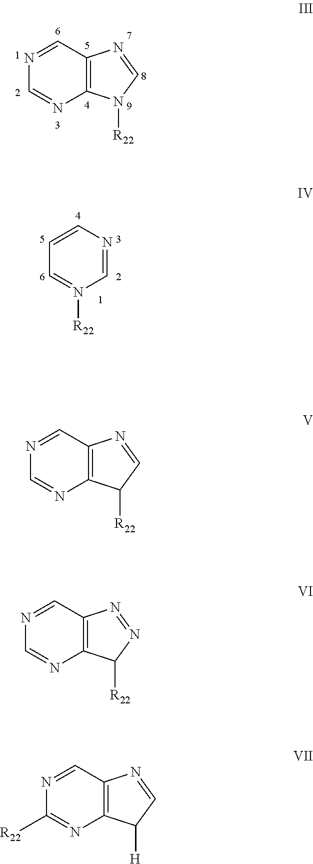

In Formulas III-VII, R₂₂ may be selected from substituted or unsubstituted alkyl groups, substituted or unsubstituted cycloalkyl groups, or substituted or unsubstituted heterocyclyl groups, including but not limited to hydroxyalkyl, hydroxyalkenyl, carboxyalkyl, carboxyalkenyl, thiolalkyl, alkylthioalkyl, alkoxyalkenyl, heterocyclyl, heterocyclylalkyl, hydroxyalkoxyalkyl, oxaalkyl, and cycloalkylalkyl groups. The purine compounds may be further substituted at positions 1, 2, 3, 6, 7, or 8 of the purine heterocycle, and the pyrimidine compounds may be substituted at positions 2, 3, 4, 5, or 6 of the pyrimidine heterocycle. Such substituents may be selected from, but are not limited to hydroxy, alkoxy, halo, thiol, amino, carboxyl, mono-substituted amino, di-substituted amino, and alkyl.

General synthetic methods for the preparation of nucleosides and nucleotides are disclosed in Acta Biochim. Pol., 43, 25-36 (1996); Swed. Nucleosides Nucleotides 15, 361-378 (1996); Synthesis 12, 1465-1479 (1995); Carbohyd. Chem. 27, 242-276 (1995); Chem. Nucleosides Nucleotides 3, 421-535 (1994); Ann. Reports in Med. Chem., Academic Press; and Exp. Opin. Invest. Drugs 4, 95-115 (1995). The chemical reactions described in these references are generally disclosed in terms of their broadest application to the preparation of the compounds. Occasionally, the reactions may not be applicable as described to each compound included within the scope of compounds disclosed herein. The compounds for which this occurs will be readily recognized by those skilled in the art. In all such cases, either the reactions can be successfully performed by conventional modifications known to those skilled in the art, e.g., by appropriate protection of interfering groups, by changing to alternative conventional reagents, by routine modification of reaction conditions, and the like, or other reactions disclosed herein or otherwise conventional will be applicable to the preparation of the corresponding compounds of this invention. In all preparative methods, all starting materials are known or may be prepared from known starting materials.

While nucleoside analogs are generally employed as antiviral agents as is, nucleotides (nucleoside phosphates) may be converted to nucleosides, as is known in the art, in order to facilitate their transport across cell membranes. An example of a chemically modified nucleotide capable of entering cells is S-1-3-hydroxy-2-phosphonylmethoxypropyl cytosine (HPMPC, Gilead Sciences).

Nucleoside and nucleotide compounds are acids and therefore they may also form salts. Examples include salts with alkali metals or alkaline earth metals, such as sodium, potassium, calcium, or magnesium, or with organic bases or basic quaternary ammonium salts. All such salts are intended to be within the scope of the present invention.

Nucleoside and nucleotide compounds thus described, exemplary second compounds include, but are not limited to (+)-cis-5-fluoro-1-[2-(hydroxy-methyl)-[1,3-oxathiolan-5-yl]cytosine; (−)-cis-5-fluoro-1-[2-(hydroxy-methyl)-[1,3-oxathiolan-5-yl]cytosine (FTC); (−)-2′-deoxy-3′-thiocytidine-5′-triphosphate (3TC™, lamivudine); (−)2′,3′, dideoxy-3′-thiacytidine [(−)-SddC]; 1-(2′-deoxy-2′-fluoro-beta-D-arabinofuranosyl)-5-iodocytosine (FIAC); beta-D-arabinofuranosyl)-5-iodocytosine triphosphate 1-(2′-deoxy-2′-fluoro-beta-D-arabinofuranosyl)-5-methyluracil (FIACTP); 1-(2′-deoxy-2′-fluoro-(FMAU); 1-beta-D-ribofuranosyl-1,2,4-triazole-3-carboxamide (ribavirin); 3′-fluoro-5-methyl-deoxycytidine (FddMeCyt); 2′,3′-dideoxy-3′-amino-5-methyl-cytidine; 2′,3′-dideoxy-3′-fluorothymidine beta-L-5-thiacytidine; beta-L-5-fluorocytidine (beta-L-FddC); 2′,3′-dideoxy-(FddThd); 2′,3′-dideoxy-2′,3′-dideoxy-beta-L-5-cytidine (beta-L-ddC); 9-(1,3-dihydroxy-2-propoxymethyl)guanine; 2′-deoxy-3′-thia-5-fluorocytosine; 3′-amino-5-methyl-deoxycytidine (AddMeCyt); 2-amino-1,9-[(2-hydroxymethyl-1-(hydroxymethyl)ethoxy]methyl]-6H-purin-6-one (gancyclovir); 2-[2-(2-amino-9H-purin-9-yl)ethyl]-1,3-propandil diacetate (famciclovir); 2-amino-1,9-dihydro-9-[(2-hydroxy-ethoxy)methyl]-6H-purin-6-one (acyclovir); 9-(4-hydroxy-3-hydroxymethyl-but-1-yl)guanine (penciclovir); 3′-azido-3′-deoxythymidine (AZT™, zidovudine); 3′-chloro-5-methyl-deoxycytidine (ClddMeCyt); 9-(2-phosphonyl-methoxyethyl)-2′,6′-diaminopurine-2′,3′-dideoxyriboside; 9-(2-phosphonylmethoxyethyl)adenine (PMEA); acyclovir triphosphate (ACVTP); D-carbocyclic-2′-deoxyguanosine (CdG); dideoxy-cytidine; dideoxy-cytosine (ddC); dideoxy-guanine (ddG); dideoxy-inosine (ddI); E-5-(2-bromovinyl)-2′-deoxyuridine triphosphate; fluoro-arabinofuranosyl-iodouracil; 1-(2′-deoxy-2′-fluoro-1-beta-D-arabinofuranosyl)-5-iodo-uracil (FIAU); stavudine; 9-beta-D-arabinofuranosyl-9H-purine-6-amine monohydrate (Ara-A); 9-beta-D-arabinofuranosyl-9H-purine-6-amine-5′-monophosphate monohydrate (Ara-AMP); 2-deoxy-3′-thia-5-fluorocytidine; 2′,3′-dideoxy-guanine; 2′,3′-dideoxy-guanosine; or a mixture of any two or more thereof.

A preferred compound may be 1-beta-D-ribofuranosyl-1,2,4-triazole-3-carboxamide (ribavirin).

In some embodiments, the third compound may be selected from an immunostimulating compound, an immunomodulating compound, or a mixture of any two or more thereof. In some such embodiments, the third compound is an interferon. Suitable interferons may be selected from the family of alpha/beta interferons, pegylated interferons such as pegylated interferon alpha-2b (Peg-Intron®) and pegylated interferon alpha 2a (Pegasys®), or mixtures of any two or more interferons. Compounds suitable for use as the third compound may be selected from, but are not limited to AA-2G; adamantylamide; dipeptide; adenosine deaminase, Enzon; adjuvant, Alliance; adjuvants, Ribi; adjuvants, Vaxcel; Adjuvax; agelasphin-11; AIDS therapy, Chiron; algal glucan, SRI; algammulin, Anutech; Anginlyc; anticellular factors, Yeda; Anticort; antigastrin-17 immunogen, Ap; antigen delivery system, Vac; antigen formulation, IDBC; antiGnRH immunogen, Aphton; Antiherpin; Arbidol; Aviron; azarole; Bay-q-8939; Bay-r-1005; BCH-1393; Betafectin; Biostim; BL-001; BL-009; Broncostat; Cantastim; CDRI-84-246; cefodizime; chemokine inhibitors, ICOS; CMV peptides, City of Hope; CN-5888; cytokine-releasing agent, St; DHEAS, Paradigm; DISC TA-KSV; J07B; I01A; I01Z; ditiocarb sodium; ECA-10-142; ELS-1; endotoxin, Novartis; FCE-20696; FCE-24089; FCE-24578; FLT-3 ligand, Immunex; FR-900483; FR-900494; FR-901235; FTS-Zn; G-proteins, Cadus; gludapcin; glutaurine; glycophosphopeptical; GM-2; GM-53; GMDP; growth factor vaccine, EntreM; H-BIG, NABI; H-CIG, NABI; HAB-439; Helicobacter pylori vaccine; herpes-specific immune factor; HIV therapy, United Biomed; HyperGAM+CF; ImmuMax; Immun; BCG; immune therapy, Connective; immunomodulator, Evans; immunomodulators, Novacell; imreg-1; imreg-2; Indomune; inosine pranobex; interferon alpha2, Dong-A; interferon gamma, Genentech; interferon alpha, Novartis; interleukin-12, Genetics; Ins; interleukin-15, Immunex; interleukin-16, Research Cor; ISCAR-1; J005X; L-644257; licomarasminic acid; LipoTher; LK-409; LK-410; LP-2307; LT (R1926); LW-50020; MAF, Shionogi; MDP derivatives, Merck; met-enkephalin, TNI; methylfurylbutyrolactones; MIMP; mirimostim; mixed bacterial vaccine, Tem; MM-1; moniliastat; MPLA, Ribi; MS-705; murabutide; murabutide, Vacsyn; muramyl dipeptide derivative; muramyl peptide derivatives; myelopid; N-563; NACOS-6; NH-765; NISV, Proteus; NPT-16416; NT-002; PA-485; PEFA-814; peptides, Scios; peptidoglycan, Pliva; Perthon, Advanced Plant; PGM derivative, Pliva; Pharmaprojects No. 1099; Pharmaprojects No. 1426; Pharmaprojects No. 1549; Pharmaprojects No. 1585; Pharmaprojects No. 1607; Pharmaprojects No. 1710; Pharmaprojects No. 1779; Pharmaprojects No. 2002; Pharmaprojects No. 2060; Pharmaprojects No. 2795; Pharmaprojects No. 3088; Pharmaprojects No. 3111; Pharmaprojects No. 3345; Pharmaprojects No. 3467; Pharmaprojects No. 3668; Pharmaprojects No. 3998; Pharmaprojects No. 3999; Pharmaprojects No. 4089; Pharmaprojects No. 4188; Pharmaprojects No. 4451; Pharmaprojects No. 4500; Pharmaprojects No. 4689; Pharmaprojects No. 4833; Pharmaprojects No. 494; Pharmaprojects No. 5217; Pharmaprojects No. 530; pidotimod; pimelautide; pinafide; PMD-589; podophyllotoxin, Conpharm; POL-509; poly-ICLC; poly-ICLC, Yamasa Shoyu; PolyA-PolyU; Polysaccharide A; protein A, Berlox Bioscience; PS34WO; pseudomonas MAbs, Teijin; Psomaglobin; PTL-78419; Pyrexol; pyriferone; Retrogen; Retropep; RG-003; Rhinostat; rifamaxil; RM-06; Rollin; romurtide; RU-40555; RU-41821; rubella antibodies, ResCo; S-27609; SB-73; SDZ-280-636; SDZ-MRL-953; SK&F-107647; SL04; SL05; SM-4333; Solutein; SRI-62-834; SRL-172; ST-570; ST-789; staphage lysate; Stimulon; suppressin; T-150R1; T-LCEF; tabilautide; temurtide; Theradigm-HBV; Theradigm-HPV; Theradigm-HSV; THF, Pharm; &; Upjohn; THF, Yeda; thymalfasin; thymic hormone fractions; thymocartin; thymolymphotropin; thymopentin; thymopentin analogues; thymopentin, Peptech; thymosin fraction 5, Alpha; thymostimulin; thymotrinan; TMD-232; TO-115; transfer factor, Viragen; tuftsin, Selavo; ubenimex; Ulsastat; ANGG−; CD-4+; Collag+; COLSF+; COM+; DA-A+; GAST−; GF-TH+; GP-120−; IF+; IF-A+; IF-A-2+; IF-B+; IF-G+; IF-G-1B+; JL-2+; IL-12+; IL-15+; IM+; LHRH−; LIPCOR+; LYM-B+; LYM-NK+; LYM-T+; OPI+; PEP+; PHG-MA+; RNA-SYN−; SY-CW−; TH-A-1+; TH-5+; TNF+; UN; or a mixture of any two or more thereof. A number of sources are available to furnish one or more of the suitable third compounds.

In some embodiments of the methods provided, the contacting a mammalian cell step of the method may comprise administering the first compound, the second compound, and the third compound to a mammal. In other embodiments, methods provide that the first compound, the second compound, and the third compound are administered to the mammal separately, sequentially, or simultaneously. In some such embodiments, the contacting step comprises administering the first compound, the second compound, and the third compound to a human. In addition to the mammalian cell being that of a human, the mammalian cell may be that of a mouse, rat, cat, dog, primate, woodchuck, horse, cow, sheep, pig, camelid, or other mammal in need of treatment of a virus such as a flavivirus, hepadnavirus, or pestivirus. Veterinary uses are envisaged for treatment of such viruses in animals.

In some embodied methods, the virus belongs to the Flaviviridae family of viruses. The virus may be selected from, but is not limited to a hepatitis virus such as hepatitis B virus or hepatitis C virus, or a bovine viral diarrhea virus. In such embodiments, the amount effective to inhibit the virus, is an amount effective to inhibit a hepatitis virus, a hepatitis B virus, a hepatitis C virus, or a bovine viral diarrhea virus.

In another aspect, methods are provided for contacting a mammalian cell with a first compound and a second compound, wherein the first compound and the second compound are contacted in an amount effective to inhibit a virus, and in such embodiments, the second compound is as described above. In other embodiments, the method may further comprise contacting a mammalian cell with a third compound, where the third compound is as described above. In other embodiments, the mammalian cell is a human cell. In yet other embodiments, the virus may be a hepatitis virus which includes, but is not limited to hepatitis B virus and/or hepatitis C virus.

In treating virus infections, one can use the virus combinations or individual compounds disclosed herein in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, undecanoate, and mixtures of any two or more thereof.

In another aspect, kits are provided comprising a first compound, wherein the first compound is a compound of Formula I, Formula II, a pharmaceutically acceptable salt thereof, or a mixture of any two or more thereof, a second compound as described above, and a third compound as described, where the first compound, the second compound, and third compound of the kit are present in an amount effective to inhibit a virus infecting a mammal. In some such embodiments, the first compound, the second compound, and the third compound of the kit form a pharmaceutical composition for simultaneous administration to the mammal. In other such embodiments, the first compound, the second compound, and the third compound of the kit are for separate or sequential administration to the mammal. In yet other embodiments, the second compound and the third compound of the kit comprise a single composition. In some such other embodiments, the first compound and the second compound of the kit comprise a single composition.

In another aspect, compositions are provided comprising a first compound, wherein the first compound is a compound of Formula I, Formula II, a pharmaceutically acceptable salt thereof, or a mixture of any two or more thereof, a second compound, wherein the second compound is as described above, and a third compound as described above, where the first compound, the second compound, and the third compound are in an amount effective to inhibit a virus. In some embodiments, the composition further comprises a pharmaceutically acceptable carrier. In such embodiments, compositions are administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral, as used herein, includes subcutaneous, intravenous, intramuscular, or intrasternal injection, or infusion techniques.

In some embodiments, injectable preparations of the compositions are provided. For example, injectable aqueous or oleaginous suspensions, may be formulated according to the known art using suitable dispersing or wetting agents and suspending agents. In some such embodiments, the injectable preparation is a sterile injectable solution or suspension in a pharmaceutically acceptable diluent, solvent, vehicle, or medium, such as, but not limited to alcohols such as 1,3-butanediol, water, Ringer's solution, isotonic sodium chloride solution, fixed oils such as mono- or diglycerides, fatty acids such as oleic acid, dimethyl acetamide, surfactants including ionic and non-ionic detergents, and polyethylene glycols, or a mixture of any two or more thereof.

Suppositories for rectal administration of the compounds discussed herein may be prepared by mixing the active agent, or agents, with a suitable excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

In some embodiments, dosage forms for oral administration may include capsules, tablets, pills, powders, and granules. In such dosage forms, the compounds may be combined with one or more adjuvants appropriate to the indicated route of administration. In some such embodiments, the compound, or compounds, may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, polyvinyl alcohol, or a mixture of any two or more thereof. In some embodiments, the dosage form may include a controlled-release formulation which may be provided, for example, in a dispersion of active compound in hydroxypropylmethyl cellulose. In the case of capsules, tablets, and pills, the dosage forms can also comprise buffering agents, such as sodium citrate, or magnesium or calcium carbonate or bicarbonate. Tablets and pills can additionally be prepared with enteric coatings.

In other embodiments, formulations for parenteral administration may be in the form of aqueous or non-aqueous isotonic sterile injection solutions or suspensions. In such embodiments, solutions and suspensions may be prepared from sterile powders or granules having one or more of the carriers or diluents mentioned for use in the formulations for oral administration. The compounds may be dissolved in water, polyethylene glycol (PEG), propylene glycol, ethanol, corn oil, cottonseed oil, peanut oil, sesame oil, benzyl alcohol, sodium chloride, various buffers, or a mixture of any two or more thereof. Other adjuvants and modes of administration are well and widely known in the pharmaceutical art.

In other embodiments, liquid dosage forms for oral administration are provided. Such liquid dosage forms may include, but are not limited to, pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The first compounds of Formula I or pharmaceutically acceptable salts thereof, or mixtures of any two or more thereof may be administered in amounts ranging from about 0.01 mg/kg/day to about 1000 mg/kg/day, or from 0.1 to about 100 mg/kg/day or from about 1 mg/kg/day to about 75 mg/kg/day, or from about 5 mg/kg/day to about 50 mg/kg/day.

The first compounds of Formula II or pharmaceutically acceptable salts thereof, or mixtures of any two or more thereof may be administered in amounts ranging from about 0.01 mg/kg/day to about 2500 mg/kg/day, or from 0.1 mg/kg/day to about 500 mg/kg/day or from about 1 mg/kg/day to about 100 mg/kg/day, or from about 5 mg/kg/day to about 50 mg/kg/day.

The second compound may be administered to a human in an amount ranging of from about 0.01 mg/kg/day to about 1000 mg/kg/day or from about 0.1 mg/kg/day to about 200 mg/kg/day or from about 1 mg/kg/day to about 100 mg/kg/day or, from about 2 mg/kg/day to about 50 mg/kg/day, or from about 5 mg/kg/day to about 25 mg/kg/day.

Immunomodulators and immunostimulators may be administered in amounts lower than those conventional in the art. For example, thymosin alpha 1 and thymosin fraction 5 are typically administered to for the treatment of hepatitis B infections in an amount from about 900 μg/m², two times per week (Hepatology (1988) 8:1270; Hepatology (1989) 10:575; Hepatology (1991) 14:409; Gastroenterology (1995) 108:A1127). In some embodiments, doses of thymosin alpha 1 and thymosin fraction 5, two times per week, in amounts ranging from about 10 μg/m2 to about 750 μg/m2, or from about 100 μg/m2 to about 600 μg/m2, in other embodiments, or from about 200 μg/m2 to about 400 μg/m2, in yet other embodiments. Interferon alpha is typically administered for the treatment of hepatitis C infections in an amount from about 1×10⁶ units/person to about 10×10⁶ units/person, three times per week (Simon et al., (1997) Hepatology 25:445-448). Thus, in some embodiments, the dose of interferon alpha is administered three times per week, in the range of from about 0.1×10⁶ units/person to about 7.5×10⁶ units/person, or from about 0.5×10⁶ units/person to about 5×10⁶ units/person, in other embodiments, or from about 1×10⁶ units/person to about 3×10⁶ units/person, in yet other embodiments.

Due to the enhanced hepatitis virus antiviral effectiveness of immunomodulators and immunostimulants in the presence of the compounds of Formula I, Formula II, pharmaceutically acceptable salts thereof, or a mixture of any two or more thereof, reduced amounts of other immunomodulators/immunostimulants may be employed in the methods and compositions disclosed herein to provide broader or more effective antiviral effects. Such reduced amounts may be determined by routine monitoring of hepatitis virus in infected patients undergoing therapy. This may be carried out by, for example, monitoring hepatitis viral DNA or RNA in patients' serum by slot-blot, dot-blot, or PCR techniques, or by measurement of hepatitis surface or other antigens, such as the e antigen, in serum. Methods therefore are discussed in Hoolhagle et al., (1997) New Engl. Jour. Med. 336(5):347-356, and F. B. Hollinger in Fields Virology, Third Ed., Vol. 2 (1996), Bernard N. Fields et al., Eds., Chapter 86, “Hepatitis B Virus,” pp. 2738-2807, Lippincott-Raven, Philadelphia, Pa., and the references cited therein.

Patients may be similarly monitored during combination therapy employing compounds of Formula I or Formula II, pharmaceutically acceptable salts thereof, or a mixture of any two or more thereof, and nucleoside and/or nucleotide antiviral agents to determine the lowest effective doses of each.

The doses described above can be administered to a patient in a single dose or in proportionate multiple subdoses. In the latter case, dosage unit compositions can contain such amounts of submultiples thereof to make up the daily dose. Multiple doses per day can also increase the total daily dose should this be desired by the person prescribing the drug.

One skilled in the art will readily realize that all ranges discussed can and do necessarily also describe all subranges therein for all purposes and that all such subranges also form part and parcel of this invention. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

The present invention also provides a method of treating and/or preventing a viral infection, which includes two subsequent administering steps. The first step involves administering to a subject, such as a mammal and preferably a human, a pharmaceutical combination or composition, that does not inhibit a host enzyme or an ion channel activity. The second step involves administering to the subject the composition and the combination together with a compound, that is at least one of a host enzyme inhibitor or an ion channel activity inhibitor. The first administering step is performed for an amount of time sufficient to enhance the activity of the second administration step. For example, the first administering step can be used to decrease a level of the viral infection significantly, preferably to a non-detectable level. The level of the infection can be determined by taking a sample of a body fluid, such as serum, of the subject and measuring a viral titer in the sample using, for example, RT-PCR or Western blot.

The first step can involve administering at least one of nucleotide or nucleoside antiviral agent and an immunostimulating or immunomodulating agent. Particular compounds administered in the first step can depend on the infection being treated. For example, for Hepatitis C infection, the first step can involve administering interferon and/or ribavirin, while for Hepatitis B or HIV, the first step can involve administering 3TC.

The method can be used for preventing a rebound of the viral infection. For example, after performing the second administering step for a time period sufficient to treat the viral infection, administering the combination or composition used in the first administration step can be withdrawn. After the withdrawal, no rebound of the viral infection occurs in the subject for at least 3 days or for at least 10 days or for at least 30 days.

In some embodiments, the withdrawal of administering the combination or composition used in the first administration step can be accompanied by a withdrawal of administering the compound used in the second administering step in addition to the combination and composition.

Yet in some embodiments, the compound used in the second administering step, i.e. a compound that is at least one of a host enzyme inhibitor or an ion channel inhibitor, can be continued to be administered to the subject after the withdrawal of administering the combination or composition used in the first administration step. In such a case, the compound can be administered in doses lower compared to doses effective for treatment of the viral infection by the compound per se without the first and the second administering steps.

In some embodiments, the compound, that is at least one of an inhibitor of ion channel activity or an host enzyme inhibitor, can be an iminosugar, such as a compound of formula I or formula II, discussed above. The compound can also be castanospermine or a castanospermine derivative, such as celgosivir, also known as, [IS-(1α, 6β, 7α, 8β,8aα)]-octahydro-1,6,7,8-indol-izinetetrol 6-butanoate. Castanospermine and its derivatives are disclosed in U.S. Pat. Nos. and patent publications Nos. 4,970,317; 5,017,563; 5,959,111; 2006/0194835 and PCT publication No. WO0154692.

The host enzyme inhibitor can block a biosynthetic pathway for one or more enzymes in a cell hosting a virus causing the viral infection. The host enzyme inhibitor can be a α-glucosidase inhibitor or α-mannosidase inhibitor. The host enzyme inhibitor may act by interfering with the folding of the viral envelope glycoproteins. Examples of α-glucosidase inhibitors include, but not limited to, N-substituted deoxynojirimycins, such as N-butyl deoxynojirimycin and N-nonyl-deoxynojirimycin, and castanospermine and its derivatives, such as celgosivir. Examples of α-mannosidase inhibitors include, but not limited to, 1,4-dideoxi-1,4-imino-D-mannitol, deoxymannojirimycin, kifunensine, mannostatin A and swainsonine.

Inhibitors of ion channel activity are known to those skilled in the art. For pestiviruses, such as BVDV, and hepaciviruses, such as HCV, an inhibitor of ion channel activity can be a compound inhibiting the activity of p7 protein or an equivalent small membrane spanning protein. Compounds inhibiting ion channel activity and methods of identifying such compounds are disclosed in US patent publication No. 2004/0110795 to Zitzmann and Dwek published Jun. 10, 2004, which is incorporated herein by reference in its entirety.

### Additional Disclosure

The present invention also provides a method of treating a viral infection that includes at least two administration steps/procedures that do not overlap in time. During the first procedure, at least one first antiviral agent is administered to a subject for a first time period, and during the second procedure, the at least one first antiviral agent is administered to the subject together with at least one second antiviral agent for a second time period. The first time period precedes the second time period. The first and the second time periods do not overlap, i.e. the second administering procedure starts after the end of the first time period.

The at least one second antiviral compound can be administered sequentially or concurrently with the at least one first antiviral agent during the second time period.

In some embodiments, the at least one second antiviral agent and the at least one first antiviral agent act on a virus causing or associated with the viral infection via distinct mechanisms. For example, in some embodiments, the at least one first antiviral agent does not inhibit a host enzyme of the virus causing or associated with the viral infection, while the at least one second antiviral agent does inhibit the host enzyme of the virus.

In some embodiments, the at least one first antiviral agent does not inhibit a host alpha-glucosidase of a virus causing or associated with the viral infection, while the at least one second antiviral agent does inhibit the host alpha-glucosidase of the virus.

In some embodiments, the at least one first antiviral agent does not inhibit ion channel activity of a virus causing or associated with the viral infection, while the at least second antiviral agent does inhibit ion channel activity of the virus.

In some embodiments, the at least one first antiviral agent does not include any compound belonging to a certain subclass of compounds, while the at least one second antiviral agent does include a compound belonging to such a subclass. For example, in some embodiments, the at least one first antiviral agent does not include an iminosugar, while the at least one second antiviral agent does include an iminosugar.

In some embodiments, the at least one first antiviral agent does not include a nitrogen-containing compound having formula VIII, while the at least second antiviral agent does include a nitrogen containing compound having formula VIII or a pharmaceutically acceptable salt thereof:

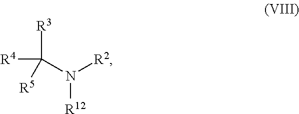

wherein R¹² is an alkyl such as C₁-C₂₀, or C₁-C₆ or C₇-C₁₂ or C₈-C₁₆ and can also contain from 1 to 5 or from 1 to 3 or from 1 to 2 oxygen, R¹² can be an oxa-substituted alkyl derivative.

### First Antiviral Compound

In some embodiments, the at least one first antiviral agent can include one or more compounds selected from immunostimulators and immunomodulators, such as those discussed above; from nucleotide or nucleoside antiviral agents, such as those discussed above; antifibrotic agents, such as an antisense oligonucleotides ISIS-14803™, an anti-tumor necrosis factor α Enbrel® (Etanercept), oral phospholipid antifibrotics IP-501; caspase inhibitors, such as ID-6556 (3-{2-[(2-tert-butyl-phenylaminooxalyl)-amino]-propionylamino}-4-oxo-5-(2,3,5,6-tetrafluoro-phenoxy)-pentanoic acid) and compounds disclosed in U.S. Pat. Nos. 6,004,933, 6,632,962, 6,689,784, 6,800,619 and 7,053,057; inosine 5′-monophosphate dehydrogenase (IMPDH) such as merimepodib (VX-497); inhibitors of viral enzymes, such as viral protease inhibitors and viral polymerase inhibitors; ribozyme and antisense antiviral agents; side effect management agents; and anti-inflammatory agents.

In some embodiments, the at least first antiviral agent can include at least one immunomodulator or immunostimulator. The immunostimulator or immunomodulator can be an immunostimulating or immunomodulating compound described above. Suitable immunomodulators also include thymosin alpha-1 and synthetic versions thereof, such as Zadaxin™ (Thymalfasin); histamine and pharmaceutically acceptable salts thereof, such as histamine dihydrochloride distributed as Ceplene™ by Maxim Pharmaceuticals; viral E1 protein; IC41 vaccine by Intercell; HCV-MF59 vaccine by Chiron.

In some embodiments, the at least one first antiviral agent can include one or more interferon receptor agonists, such as a Type I interferon receptor agonist, a Type II interferon receptor agonist or a Type III interferon receptor agonist.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “a Type II interferon receptor agonist” refers to any naturally-occurring or non-naturally-occurring ligand of a human Type II interferon receptor which binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “a Type III interferon receptor agonist” refers to any naturally-occurring or non-naturally-occurring ligand of a human Type II interferon receptor which binds to and causes signal transduction via the receptor. Type III interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

Type I interferon receptor agonists may include an IFN-α; an IFN-β; an IFN-τ; an IFN-ω; antibody agonists specific for a Type I interferon receptor; and any other agonist of Type I interferon receptor, including non-polypeptide agonists.

Any known IFN-α may be used. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α, (e.g., PEGylated IFN-α; glycosylated IFN-α and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α(including, but not limited to, naturally occurring IFN-α2a; IFN-α2b); recombinant interferon alpha-2b, such as Intron-A interferon available from Schering Corporation, Kenilworth, N.J.; recombinant interferon alpha-2a, such as Roferon interferon available from Hoffmann-La Roche, Nutley, N.J.; recombinant interferon alpha-2C, such as Berofor alpha 2 interferon available from Boehringer Ingelheim Pharmaceutical, Inc., Ridgefield, Conn.; interferon alpha-n1, a purified blend of natural alpha interferons, such as Sumiferon available from Sumitomo, Japan or as Wellferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃, which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods.

Also suitable may be fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See., e.g., Masci et al. (2003) Curr. Oncol. Rep. 5:108-113.

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

In some embodiments, the at least one first antiviral compound may include a known hyperglycosylated polypeptide variant of a parent protein therapeutic. In some embodiments, the parent protein therapeutic is an interferon, and a known hyperglycosylated polypeptide variant comprises (1) a carbohydrate moiety covalently attached to at least one non-native glycosylation site not found in the parent interferon and/or (2) a carbohydrate moiety covalently attached to at least one native glycosylation site found but not glycosylated in the parent interferon.

In some embodiments, the at least one first antiviral agent may include an IFN-β. The term interferon-beta (“IFN-β”) includes IFN-β polypeptides that are naturally occurring; non-naturally-occurring IFN-β polypeptides; and analogs and variants of naturally occurring or non-naturally occurring IFN-β that retain antiviral activity of a parent naturally-occurring or non-naturally occurring IFN-β.

Any of a variety of beta interferons can be used.

Suitable beta interferons include, but are not limited to, naturally-occurring IFN-β; IFN-β1a, e.g., Avonex® (Biogen, Inc.), and Rebif® (Serono, SA); IFN-β1b (Betaseron®; Berlex); and the like. It should be understood that IFN-β may comprise one or modified amino-acid residues such as glycosylations, chemical modifications and the like.

In some embodiments, the at least one first antiviral agent may include an IFN-tau. The term “interferon-tau” (IFN-tau) includes IFN-tau polypeptides that are naturally occurring; non-naturally-occurring IFN-tau polypeptides; and analogs and variants of naturally occurring or non-naturally occurring IFN-tau that retain antiviral activity of a parent naturally-occurring or non-naturally occurring IFN-tau.

Suitable tau interferons include, but are not limited to, naturally-occurring IFN-tau; Tauferon® (Pepgen Corp.); and the like. It should be understood that IFN-tau may comprise one or modified amino-acid residues such as glycosylations, chemical modifications and the like.

In some embodiments, the at least one first antiviral agent can include an IFN-omega. The term interferon-omega (“IFN-ω) includes IFN-ω polypeptides that are naturally occurring; non-naturally-occurring IFN-ω polypeptides; and analogs and variants of naturally occurring or non-naturally occurring IFN-ω that retain antiviral activity of a parent naturally-occurring or non-naturally occurring IFN-ω.

Any known omega interferon may be used. Suitable IFN-ω include, but are not limited to, naturally-occurring IFN-ω; recombinant IFN-ω, e.g., Biomed 510 (BioMedicines); and the like. It should be understood that IFN-ω may comprise one or modified amino-acid residues such as glycosylations, chemical modifications and the like.

In some embodiments, the at least one first antiviral agent may include a Type III interferon receptor agonist. Type III interferon agonists include an IL-28b polypeptide; and IL-28a polypeptide; and IL-29 polypeptide; antibody specific for a Type III interferon receptor; and any other agonist of Type III interferon receptor, including non-polypeptide agonists IL-28A, IL-28B, and IL-29 (referred to herein collectively as “Type III interferons” or “Type III IFNs”) are described in Sheppard et al. (2003) Nature 4:63-68. Each polypeptide can bind a heterodimeric receptor consisting of IL-10 receptor β chain and an IL-28 receptor α. Sheppard et al. (2003), supra. The amino acid sequences of IL-28A, IL-28B, and IL-29 can be found under GenBank Accession Nos. NP-742150, NP-742151, and NP-742152, respectively.

It should be understood that Type III interferon receptor agonist may comprise one or modified amino-acid residues such as glycosylations, chemical modifications and the like. In some embodiments, the at least one first antiviral compound can include a Type II interferon receptor agonist. As used herein, the term “Type II interferon receptor agonist” includes any naturally occurring or non-naturally-occurring ligand of a human Type II interferon receptor that binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

A specific example of a Type II interferon receptor agonist is IFN-gamma and variants thereof. While the present invention exemplifies use of an IFN-gamma polypeptide, it will be readily apparent that any Type II interferon receptor agonist can be used in a subject method. The nucleic acid sequences encoding IFN-gamma polypeptides may be accessed from public databases, e.g., Genbank, journal publications, and the like. While various mammalian IFN-gamma polypeptides are of interest, for the treatment of human, generally the human protein will be used. Human IFN-gamma coding sequence may be found in Genbank, accession numbers X13274; V00543; and NM-000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790. In some embodiments, the IFN-gamma may be glycosylated.

The IFN-gamma may be any of natural IFN-gamma, recombinant IFN-gamma and the derivatives thereof so far as they have an IFN-gamma activity, particularly human IFN-gamma activity.

In some embodiments, the at least one first antiviral agent may include a nucleotide or a nucleoside antiviral agent, such as ribavirin or a derivative thereof. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Ribavirin's manufacture and formulation is described in U.S. Pat. No. 4,211,771. The derivatives of ribavirin include, but not limited to, those described in U.S. Pat. No. 6,277,830. In some embodiments, the at least one first antiviral compound may include levovirin, the L-enantiomer of ribavirin. Levovirin is manufactured by ICN Pharmaceuticals.

In some embodiments, the at least one first antiviral compound may include viramidine, a 3-carboxamidine derivative of ribavirin.

In some embodiments, the at least one first antiviral agent may include a nucleoside or a nucleotide antiviral compound. The term “nucleoside” refers to a compound composed of any pentose or modified pentose moiety attached to a specific position of a heterocycle or to the natural position of a purine (9-position) or pyrimidine (1-position) or to the equivalent position in an analog. The term “nucleotide” refers to a phosphate ester substituted on the 5′-position of a nucleoside. The term “heterocycle” refers to a monovalent saturated or unsaturated carbocyclic radical having at least one hetero atom, such as N, O, S, Se or P, within the ring, each available position of which can be optionally substituted, independently, with, e.g., hydroxyl, oxo, amino, imino, lower alkyl, bromo, chloro and/or cyano. Included within the term “heterocycle” are purines and pyrimidines. The term “purine” refers to nitrogenous bicyclic heterocycles. The term “pyrimidine” refers to nitrogenous monocyclic heterocycles. The term “L-nucleoside” refers to a nucleoside compound that has an L-ribose sugar moiety.

In some embodiments, the nucleoside or nucleotide antiviral compound may be, for example, a nucleoside or nucleotide compound of formula III-VII supra.

In some embodiments, suitable nucleoside compounds include, but not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguanine, 2-amino-α-L-ribofuranl[1′,2′: 4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,2′:4,5]oxazoline, O²,O²-anhydro-β-L-arabinofuranosyluracil, 2′-deoxy-β-L-uridine, 3′5′-Di-O-benzoyl-2′deoxy-4-thioβ-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula I of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both Merck/Isis); and the like.

In some embodiments, the at least one first antiviral agent may include a viral enzyme inhibitor. The viral enzyme inhibitor may be an agent that inhibits an enzymatic activity of an enzyme encoded by the virus. The viral enzyme inhibitor may be a Hepatitis C virus (HCV) enzyme inhibitor. The term “HCV enzyme inhibitor” refers to any agent that inhibits an enzymatic activity of an enzyme encoded by HCV. The term “HCV enzyme inhibitor” includes, but is not limited to, HCV protease inhibitors and HCV polymerase inhibitors. The term “HCV enzyme inhibitor” includes, but is not limited to, agents that inhibit HCV NS3/4A protease activity; agents that inhibit HCV NS3 helicase activity; and agents that inhibit HCV NS5B RNA-dependent RNA polymerase activity.

In some embodiments, the at least one first antiviral agent may include a HCV NS3/4A protease inhibitor. As used herein, the terms “HCV NS3/4A protease inhibitor”, “HCV NS3 protease inhibitor” and “NS3 protease inhibitor” refer to any agent that inhibits the protease activity of HCV NS3/NS4A complex. Unless otherwise specifically stated, the term “NS3 inhibitor” is used interchangeably with the terms “HCV NS3/4A protease inhibitor”, “HCV NS3 protease inhibitor” and “NS3 protease inhibitor.”

Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301 A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54th Ann. Meeting AASLD); NS3 inhibitor SCH6 (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Pat. Nos. 6,732,401, 6,642,204 and 7,091,184; and the like. In some embodiments, the at least one first antiviral agent can include a HCV NS5B inhibitor. As used herein, the terms “HCV NS5B inhibitor,” “NS5B inhibitor,” “HCV NS5B RNA-dependent RNA polymerase inhibitor,” “HCV RDRP inhibitor,” and “RDRP inhibitor,” refer to any agent that inhibits HCV NS5B RNA-dependent RNA polymerase activity. Suitable HCV non-structural protein-5 (NS5; RNA-dependent RNA polymerase) inhibitors include, but are not limited to, a compound as disclosed in U.S. Pat. No. 6,479,508 (Boehringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-2681; a benzothiadiazine compound as disclosed in Dhanak et al. (2002) J. Biol Chem. 277(41):38322-7; an NS5B inhibitor as disclosed in WO 02/100846 A1 or WO 02/100851 A2 (both Shire); an NS5B inhibitor as disclosed in WO 01/85172 A1 or WO 02/098424 A1 (both Glaxo SmithKline); an NS5B inhibitor as disclosed in WO 00/06529 or WO 02/06246 A1 (both Merck); an NS5B inhibitor as disclosed in WO 03/000254 (Japan Tobacco); an NS5B inhibitor as disclosed in EP 1 256,628 A2 (Agouron); JTK-002 (Japan Tobacco); JTK-109 (Japan Tobacco); and the like.

In some embodiments the at least one first antiviral agent may include an inosine 5′-monophosphate dehydrogenase (IMPDH) inhibitor. Suitable IMPDH inhibitors include, but are not limited to, VX-497 ((S)—N-3-[3-(3-methoxy-4-oxazol-5-yl-phenyl)-ureido]-benzyl-carbamic acid tetrahydrofuran-3-yl-ester); Vertex Pharmaceuticals; see, e.g., Markland et al. (2000) Antimicrob. Agents Chemother. 44:859-866); ribavirin; levovirin (Ribapharm; see, e.g., Watson (2002) Curr Opin Investig Drugs 3(5):680-3); viramidine (Ribapharm); and the like.

In some embodiments, the at least one first antiviral compound may include a ribozyme that are complementary to viral nucleotide sequence and/or antisense viral RNA inhibitors. Suitable ribozyme and antisense antiviral agents include, but are not limited to, ISIS 14803 (ISIS Pharmaceuticals/Elan Corporation; see, e.g., Witherell (2001) Curr Opin Investig Drugs. 2(11):1523-9); Heptazyme™; and the like.

In some embodiments, the at least one first antiviral agent and/or the at least one second antiviral agent may include a palliative agent (e.g., an agent that reduces patient discomfort caused by a therapeutic agent), or other agent for the avoidance, treatment, or reduction of a side effect of a therapeutic agent. Such agents are also referred to as “side effect management agents.” Suitable side effect management agents include agents for the avoidance, treatment, or reduction of a side effect of an agent that inhibits enzymatic activity of a membrane-bound α-glucosidase; agents for the avoidance, treatment, or reduction of a side effect of a Type I interferon receptor agonist; agents for the avoidance, treatment, or reduction of a side effect of a Type II interferon receptor agonist; and the like.

Suitable side effect management agents may include agents that are effective in pain management; agents that ameliorate gastrointestinal discomfort; analgesics, anti-inflammatories, antipsychotics, antineurotics, anxiolytics, and hematopoietic agents. In addition, the use of any compound for palliative care of patients suffering from pain or any other side effect in the course of treatment with a subject therapy may be contemplated. Exemplary palliative agents include acetaminophen, ibuprofen, and other non-steroidal anti-inflammatory drugs (NSAIDs), H2 blockers, and antacids.

Analgesics that may be used to alleviate pain may include non-narcotic analgesics, such as NSAIDs acetaminophen, salicylate, acetyl-salicylic acid (aspirin, diflunisal), ibuprofen, Motrin, Naprosyn, Nalfon, and Trilisate, indomethacin, glucametacine, acemetacin, sulindac, naproxen, piroxicam, diclofenac, benoxaprofen, ketoprofen, oxaprozin, etodolac, ketorolac tromethamine, ketorolac, nabumetone, and the like, and mixtures of two or more of the foregoing.

Other suitable analgesics may include fentanyl, buprenorphine, codeine sulfate, morphine hydrochloride, codeine, hydromorphone (Dilaudid®), levorphanol (Levo-Dromoran®), methadone (Dolophine®), morphine, oxycodone (in Percodan®), and oxymorphone (Numorphan®). Also suitable for use are benzodiazepines including, but not limited to, flurazepam (Dalmane®), diazepam (Valium®), and midazolam (Versed®), and the like. Suitable anti-inflammatory agents include, but are not limited to, steroidal anti-inflammatory agents, and non-steroidal anti-inflammatory agents.

Suitable steroidal anti-inflammatory agents include, but are not limited to, hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, conisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures of two or more of the foregoing.

Suitable non-steroidal anti-inflammatory agents, include, but are not limited to, 1) the oxicams, such as piroxicam, isoxicam, tenoxicam, and sudoxicam; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone, mixtures of these non-steroidal anti-inflammatory agents may also be employed, as well as the pharmaceutically-acceptable salts and esters of these agents.

Suitable anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; -Dexamethasone Dipropionate; Diclofenac Potassium; Diclofenac Sodium; Diflorasone Diacetate; -Diflumidone Sodium; Diflunisal; Difluprednate; Diftalone; Dimethyl Sulfoxide; Drocinonide; Endrysone; Enlimomab Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Antipsychotic and antineurotic drugs that may be used to alleviate psychiatric side effects of administering the at least one first antiviral agent and/the at least one second antiviral agent can include any and all selective serotonin receptor inhibitors (SSRIs) and other anti-depressants, anxiolytics (e.g. alprazolam), etc. Anti-depressants include, but are not limited to, serotonin reuptake inhibitors, such as Celexa® (citalopram), Desyrel® (trazodone), Effexor® (venlafaxine), Luvox® (fluvoxamine), Paxil® (paroxetine), Prozac® (fluoxetine), Zoloft® (sertraline), and Serzone® (nefazodone); tricyclics, such as Adapin® (doxepin), Anafrinil® (clomipramine), Elavil® (amitriptyline), Janimine® (Imipramine), Ludiomil® (maprotiline), Pamelor® (nortriptyline), Tofranil® (imipramine), Vivactil® (protriptyline), Sinequan® (doxepin) and Surmontil® (trimipramine); monoamine oxidase inhibitors such as Eldepryl® (selegine), Marplan® (isocarboxazid), Nardil® (phenelzyne) and Parnate® (tranylcypromine). Anti-anxiety agents include, but are not limited to, azaspirones, such as BuSpar® (buspirone); benzodiazepines such as Ativan® (lorazepan), Librium® (chlordiazepoxide), Tranxene® (clorazepate), Centrax® (prazepam), Klonopin® (clonozepam), Paxipam® (halazepam), Serax® (oxazepam), Valium® (diazepam) and Xanax® (alprozolam) and beta-blockers, such as Inderal® (propanolol) and Tenormin® (atenolol).

Agents that reduce gastrointestinal discomfort, such as nausea, diarrhea, gastrointestinal cramping, and the like are suitable palliative agents for use in a subject combination therapy. Suitable agents include, but are not limited to, antiemetics, anti-diarrheal agents, H2 blockers, antacids, and the like.

Suitable H2 blockers (histamine type 2 receptor antagonists) that are suitable for use as a palliative agent in a subject therapy include, but are not limited to, Cimetidine (e.g., Tagamet®, Peptol®, Nu-cimet®, Apo-cimetidine®); Ranitidine (e.g., Zantac®, Nu-ranit®, Novo-randine®, and Apo-ranitidine®); and Famotidine (Pepcid®, Apo-Famotidine®, and Novo-Famotidine®).

Suitable antacids include, but are not limited to, aluminum and magnesium hydroxide (Maalox®, Mylanta®); aluminum carbonate gel (Basajel®); aluminum hydroxide (Amphojel®, AlternaGEL®); calcium carbonate (Tums®, Titralac®); magnesium hydroxide; and sodium bicarbonate.

Antiemetics include, but are not limited to, 5-hydroxytryptophan-3 (5HT3) inhibitors; corticosteroids such as dexamethasone and methylprednisolone; Marinol® (dronabinol); prochlorperazine; benzodiazepines; promethazine; and metoclopramide cisapride; Alosetron Hydrochloride; Batanopride Hydrochloride; Bemesetron; Benzquinamide; Chlorpromazine; Chlorpromazine Hydrochloride; Clebopride; Cyclizine Hydrochloride; Dimenhydrinate; Diphenidol; Diphenidol Hydrochloride; Diphenidol Pamoate; Dolasetron Mesylate; Domperidone; Dronabinol; Fludorex; Flumeridone; Galdansetron Hydrochloride; Granisetron; Granisetron Hydrochloride; Lurosetron Mesylate; Meclizine Hydrochloride; Metoclopramide Hydrochloride; Metopimazine; Ondansetron Hydrochloride; Pancopride; Prochlorperazine; Prochlorperazine Edisylate; Prochlorperazine Maleate; Promethazine Hydrochloride; Thiethylperazine; Thiethylperazine Malate; Thiethylperazine Maleate; Trimethobenzamide Hydrochloride; Zacopride Hydrochloride.

Anti-diarrheal agents include, but are not limited to, Rolgamidine, Diphenoxylate hydrochloride (Lomotil®), Metronidazole (Flagyl®), Methylprednisolone (Medrol®), Sulfasalazine (Azulfidine®), and the like.

Suitable hematopoietic agents that can be used to prevent or restore depressed blood cell populations in the methods of the invention include erythropoietins, such as EPOGEN™ epoetin-alfa, granulocyte colony stimulating factors (G-CSFs), such as NEUPOGEN™ filgrastim, granulocyte-macrophage colony stimulating factors (GM-CSFs), thrombopoietins, etc.

In some embodiments, the at least one first antiviral agent may include more than one first antiviral agents. For example, in some embodiments, the at least one first antiviral agent may include an interferon receptor agonist, such as Type I interferon receptor agonist, and a nucleoside or nucleotide antiviral agent, such as ribavirin. When the at least one first antiviral agent includes more than one first antiviral agents, such antiviral agents may be administered concurrently or sequentially during the first time period and during the second time period.

### Second Antiviral Agent

In some embodiments, the at least one second antiviral agent may comprise an alpha-glucosidase inhibitor. The alpha-glucosidase inhibitor can be an agent that inhibits host alpha-glucosidase enzymatic activity by at least about 10%, at least about 15%, at least about 20%, at least about 25%, at least about 30%, at least about 35%, at least about 40%, at least about 50%, at least about 60%, at least about 70%, at least about 80%, or at least about 90%, or more, compared to the enzymatic activity of the alpha-glucosidase in the absence of the agent. The term “alpha-glucosidase inhibitot” encompasses both naturally occurring and synthetic agents that inhibit host alpha-glucosidase activity.

Suitable alpha-glucosidase inhibitors include, but not limited to, deoxynojirimycin and N-substituted deoxynojirimycins, such as compounds of Formula II and pharmaceutically acceptable salts thereof. In some embodiments, N-alkylated deoxynojirimycins, such as N-butyl deoxynojirimycin and N-nonyl deoxynojirimycin can be preferred.

Suitable alpha-glucosidase inhibitors also include N-oxaalkylated deoxynojirimycins, such as N-hydroxyethyl DNJ (Miglitol or Glyset®) described in U.S. Pat. No. 4,639,436.

Suitable alpha-glucosidase inhibitors also include castanospermines and castanospermine derivatives, such as compounds of Formula (I) and pharmaceutically acceptable salts thereof disclosed in US patent application No. 2006/0194835, including 6-O-butanoyl castanospermine (celgosivir), compounds of Formula II disclosed in PCT publication No. WO01054692 and pharmaceutically acceptable salts thereof.

In some embodiments, the alpha glucosidase inhibitor may be acarbose (O-4,6-dideoxy-4-[[(1S,4R,5S,6S)-4,5,6-trihydroxy-3-(hydroxymethyl)-2-cyc-lohexen-1-yl]amino]-α-D-glucopyranosyl-(1→4)—O—→-D-gluc-opyranosyl-(1→4)-D-glucose), or Precose®. Acarbose is disclosed in U.S. Pat. No. 4,904,769. In some embodiments, the alpha glucosidase inhibitor can be a highly purified form of acarbose (see, e.g., U.S. Pat. No. 4,904,769).

In some embodiments, the at least one second antiviral agent may include at least one ion channel inhibitor. In some embodiments, the ion channel inhibitor may be an agent inhibiting the activity of HCV p7 protein. Ion channel inhibitors and methods of identifying them are detailed in U.S. Pat. No. 7,256,005. For example, the ion channel inhibitor may be a compound of formula I or formula II of U.S. Pat. No. 7,256,005.

In some embodiments, the ion channel inhibitor may be a compound of formula IX or X or a pharmaceutically acceptable salt thereof:

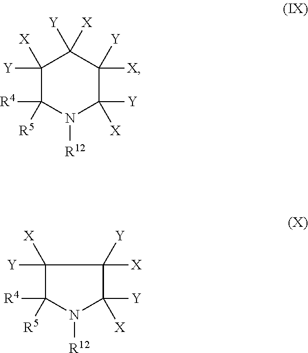

wherein each X and each Y, independently selected from the group consisting of —H; —OH; —F; —Cl; —Br; —I; —NH₂; alkyl- and dialkylamino; linear or branched C₁₋₆ alkyl, C₂₋₆ alkenyl and alkynyl; aralkyl; linear or branched C₁₋₆ alkoxy; aryloxy; aralkoxy; -(alkylene)oxy(alkyl); —CN; —NO₂; —COOH; —COO(alkyl); —COO(aryl); —C(O)NH(C₁₋₆ alkyl); —C(O)NH(aryl); sulfonyl; (C₁₋₆ alkyl)sulfonyl; arylsulfonyl; sulfamoyl, (C₁₋₆ alkyl)sulfamoyl; (C₁₋₆ alkyl)thio; (Cl_{—}6 alkyl)sulfonamide; arylsulfonamide; —NHNH₂; —NHOH; aryl; and heteroaryl, wherein each of the substituents may be the same or different, wherein R⁴ is hydrogen or deleted (i.e. not present); R⁵ is hydrogen, hydroxyl, amino, a substituted amino, carboxy, an alkoxycarbonym, an aminocarbonyl, an alkyl, an aryl, an aralkyl, a hydroxyalkyl, an acyloxy or an aroyloxy and wherein R¹² is an alkyl such as C₅₋₁₈ alkyl, or C₇₋₁₂ alkyl, or C₈₋₁₆ alkyl or an oxa-alkylated alkyl derivative, i.e. an alkyl containing from 1 to 5 or from 1 to 3 or from 1 to 2 oxygen atoms.

In some embodiments, the ion channel inhibitor may be N-alkyl-1,5-dideoxy-1,5-imino-D-galactitol (N-alkyl-DGJ) or N-oxa-alkyl-1,5-dideoxy-1,5-imino-D-galactitol (N-oxa-alkyl-DGJ) having the formula:

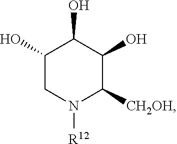

wherein R¹² is an alkyl such as C₅₋₁₈ alkyl, or C₇₋₁₂ alkyl, or C₈₋₁₆ alkyl or an oxa-alkylated alkyl derivative, i.e. an alkyl containing from 1 to 5 or from 1 to 3 or from 1 to 2 oxygen atoms

In some embodiments, the ion channel inhibitor may be N-alkyl-1,5,6-trideoxy-1,5-imino-D-galactitol (N-alkyl-MeDGJ) or N-oxa-alkyl-1,5,6-trideoxy-1,5-imino-D-galactitol having (N-oxa-alkyl-MeDGJ) having the formula:

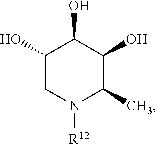

wherein R¹² is an alkyl such as C₅₋₁₈ alkyl, or C₇₋₁₂ alkyl, or C₈₋₁₆ alkyl or an oxa-alkylated alkyl derivative, i.e. an alkyl containing from 1 to 5 or from 1 to 3 or from 1 to 2 oxygen atoms.

In some embodiments, the ion channel inhibitor may be N-alkyl or N-oxa-alkyl substituted deoxynojirimycin having formula:

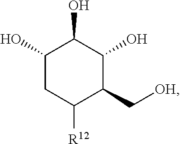

wherein R¹² is an alkyl such as C₅₋₁₈ alkyl, or C₇₋₁₂ alkyl, or C₈₋₁₆ alkyl or an oxa-alkylated alkyl derivative, i.e. an alkyl containing from 1 to 5 or from 1 to 3 or from 1 to 2 oxygen atoms.

Suitable ion channel inhibitors include, but not limited to, N-(7-oxa-nonyl)-1,5,6-trideoxy-1,5-imino-D-galactitol (N-7-oxa-nonyl 6-MeDGJ or UT231B), N-10-oxaundecul-6-MeDGJ, N-nonyl deoxynojirimycin, N-nonyl deoxynogalactonojirimycin and N-oxanonyl deoxynogalactonojirimycin.

In some embodiments, the at least one second antiviral agent may include an iminosugar. Suitable iminosugars include both naturally occurring iminosugars and synthetic iminosugars.

In some embodiments, the iminosugar may be deoxynojirimycin or N-substituted deoxynojirimycin derivative. Examples of suitable N-substituted deoxynojirimycin derivatives include, but not limited to, compounds of Formula II of the present application, compounds of Formula I of U.S. Pat. No. 6,545,021 and N-oxaalkylated deoxynojirimycins, such as N-hydroxyethyl DNJ (Miglitol or Glyset®) described in U.S. Pat. No. 4,639,436.

In some embodiments, the iminosugar may be castanospermine or castanospermine derivative. Suitable castanospemine derivatives include, but not limited to, compounds of Formula (I) and pharmaceutically acceptable salts thereof disclosed in US patent application No. 2006/0194835 and compounds and pharmaceutically acceptable salt thereof of Formula II disclosed in PCT publication No. WO01054692.

In some embodiments, the iminosugar may be deoxynogalactojirimycin or N-substituted derivative thereof, such as those disclosed in PCT publications Nos. WO99/24401 and WO01/10429. Examples of suitable N-substituted deoxynogalactojirimycin derivatives include, but not limited to, N-alkylated deoxynogalactojirimycins (N-alkyl-1,5-dideoxy-1,5-imino-D-galactitols), such as N-nonyl deoxynogalactojirimycin, and N-oxa-alkylated deoxynogalactojirimycins (N-oxa-alkyl-1,5-dideoxy-1,5-imino-D-galactitols), such as N-7-oxanonyl deoxynogalactojirimycin.

In some embodiments, the iminosugar may be N-substituted 1,5,6-trideoxy-1,5-imino-D-galactitol (N-substituted MeDGJ) including, but not limited to compounds of Formula I. N-substituted MeDGJs are disclosed, for example, in PCT publication No. WO01/10429.

In some embodiments, the at least second antiviral agent may include a nitrogen containing compound having formula VIII or a pharmaceutically acceptable salt thereof:

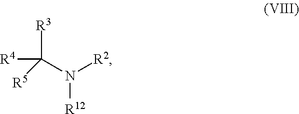

wherein R¹² is an alkyl such as C₁-C₂₀, or C₁-C₆ or C₇-C₁₂ or C₈-C₁₆ and can also contain from 1 to 5 or from 1 to 3 or from 1 to 2 oxygen, R¹² can be an oxa-substituted alkyl derivative. Examples if oxa-substituted alkyl derivatives include 3-oxanonyl, 3-oxadecyl, 7-oxanonyl and 7-oxadecyl.
R² is hydrogen, R³ is carboxy, or a C₁-C₄ alkoxycarbonyl, or R² and R³, together
are

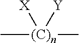

or —(CXY)ₙ—, wherein n is 3 or 4, each X, independently, is hydrogen, hydroxy, amino, carboxy, a C₁-C₄ alkylcarboxy, a C₁-C₄ alkyl, a C₁-C₄ alkoxy, a C₁-C₄ hydroxyalkyl, a C₁-C₆ acyloxy, or an aroyloxy, and each Y, independently, is hydrogen, hydroxy, amino, carboxy, a C₁-C₄ alkylcarboxy, a C₁-C₄ alkyl, a C₁-C₄ alkoxy, a C₁-C₄ hydroxyalkyl, a C₁-C₆ acyloxy, an aroyloxy, or deleted (i.e. not present);
R⁴ is hydrogen or deleted (i.e. not present); and
R⁵ is hydrogen, hydroxy, amino, a substituted amino, carboxy, an alkoxycarbonyl, an aminocarbonyl, an alkyl, an aryl, an aralkyl, an alkoxy, a hydroxyalkyl, an acyloxy, or an aroyloxy, or R³ and R⁵, together, form a phenyl and R⁴ is deleted (i.e. not present).

In some embodiments, the nitrogen containing compound has the formula:

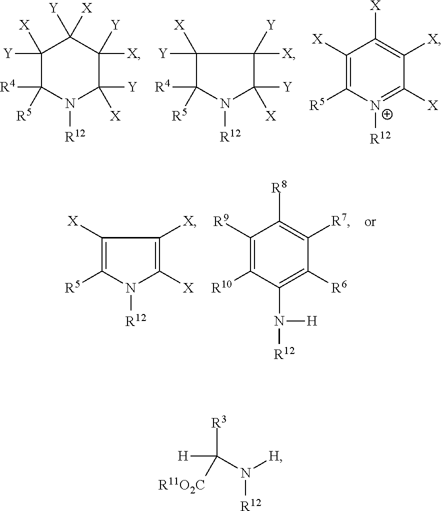

where each of R⁶—R¹⁰, independently, is selected from the group consisting of hydrogen, hydroxy, amino, carboxy, C₁-C₄ alkylcarboxy, C₁-C₄ alkyl, C₁-C₄ alkoxy, C₁-C₄ hydroxyalkyl, C₁-C₄ acyloxy, and aroyloxy; and R¹¹ is hydrogen or C₁-C₆ alkyl.

The nitrogen-containing compound may be N-alkylated piperidine, N-oxa-alkylated piperidine, N-alkylated pyrrolidine, N-oxa-alkylated pyrrolidine, N-alkylated phenylamine, N-oxa-alkylated phenylamine, N-alkylated pyridine, N-oxa-alkylated pyridine, N-alkylated pyrrole, N-oxa-alkylated pyrrole, N-alkylated amino acid, or N-oxa-alkylated amino acid. In certain embodiments, the N-alkylated piperidine, N-oxa-alkylated piperidine, N-alkylated pyrrolidine, or N-oxa-alkylated pyrrolidine compound can be an iminosugar. For example, in some embodiments, the nitrogen-containing compound may be N-alkyl-1,5-dideoxy-1,5-imino-D-galactitol (N-alkyl-DGJ) or N-oxa-alkyl-1,5-dideoxy-1,5-imino-D-galactitol (N-oxa-alkyl-DGJ) having the formula:

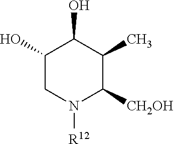

or N-alkyl-1,5,6-trideoxy-1,5-imino-D-galactitol (N-alkyl-MeDGJ) or N-oxa-alkyl-1,5,6-trideoxy-1,5-imino-D-galactitol having (N-oxa-alkyl-MeDGJ) having the formula:

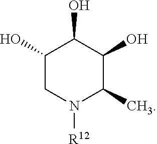

As used herein, the groups have the following characteristics, unless the number of carbon atoms is specified otherwise. Alkyl groups may have from 1 to 20 carbon atoms and may be linear or branched, substituted or unsubstituted. Alkoxy groups may have from 1 to 16 carbon atoms, and may be linear or branched, substituted or unsubstituted. Alkoxycarbonyl groups may be ester groups having from 2 to 16 carbon atoms. Alkenyloxy groups may have from 2 to 16 carbon atoms, from 1 to 6 double bonds, and may be linear or branched, substituted or unsubstituted. Alkynyloxy groups may have from 2 to 16 carbon atoms, from 1 to 3 triple bonds, and may be linear or branched, substituted or unsubstituted. Aryl groups may have from 6 to 14 carbon atoms (e.g., phenyl groups) and may be substituted or unsubstituted. Aralkyloxy (e.g., benzyloxy) and aroyloxy (e.g., benzoyloxy) groups may have from 7 to 15 carbon atoms and are substituted or unsubstituted. Amino groups may be primary, secondary, tertiary, or quaternary amino groups (i.e., substituted amino groups). Aminocarbonyl groups may be amido groups (e.g., substituted amido groups) having from 1 to 32 carbon atoms. Substituted groups may include a substituent selected from the group consisting of halogen, hydroxy, C₁₋₁₀ alkyl, C₂₋₁₀ alkenyl, C₁₋₁₀ acyl, or C₁₋₁₀ alkoxy.

The N-alkylated amino acid may be an N-alkylated naturally occurring amino acid, such as an N-alkylated a-amino acid. A naturally occurring amino acid is one of the 20 common α-amino acids (Gly, Ala, Val, Leu, Ile, Ser, Thr, Asp, Asn, Lys, Glu, Gln, Arg, His, Phe, Cys, Trp, Tyr, Met, and Pro), and other amino acids that are natural products, such as norleucine, ethylglycine, ornithine, methylbutenyl-methylthreonine, and phenylglycine. Examples of amino acid side chains (e.g., R⁵) include H (glycine), methyl (alanine), —CH₂C(O)NH₂ (asparagine), —CH₂—SH (cysteine), and —CH(OH)CH₃ (threonine).

An N-alkylated compound can be prepared by reductive alkylation of an amino (or imino) compound. For example, the amino or imino compound can be exposed to an aldehyde, along with a reducing agent (e.g., sodium cyanoborohydride) to N-alkylate the amine. Similarly, a N-oxa-alkylated compound can be prepared by reductive alkylation of an amino (or imino) compound. For example, the amino or imino compound can be exposed to an oxa-aldehyde, along with a reducing agent (e.g., sodium cyanoborohydride) to N-oxa-alkylate the amine.

The nitrogen-containing compound may include one or more protecting groups. Various protecting groups are well known. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthetic transformations are complete. Clearly, where a compound differs from a compound disclosed herein only in that one or more protecting groups of the disclosed compound has been substituted with a different protecting group, that compound is within the invention. Further examples and conditions are found in Greene, Protective Groups in Organic Chemistry, (1^{st} Ed., 1981, Greene & Wuts, 2^{nd} Ed., 1991).

The nitrogen-containing compound may be purified, for example, by crystallization or chromatographic methods. The compound can be prepared stereospecifically using a stereospecific amino or imino compound as a starting material.

The amino and imino compounds used as starting materials in the preparation of the long chain N-alkylated compounds are commercially available (Sigma, St. Louis, Mo.; Cambridge Research Biochemicals, Norwich, Cheshire, United Kingdom; Toronto Research Chemicals, Ontario, Canada) or can be prepared by known synthetic methods. For example, the compounds may be N-alkylated imino sugar compounds or oxa-substituted derivatives thereof. The imino sugar can be, for example, deoxygalactonojirmycin (DGJ), 1-methyl-deoxygalactonojirimycin (MeDGJ), deoxynorjirimycin (DNJ), altrostatin, 2R,5R-dihydroxymethyl-3R,4R-dihydroxypyrrolidine (DMDP), or derivatives, enantiomers, or stereoisomers thereof.

The syntheses of a variety of iminosugar compounds have been described. For example, methods of synthesizing DNJ derivatives are known and are described, for example, in U.S. Pat. Nos. 5,622,972, 5,401,645, 5,200,523, 5,043,273, 4,994,572, 4,246,345, 4,266,025, 4,405,714, and 4,806,650. Methods of synthesizing other iminosugar derivatives are known and are described, for example, in U.S. Pat. Nos. 4,861,892, 4,894,388, 4,910,310, 4,996,329, 5,011,929, 5,013,842, 5,017,704, 5,580,884, 5,286,877, and 5,100,797 and PCT publication No. WO 01/10429. The enantiospecific synthesis of 2R,5R-dihydroxymethyl-3R,4R-dihydroxypyrrolidine (DMDP) is described by Fleet & Smith (Tetrahedron Lett. 26:1469-1472, 1985).

### Viral Infections

The method of the present invention may be applied for treatment of a variety of viral infections.

In some embodiments, the viral infection may be a viral infection caused by or associated with an alphavirus, i.e. a virus belonging to the family Alphaviridae, which includes influenza viruses, parafluenza viruses, picornaviruses, polio virus, flaviviruses, such as yellow fever virus, the four serotypes of dengue virus, West Nile virus, hepatitis viruses, and many other disease causing viruses. As used herein, the term “alphavirus” and its grammatical variants refer to a group of viruses characterized by (a) an RNA genome, (ii) viral replication in cytoplasm of host cells and (iii) no DNA phase occurs in the viral replication cycle.

In some embodiments, the virus may be a hepatitis virus, such as Hepatitis A virus, Hepatitis B virus, Hepatitis C virus, Hepatitis D virus, Hepatitis E, Hepatitis G virus or a bovine diarrhea virus.

The present inventions may be particularly applicable for treating Hepatitis C viral infection/

### First Time Period

A duration of the first time period may vary depending on a variety of parameters including the particular at least one first antiviral agent administered to the subject and parameters of the viral infection in the subject such as type of the viral infection, genotype and subgenotype of the virus causing or associated with the viral infection and initial pretreatment viral load in the subject.

In some embodiments, a duration of the first time period may range from about 1 to about 60 weeks or from about 2 to about 60 weeks or from about 4 to about 60 weeks or from about 8 weeks to about 60 weeks or from about 12 weeks to about 60 weeks or from about 18 weeks to about 60 weeks or from about 24 weeks to about 60 weeks or from about 24 weeks to about 48 weeks. In some embodiments, the first time period may be about 24 weeks or about 48 weeks.

In some embodiments, a duration of the first time period may be determined by measuring a viral response in the subject to the administering the at least one first antiviral agent. An end of the first time period may be triggered by a time the viral response in the subject reaches a certain predetermined level. Evaluation of the viral response may be performed, for example, by measuring a viral load of the infection in the subject or by measuring a parameter associated with the viral infection. For example, for HCV infection such a parameter may include one or more of the following parameters: liver fibrosis, elevations in serum transminase levels and necroinflamatory activity in the liver.

In some embodiments, a level of the viral load that triggers the end of the first time period may be an undetectable level of the viral load. In some embodiments, the first time period may end and the second time period can start right after, e.g. the next day, the certain predetermined level of the viral load is reached in the subject. Yet in some embodiments, the first time period may end and the second time period may start after the certain predetermined level of the viral load is sustained in the subject for a certain predetermined time period. Such a certain predetermined time period may range, for example, from about 1 week to about 24 weeks or from about 2 weeks to about 12 weeks.

In some embodiments, the first time period may be set or determined in advance based on the particular at least one first viral agent administered and/or parameters of the viral infection in the subject. For example, in some embodiments, when the at least one first antiviral agent comprises pegylated interferon and ribavirin, the first period may be set to be about 24 weeks for patients with genotype II or III of HCV, and 48 weeks for patients with genotype I of HCV.

### Viral Response Evaluation

In some embodiments, the method of the present invention may include evaluation of a viral response to the treatment in the subject. A variety of techniques exist for the evaluation of the viral response. Particular techniques may vary depending on a particular viral infection being treated.

The evaluation of the viral response may be performed at any time. In some embodiments, the evaluation of the viral response at the end of the first time period may be preferred.

In some embodiments, the evaluation may be performed multiple times during the first time period.

In some embodiments, the evaluation may start at a certain time before the end of the first time period. For example, in certain embodiments, when the first time period lasts about 48 weeks, the evaluation may start on week 36 or week 40 and be performed periodically on weekly or biweekly basis.

In some embodiments, the viral response may be evaluated by measuring a viral load, i.e. a titer or level of virus in serum or other body fluid or body tissue of the subject. Methods of measuring the titer or the level of the virus in serum or other body fluid or body tissue include, but are not limited to, a quantitative polymerase chain reaction (PCR) and a branched DNA (bDNA) test.

For evaluating a viral response in treatment of Hepatitis C infection, one may use quantitative assays for measuring the viral load (titer) of HCV RNA. Many such assays are available commercially, including a quantitative reverse transcription PCR(RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Gen-Probe Inc. (San Diego) and Chiron Corporation, and sold by Chiron Corporation under the trade name Procleix®, which NAT simultaneously tests for the presence of HIV-1 and HCV. See, e.g., Vargo et al. (2002) Transfusion 42:876-885.

In some embodiments, for Hepatitis C viral infection, the viral response may be determined by measuring a parameter associated with HCV infection, such as liver fibrosis. Liver fibrosis may be evaluated using non-invasive tests measuring parameters, such as liver-associated chemistries, platelet count, prothrombin time and specific serum markers of fibrosis. Methods of determining degree of liver fibrosis are discussed, for example, in paragraphs 0091-0110 of US patent publication No. 2006/0269517.

In some embodiments, for Hepatitis C viral infection, the viral response may be determined by measuring a level of serum alanine aminotransferase (ALT) using, for example, a standard essay. In general, an ALT level of less than about 45 international units (IU) per milliliter is considered to be normal.

### Relapse Prevention

In some embodiments, the second administration, i.e. administration of both the at least one first antiviral agent and the at least one second antiviral agent may be performed only to those subjects that exhibit a favorable viral response after the first time period. In some embodiments, the favorable viral response means that a level or titer of the viral infection in the subject became negative, in other words a level or titer of the viral infection in the subject was reduced in serum or other body fluid of the subject to an undetectable level. For Hepatitis C viral infection, the undetectable level of the viral load may be an HCV RNA viral load of less than about 5000, less than about 1000, less than about 500, less than about 200 or preferably less than about 100 genome copies/mL serum or other body fluid. In some embodiments, the favorable viral response means that a parameter associated with the viral infection reached a normal level following the treatment. For example, for Hepatitis C viral infection, the favorable viral response can mean that an ALT level in the subject reduced to less than about 45 IU/ml.

In the embodiments, where the second administration may be performed only to those subjects that exhibit a negative viral titer or load after the first time period, the method of the present invention can serve for prevention of relapse of the viral infection in such subjects, i.e. for prevention of reappearance of the viral infection in the subject.

As used herein, the term “relapse rate” refers between a number of subjects, who had a negative viral load at the end of the treatment but did not sustain the negative viral load after a certain period of time, to a total number of subjects who had a negative viral load at the end of the treatment.

Relapse prevention may be of particular importance for Hepatitis C infection treatment. For example, for chronically infected with genotype 1 HCV subjects that received peginterferon-α2a (180 μg/week) and Ribavirin (1000 or 1200 mg/d) treatment for 48 weeks, a relapse rate was about 25% 24 weeks after the treatment's end. For chronically infected with genotype 1 HCV subjects that received peginterferon-α2a (180 μg/week) and Ribavirin (1000 or 1200 mg/d) treatment for 48 weeks and initially a high HCV viral load (>2×10⁶ copies/ml), a relapse rate was about 28% 24 weeks after the treatment's end. For chronically infected with genotype 1 HCV subjects that received peginterferon-α2a (180 μg/week) and a lower dose of Ribavirin (800 mg/d) treatment for 48 weeks, a relapse rate was about 32% 24 weeks after the treatment's end, see Hadziyannis S J, Sette H., Morgan T. R., et al. Peginterferon-α2a and Ribavirin Combination Therapy in Chronic Hepatitis C. Ann Intern Med. 2004; 140:346-355.

The second administration, i.e. administration of the at least one first antiviral compound and the at least one second antiviral compound, following the first administration may reduce a relapse rate compared to treatment that involves only the first administration and does not include the second administration, i.e. does not include an administration of the at least one second antiviral agent and the at least one first antiviral agent during the same time period.

### Second Time Period

A duration of the second time period may vary depending on factors that include parameters of the viral infection in the subject and particular the at least one first antiviral agent and the at least one second viral agent administered to the subject. In some embodiments, the duration of the second time period may range from about 1 week to about 60 weeks or from about 2 weeks to about 48 weeks or from about 2 weeks to about 24 weeks or from about 4 to about 12 weeks.

### Third Time Period

In some embodiments, after the end of the second time period, administering of the at least one first antiviral agent may be withdrawn. In some embodiments, the withdrawal of administering the at least one first antiviral agent may be accompanied by a withdrawal of administering the at least one second antiviral agent as well. Yet in some embodiments, administering the at least one second antiviral agent may continue for a third time period after the withdrawal of administering the at least one first antiviral agent. The third and the second time periods do not overlap, i.e. the administration of the at least one second antiviral agent without concurrent or sequential administering of the at least one first antiviral agent starts after the end of the second time period.

In some embodiments, the at least one second antiviral agent administered during the third time period may be the same as the at least one second antiviral agent administered during the second time period.

A duration of the third time period may vary. In some embodiments, the duration of the third time period may be at least about 1 week or at least about 2 weeks or at least about 4 weeks or at least about 12 weeks or at least about 18 weeks or at least about 24 weeks or at least about 30 weeks or at least about 36 weeks or at least about 40 weeks or at least about 48 weeks or at least about 60 weeks. In some embodiments, the third time period may last more than 60 weeks.

### Formulations and Routes of Administration

An active agent (e.g., any antiviral agent contained in the at least one first antiviral agent or in at least one second antiviral agent) is administered to individuals in a formulation with a pharmaceutically acceptable excipient(s). The terms “active agent” and “therapeutic agent” are used interchangeably herein. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20.sup.th edition, Lippincott, Williams, & Wilkins; Pharmaceutical Dosage Forms and Drug Delivery Systems (1999) H. C. Ansel et al., eds., 7.sup.th ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

An active agent may be administered to the host using any convenient means capable of resulting in the desired therapeutic effect. Thus, an active agent may be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent may be achieved in various ways, including oral, buccal, rectal, parenteral, intraperitoneal, intradermal, subcutaneous, intramuscular, transdermal, intratracheal, etc., administration. In some embodiments, two or more different routes of administration are used. For example, in some embodiments, an alpha-glucosidase inhibitor may be administered orally, while IFN-γ or IFN-α can be administered subcutaneously.

Subcutaneous administration of an active agent may be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, an active agent may be delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system may be an at least partially implantable device. The implantable device may be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally used because of convenience in implantation and removal of the drug delivery device.

Drug release devices suitable for use in the invention may be based on any of a variety of modes of operation. For example, the drug release device may be based upon a diffusive system, a convective system, or an erodible system (e.g., an erosion-based system). For example, the drug release device may be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device may be based upon an electrodiffusion system, an electrolytic pump, an effervescent pump, a piezoelectric pump, a hydrolytic system, etc.

Drug release devices based upon a mechanical or electromechanical infusion pump may be also suitable. Examples of such devices include those described in, for example, U.S. Pat. Nos. 4,692,147; 4,360,019; 4,487,603; 4,360,019; 4,725,852, and the like. In general, a subject treatment method can be carried out using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in a subject treatment method include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device may be implanted at any suitable implantation site using methods and devices well known in the art. As noted above, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that may be adapted for the present invention is the Synchromed infusion pump (Medtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

The agents may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent may be formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

Furthermore, an active agent may be made into suppositories by mixing with a variety of bases such as emulsifying bases or water-soluble bases. An active agent may be administered rectally via a suppository. The suppository may include vehicles such as cocoa butter, carbowaxes and polyethylene glycols, which melt at body temperature, yet are solidified at room temperature.

Unit dosage forms for oral or rectal administration, such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the agent(s) in a composition as a solution in sterile water, normal saline or another pharmaceutically acceptable carrier.

### Dosages

In some embodiments, the first administration procedure and the second administration procedure may involve administering a Type I interferon receptor agonist. A dosage of the Type I interferon receptor agonist administered during the first time period may be the same or different from the dosage of the Type I interferon agonist administered during the second time period. In many embodiments, the Type I interferon receptor agonist may be an IFN-α. Type I interferon receptor agonists suitable for use herein include any interferon-α (IFN-α). In certain embodiments, the interferon-α is a PEGylated interferon-α. In certain other embodiments, the interferon-α is a consensus interferon, such as INFERGEN® interferon alfacon-1. In still other embodiments, the interferon-α is a monoPEG (30 kD, linear)-ylated consensus interferon.

Effective dosages of an IFN-α may range from about 1 μg to about 3 μg, from about 3 μg to about 27 μg, from about 3 MU to about 10 MU, from about 90 μg to about 180 μg, or from about 18 μg to about 90 μg. Effective dosages of Infergen® consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFNα2b range from 3 million Units (MU) to 10 MU per dose. Effective dosages of PEGASYS®PEGylated IFN-α2a contain an amount of about 90 μg to 270 μg, or about 180 μg, of drug per dose. Effective dosages of PEG-INTRON®PEGylated IFN-®2b contain an amount of about 0.5 μg to 3.0 μg of drug per kg of body weight-per dose. Effective dosages of PEGylated consensus interferon (PEG-CIFN) may contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN may contain an amount of about 45 μg to about 270 μg, or about 60 μg to about 180 μg, or about 90 μg to about 120 μg, of drug per dose. IFN-α may be administered daily, every other day, once a week, three times a week, every other week, three times per month, once monthly, substantially continuously or continuously.

Dosage regimens for administering the Type I interferon receptor agonist may include tid, bid, qd, qod, biw, tiw, qw, qow, three times per month, or monthly administrations. In some embodiments, any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration may be provided. In other embodiments, any of the above-described methods in which the desired dosage of PEGylated IFN-α (PEG-IFN-α) is administered subcutaneously to the patient by bolus delivery qw, qow, three times per month, or monthly for the desired treatment duration may be provided.

In some embodiments, the first administration procedure and the second administration procedure may involve administering a Type II interferon receptor agonist. A dosage of the Type II interferon receptor agonist administered during the first time period may be the same or different from the dosage of the Type II interferon agonist administered during the second time period. In many embodiments, the Type II interferon agonist may be an IFN-γ.

Effective dosages of IFN-γ may range from about 0.5 μg/m² to about 500 μg/m², usually from about 1.5 μg/m² to 200 μg/m², depending on the size of the patient. This activity is based on 10⁶ international units (U) per 50 μg/m² of protein. IFN-γ may be administered daily, every other day, three times a week (tiw), or substantially continuously or continuously. In specific embodiments, IFN-γ may be administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments, IFN-γ1b may be administered. In some embodiments, the IFN-γ may be Actimmune® human IFN-γ1b.

Where the dosage is 200 μg IFN-γ per dose, the amount of IFN-γ per body weight (assuming a range of body weights of from about 45 kg to about 135 kg) may be in the range of from about 4.4 μg IFN-γ per kg body weight to about 1.48 μg IFN-γ per kg body weight. The body surface area of individuals to be treated generally may range from about 1.33 m² to about 2.50 m². Thus, in many embodiments, an IFN-γ dosage may range from about 150 μg/m² to about 20 μg/m². For example, an IFN-γ dosage may range from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups may range from about 25 μg/m² to about 100 μg/m². In other embodiments, the dosage groups may range from about 25 μg/m² to about 50 μg/m².

In many embodiments, multiple doses of an IFN-γ may be administered. For example, an IFN-γ may be administered once per month, twice per month, three times per month, every other week (qow), once per week (qw), twice per week (biw), three times per week (tiw), four times per week, five times per week, six times per week, every other day (qod), daily (qd), substantially continuously, or continuously.

In some embodiments, the first administration procedure and the second administration procedure may include administering ribavirin. A dosage of ribavirin administered during the first time period may be the same or different from a dosage of ribavirin administered during the second time period. Ribavirin may be administered in dosages ranging from about 20 mg/day to about 1500 mg/day, such as about 200 mg/day, about 400 mg/day, about 800 mg/day, about 1000 mg/day or about 1200 mg/day. In some embodiments, ribavirin may be administered orally in dosages randing from about 800 mg/day to about 1200 mg/day.

In some embodiments, the first administration procedure and the second administration procedure may include administering levovirin. A dosage of levovirin administered during the first time period may be the same or different from a dosage of levovirin administered during the second time period. Levovirin may be administered in an amount ranging from about 30 mg to about 60 mg, from about 60 mg to about 125 mg, from about 125 mg to about 200 mg, from about 200 mg to about 300 mg, from about 300 mg to about 400 mg, from about 400 mg to about 1200 mg, from about 600 mg to about 1000 mg, or from about 700 to about 900 mg per day, or about 10 mg/kg body weight per day. In some embodiments, levovirin may be administered orally in dosages of about 400 mg, about 800 mg, about 1000 mg, or about 1200 mg per day.

In some embodiments, the first administration procedure and the second administration procedure may include administering viramidine. A dosage of viramidine administered during the first time period may be the same or different from a dosage of viramidine administered during the second time period. Viramidine may be administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day. In some embodiments, viramidine may be administered orally in dosages of about 800 mg, or about 1600 mg per day.

In some embodiments, the first administration procedure and the second administration procedure may include administering thymosin-α. A dosage of thymosin-α administered during the first time period may be the same or different from a dosage of thymosin-a administered during the second time period. Thymosin-α (Zadaxin™) may be administered by subcutaneous injection. Thymosin-α may be administered tid, bid, qd, qod, biw, tiw, qw, qow, three times per month, once monthly, substantially continuously, or continuously. In many embodiments, thymosin-α may be administered twice per week.

Effective dosages of thymosin-α may range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In some embodiments, thymosin-α may be administered in dosages containing an amount of 1.0 mg or 1.6 mg.

In some embodiments, the first administration procedure and the second administration procedure may include administering an HCV enzyme inhibitor. A dosage of the HCV enzyme inhibitor administered during the first time period may be the same or different from a dosage of the HCV enzyme inhibitor administered during the second time period. In some embodiments, the HCV enzyme inhibitor may be an NS3 inhibitor, yet in some embodiments, the HCV enzyme inhibitor may be an NS5 inhibitor.

Effective dosages of an HCV enzyme inhibitor may range from about 10 mg to about 200 mg per dose, e.g., from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, from about 35 mg to about 40 mg per dose, from about 40 mg per dose to about 45 mg per dose, from about 45 mg per dose to about 50 mg per dose, from about 50 mg per dose to about 60 mg per dose, from about 60 mg per dose to about 70 mg per dose, from about 70 mg per dose to about 80 mg per dose, from about 80 mg per dose to about 90 mg per dose, from about 90 mg per dose to about 100 mg per dose, from about 100 mg per dose to about 125 mg per dose, from about 125 mg per dose to about 150 mg per dose, from about 150 mg per dose to about 175 mg per dose, or from about 175 mg per dose to about 200 mg per dose.

In some embodiments, effective dosages of an HCV enzyme inhibitor may be expressed as mg/kg body weight. In these embodiments, effective dosages of an HCV enzyme inhibitor may range from about 0.01 mg/kg body weight to about 100 mg/kg body weight, from about 0.1 mg/kg body weight to about 50 mg/kg body weight, from about 0.1 mg/kg body weight to about 1 mg/kg body weight, from about 1 mg/kg body weight to about 10 mg/kg body weigh, from about 10 mg/kg body weight to about 100 mg/kg body weight, from about 5 mg/kg body weight to about 400 mg/kg body weight, from about 5 mg/kg body weight to about 50 mg/kg body weight, from about 50 mg/kg body weight to about 100 mg/kg body weight, from about 100 mg/kg body weight to about 200 mg/kg body weight, from about 200 mg/kg body weight to about 300 mg/kg body weight, or from about 300 mg/kg body weight to about 400 mg/kg body weight.

The HCV enzyme inhibitor may be administered tid, bid, qd, qod, biw, tiw, qw, qow, three times per month, once monthly, substantially continuously, or continuously.

In some embodiments, for treating an HCV viral infection, the first administration procedure and the second administration procedure may include administering an HCV NS3 protease inhibitor containing an amount of 0.01 mg to 100 mg of drug per kilogram of body weight orally daily, optionally in two or more divided doses per day.

In some embodiments, for treating an HCV viral infection, the first administration procedure and the second administration procedure may include administering an HCV NS5B RNA-dependent RNA polymerase inhibitor containing an amount of 0.01 mg to 100 mg of drug per kilogram of body weight orally daily, optionally in two or more divided doses per day.

In some embodiments, the second administration procedure and optionally the third administration procedure, i.e. administering procedure performed during the third time period after the end of the second time period, may involve administering α-glucosidase inhibitor. A dosage of the α-glucosidase inhibitor administered during the second time period may be the same or different from a dosage of the α-glucosidase inhibitor optionally administered during the third time period.

In some embodiments, α-glucosidase inhibitor may be administered to the patient at a dosage of from about 1 mg per day to about 600 mg per day in divided doses, e.g., from about 30 mg per day to about 60 mg per day, from about 60 mg per day to about 75 mg per day, from about 75 mg per day to about 90 mg per day, from about 90 mg per day to about 120 mg per day, from about 120 mg per day to about 150 mg per day, from about 150 mg per day to about 180 mg per day, from about 180 mg per day to about 210 mg per day, from about 210 mg per day to about 240 mg per day, from about 240 mg per day to about 270 mg per day, from about 270 mg per day to about 300 mg per day, from about 300 mg per day to about 360 mg per day, from about 360 mg per day to about 420 mg per day, from about 420 mg per day to about 480 mg per day, or from about 480 mg to about 600 mg per day.

In some embodiments, the dosage of the α-glucosidase inhibitor may be expressed in mg/kg of body weight. As such, the dosage of the α-glucosidase inhibitor may range from about 0.01 mg/kg/day to about 2500 mg/kg/day or from about 0.1 mg/kg/day to about 200 mg/kg/day or from about 1 mg/kg/day to about 100 mg/kg/day or from about 1 mg/kg/day to about 5 mg/kg/day or from about 5 mg/kg/day to about 20 mg/kg/day.

In some embodiments, the second administration procedure and optionally the third administration procedure, i.e. administering procedure performed during the third time period after the end of the second time period, may involve administering N-butyl deoxynojirimycin. A dosage of the α-glucosidase inhibitor administered during the second time period may be the same or different from a dosage of N-butyl deoxynojirimycin optionally administered during the third time period.

In some embodiments, N-butyl deoxynojirimycin may be administered to the patient at a dosage of from about 1 mg per day to about 600 mg per day in divided doses, e.g., from about 30 mg per day to about 60 mg per day, from about 60 mg per day to about 75 mg per day, from about 75 mg per day to about 90 mg per day, from about 90 mg per day to about 120 mg per day, from about 120 mg per day to about 150 mg per day, from about 150 mg per day to about 180 mg per day, from about 180 mg per day to about 210 mg per day, from about 210 mg per day to about 240 mg per day, from about 240 mg per day to about 270 mg per day, from about 270 mg per day to about 300 mg per day, from about 300 mg per day to about 360 mg per day, from about 360 mg per day to about 420 mg per day, from about 420 mg per day to about 480 mg per day, or from about 480 mg to about 600 mg per day.

In some embodiments, the dosage of the N-butyl deoxynojirimycin may be expressed in mg/kg of body weight. As such, the dosage of N-butyl deoxynojirimycin inhibitor may range from about 0.01 mg/kg/day to about 2500 mg/kg/day or from about 0.1 mg/kg/day to about 200 mg/kg/day or from about 1 mg/kg/day to about 100 mg/kg/day or from about 1 mg/kg/day to about 5 mg/kg/day or from about 5 mg/kg/day to about 20 mg/kg/day.

In some embodiments, the second administration procedure and optionally the third administration procedure, i.e. administering procedure performed during the third time period after the end of the second time period, may involve administering an inhibitor of ion channel activity. A dosage of the inhibitor of ion channel activity administered during the second time period may be the same or different from a dosage of the inhibitor of ion channel activity optionally administered during the third time period.

In some embodiments, the dosage of the ion channel activity inhibitor may range from about 0.01 mg/kg/day to about 1000 mg/kg/day or from about 0.1 mg/kg/day to about 100 mg/kg/day or from about 1 mg/kg/day to about 1 mg/kg/day to 10 mg/kg/day or from about 5 mg/kg/day to about 50 mg/kg/day.

In some embodiments, the second administration procedure and optionally the third administration procedure, i.e. administering procedure performed during the third time period after the end of the second time period, may involve administering a compound of formula VIII. A dosage of the compound of formula VIII administered during the second time period may be the same or different from a dosage of the compound of formula VII administered during the third time period.

In some embodiments, the dosage of the compound of formula VIII may range from about 0.01 mg/kg/day to about 1000 mg/kg/day or from about 0.1 mg/kg/day to about 100 mg/kg/day or from about 1 mg/kg/day to about 1 mg/kg/day to 10 mg/kg/day or from about 5 mg/kg/day to about 50 mg/kg/day.

### Patients Selection

In certain embodiments, the specific at least one first antiviral agent and the duration of the first period administered to the subject may depend on parameters of the viral infection exhibited in the subject. For example, for the HCV viral infection such parameters may include the initial viral load in the subject, genotype of the HCV infection in the subject, liver histology and/or stage of liver fibrosis in the subject.

In some embodiments, for a naïve patient infected with HCV genotype I virus with an HCV viral load of greater than 2×10⁶ HCV genome, the at least one first antiviral agent may include peginterferon-α2a and a dose of ribavirin of at least about 1000 mg/day, such as 1000 mg/day or 1200 mg/day, and the first time period may be about 48 weeks.

The subject methods are suitable for treating individuals having, or susceptible to having, an alphavirus infection, e.g., a flavivirus infection (e.g., an HCV infection, etc.). In many embodiments, the individual is a human.

Individuals who have been clinically diagnosed as infected with an alphavirus are suitable for the treatment methods. Of particular interest in some embodiments are individuals who have been clinically diagnosed as infected with a hepatitis virus (e.g., HAV, HBV, HCV, delta, etc.).

Individuals who are to be treated according to the present methods may include individuals who have been clinically diagnosed as infected with HCV. Individuals who are infected with HCV may be identified as having a detectable level of HCV RNA in their blood, and/or having anti-HCV antibody in their serum.

Individuals who are clinically diagnosed as infected with HCV may include naive individuals (e.g., individuals not previously treated for HCV, particularly those who have not previously received IFN-α-based and/or ribavirin-based therapy) and individuals who have failed prior treatment for HCV (“treatment failure” patients). Treatment failure patients may include non-responders (i.e., individuals in whom the HCV titer was not significantly or sufficiently reduced by a previous treatment for HCV, such as a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In some embodiments, treated individuals may have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype, such as HCV genotype 1 and particular HCV subtypes and quasispecies.

In some embodiments, HCV-positive individuals suitable for treatment may exhibit severe fibrosis or early cirrhosis (non-decompensated, Child's-Pugh class A or less), or more advanced cirrhosis (decompensated, Child's-Pugh class B or C) due to chronic HCV infection. In particular embodiments, HCV-positive individuals with stage 3 or 4 liver fibrosis according to the METAVIR scoring system are suitable for treatment with the disclosed methods. In other embodiments, individuals suitable for treatment with the disclosed methods are patients with decompensated cirrhosis with clinical manifestations, including patients with far-advanced liver cirrhosis, including those awaiting liver transplantation. In still other embodiments, individuals suitable for treatment with the disclosed methods include patients with milder degrees of fibrosis including those with early fibrosis (stages 1 and 2 in the METAVIR, Ludwig, and Scheuer scoring systems; or stages 1, 2, or 3 in the Ishak scoring system.). Various fibrosis scoring systems are known to those skilled in the art and are detailed, for example, in paragraphs 0092-0097 of US patent publication No. 2006/0269517.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### Example 1

MDBK cells were infected with non-cytopathic (ncp) BVDV strain Pe515 at a MOI of 0.1 and passaged with fresh medium every three days. After 6 passages a stable infection was achieved. IFN (1000 U) and RBV (2 μM) were then added to the cells; this passage was denoted passage 1 (P1). In addition, mock-infected negative controls were set up in the presence and absence of IFN (1000 U) and RBV (2 μM). Cells were passaged every 3 days with a 1:8 dilution into fresh medium containing drugs. At passage 3 (P3) the medium was supplemented with iminosugars and the cells cultured in the presence or absence of different concentrations of NB-DNJ (10, 50 and 100 μM), 100 μM 231B and 50 μM NN-DNJ. The cells were passaged every three days with fresh medium containing drugs. After a further 9 passages (i.e. at passage 12) each sample was split into the following three sets: set 1, where all drug combinations remained the same and the cells were cultured in the presence of IFN/RBV and the iminosugars at the stated concentrations; set 2, where all drugs were removed; and set 3, where only IFN/RBV were removed i.e. the cells were cultured in the presence of the iminosugars. Cells were passaged every three days as described previously for a further ten passages (i.e. P12-22). At each passage the supernatants of cultured cells were harvested and analyzed for RNA copies (by real time RT-PCR) and the ability to infect naïve MDBK cells (by immunofluorescence, IF). Three days after the final passage (P22) cells were probed for the presence of BVDV within the cells by IF.

### Infectivity Assays Using Immunofluorescence Analysis

The ability of cultured cells treated or not with the various drug combinations to infect naïve MDBK cells was determined using immunofluorescence microscopy. Naïve MDBK cells were grown in six-well plates to 70% confluency and the supernatant was removed and discarded. Cells were infected with 500 μl of the harvested supernatant from the mock-infected and BVDV-infected cells for 1 h at 37° C. The inoculum was removed, the cells washed twice with Phosphate Buffered Saline (PBS) and incubated in RPMI 1640 medium containing 10% (v/v) FCS overnight. The supernatant was removed and cells were fixed using 2% paraformaldehyde for 30 minutes. Cells were washed with PBS, blocked in 5% (w/v) milk/PBS solution for 30 minutes, and then permeabilised using 1% Triton X-100 for 20 minutes. Cells were washed with 1% (v/v) Tween®/PBS and incubated for 1 hr with a monoclonal antibody against the BVDV NS2/NS3 proteins. BVDV-infected cells were detected by probing with an anti-mouse-fluorescein isothiocyanate (FITC)-conjugated secondary antibody.

### Viral RNA Purification and Real Time RT-PCR Analysis

The number of viral RNA copies in cells treated or not with the various drug combinations were determined by real-time RT-PCR. RNA from the harvested supernatants from mock-infected and BVDV-infected cells was concentrated, purified and treated with DNAse. Real time RT-PCR was then performed to determine the number of BVDV viral RNA copies present in the supernatants from the cultured cells.

### Results

### Establishment of a MDBK Cell Line Persistently Infected with ncp BVDV

To study the ability of cocktails of antiviral compounds to eliminate BVDV infection, a MDBK cell line persistently infected with ncp BVDV was established. Analysis by immunofluorescence microscopy showed that 90% of the cells were infected with BVDV.

### Antiviral Effect of IFN and RBV in Combination with Iminosugars on Persistent BVDV Infection in MDBK Cells

Having attained a stable infection, IFN (1000 U) and RBV (2 μM) were added to the cells (referred from herein as passage 1; P1). Cells were maintained in medium containing 10% BVDV-free FCS with or without drug and passaged every 3 days with a 1:8 dilution. The levels of viral RNA present in the supernatant during successive passages were monitored by real time RT-PCR and IF. IFN/RBV treatment caused a decrease in viral RNA and BVDV infectivity to non detectable levels after 3 passages, confirming the antiviral properties of IFN/RBV in combination. The supernatant from the non-drug-treated control sample contained 27000 RNA copies per ml of medium and caused an infection level of 13%, as measured by RT-PCR and IF, respectively.

From passage 3 the infected MDBK cell line was cultured in the presence of IFN/RBV supplemented with different concentrations of the iminosugars, 10, 50 and 100 μM NB-DNJ; 100 μM 231B; and 50 μM NN-DNJ. After a further 9 passages (i.e. at passage 12) under this drug pressure, each sample was split into the following three sets: set 1, where all drug combinations remained the same and cells were cultured in the presence of the IFN/RBV/iminosugar triple cocktail; set 2, where all drugs were removed; and set 3, where IFN/RBV were removed, i.e. cells were cultured in the presence of the iminosugars only. Viral RNA levels were monitored by real time-RT-PCR and infectivity assays were performed at each passage to monitor the effects of the different drug combinations. Following removal (or not) of the drugs the cells were monitored for a further 10 passages (passages 12-22; 30 days).

In set 1, where all drugs were left on, viral RNA was detected only in non-drug-treated samples (Table 1). By the conclusion of the experiment, the cells had been treated with IFN/RBV for 22 passages and no breakthrough of viral signal occurred, indicating that no resistant virus was present.

TABLE 1

| | Mean copy | |
|---|---|---|
| Set 1 | number | % of Control |
| No Drug | 14518.1 | 100 |
| I/R | 0.5676125 | 0 |
| Mock | 0 | 0 |
| 10 NB | 0 | 0 |
| 50 NB | 0 | 0 |
| 100 NB | 0 | 0 |
| 50 NN | 0 | 0 |
| 100 231B | 0 | 0 |

Table 1 presents viral RNA copies detected at passage 22 by real-time RT-PCR in supernatant harvested from MDBK cells persistently infected with BVDV for sample set 1. Cells were treated for passages 3-22 in the absence of drug (no drug), in the presence of IFN/RBV only (I/R), or with triple combinations of IFN/RBV/10 μM NB-DNJ (10 NB), IFN/RBV/50 μM NB-DNJ (50 NB), IFN/RBV/100 μM NB-DNJ (100 NB), IFN/RBV/50 μM NN-DNJ (50 NN), or IFN/RBV/100 μM 231B (100 231B). Mock-infected cells (Mock) were also analysed. With the exception of the cells treated in the absence of drug, no viral RNA was detected.

Samples in set 2 were those cultured in the absence of all drugs for P12-22 after treatment for 9 passages (P3-12) on the various IFN/RBV/iminosugar triple cocktail combinations. No virus was detected in any of these samples during P12-22 (as measured by real-time RT-PCR, Table 2). However, for those samples that had been treated with IFN/RBV, as a double cocktail only for P3-12 (i.e. no iminosugars), viral rebound was observed. The infectivity of the secreted virus was assayed by IF (Table 4). In support of these data, no infectious virus was detected in those samples that had been treated with the various IFN/RBV/iminosugar triple cocktail combinations; however, BVDV infection was detected in those samples that had been treated with IFN/RBV only. These results indicate that the IFN/RBV/iminosugar combination successfully eradicated the virus from the persistently infected cell line;

TABLE 2

| Set 2 | Mean copy number | % of Control |
|---|---|---|
| No Drug | 18300.9735 | 100 |
| I/R | 15784 | 86.24678 |
| Mock | 0 | 0 |
| 10 NB | 0 | 0 |
| 50 NB | 0 | 0 |
| 100 NB | 0 | 0 |
| 50 NN | 0 | 0 |
| 100 231B | 0 | 0 |

Table 2 presents viral RNA copies detected at passage 22 by real-time RT-PCR in supernatant harvested from MDBK cells persistently infected with BVDV for sample set 2. Cells were treated for passages 3-12 in the absence of drug (no drug), in the presence of IFN/RBV only (I/R), or with triple combinations of IFN/RBV/10 μM NB-DNJ (10 NB), IFN/RBV/50 μM NB-DNJ (50 NB), IFN/RBV/100 μM NB-DNJ (100 NB), IFN/RBV/50 μM NN-DNJ (50 NN), or IFN/RBV/100 μM 231B (100 231B). Mock-infected cells (Mock) were also analyzed. At passage 12, all drug pressure was removed and the cells were cultured for a further 10 passages (P12-22). After the removal of drug pressure no viral RNA was detected in samples that had been treated with a triple combination of IFN/RBV/iminosugar.

For sample set 3, that is the samples that were cultured in the presence of the iminosugars only for P12-22 after treatment for 9 passages on the various IFN/RBV/iminosugar triple cocktail combinations, the results were as for set 2. That is, no virus rebound was seen in those samples that had been treated with the IFN/RBV/iminosugar triple cocktail combinations (Table 3 and Table 4). As for set 2, here BVDV virus was detected in those samples that had been treated only with the IFN/RBV combination. Again, these results indicate that the IFN/RBV/iminosugar combination successfully eradicated the virus from the persistently infected cell line.

TABLE 3

| Set 3 | Mean copy number | % of Control |
|---|---|---|
| No Drug | 33445.55 | 100 |
| I/R | 46665.05 | 139.5254 |
| Mock | 0 | 0 |
| 10 NB | 0 | 0 |
| 50 NB | 0 | 0 |
| 100 NB | 0 | 0 |
| 50 NN | 0 | 0 |
| 100 231B | 0 | 0 |

Table 3 presents viral RNA copies detected at passage 22 by real-time RT-PCR in supernatant harvested from MDBK cells persistently infected with BVDV for sample set 3. Cells were treated for passages 3-12 in the absence of drug (no drug), in the presence of IFN/RBV only (I/R), or with triple combinations of IFN/RBV/10 μM NB-DNJ (10 NB), IFN/RBV/50 μM NB-DNJ (50 NB), IFN/RBV/100 μM NB-DNJ (100 NB), IFN/RBV/50 μM NN-DNJ (50 NN), or IFN/RBV/100 μM 231B (100 231B). Mock-infected cells (Mock) were also analysed. At passage 12, IFN/RBV drug pressure was removed and the cells were cultured for a further 10 passages in the presence of the iminosugars at the stated concentrations (P12-22). After the removal of the IFN/RBV drug pressure no viral RNA was detected in samples that had been treated with a triple combination of IFN/RBV/iminosugar.

Together, these data indicate that treatment for 9 passages with the IFN/RBV/NB-DNJ, or IFN/RBV/NN-DNJ or IFN/RBV/231B triple cocktail combinations is able to clear the BVDV infection from a persistently infected MDBK cell line, even after the removal of all drug pressure. No viral RNA was detected at passage 22 by real-time RT-PCR (Tables 1-3) nor were any infected cells detected by IF (Table 4) in all those samples that had been treated for passages 3-12 on the IFN/RBV/iminosugar triple cocktail, even after the removal of all (set 2) or just the IFN/RBV (set 3) drug pressure.

Table 4 presents percentage of naïve cells infected by incubation with supernatant harvested from MDBK cells persistently infected with BVDV for 1 hour, as detected by IF. Data shown are for passage 22 (P22) only, for sample sets 1 (S1), 2 (S2) and 3 (S3). Cells were treated for passages 3-12 in the absence of drug (no drug), in the presence of IFN/RBV only (I/R), or with triple combinations of IFN/RBV/100 μM 231B (100 231B), IFN/RBV/10 μM NB-DNJ (10 NB), IFN/RBV/50 μM NB-DNJ (50 NB), IFN/RBV/100 μM NB-DNJ (100 NB). Mock-infected cells and mock-infected cells treated with IFN/RBV were also analysed. At passage 12, samples were divided into three sets: S1 where all drugs remained on, S2 where all drugs were removed and S3 where IFN/RBV drug pressure was removed and the cells were cultured for a further 10 passages in the presence of the iminosugars at the stated concentrations.

TABLE 4

| | S1 P22 | S2 P22 | S3 P22 |
|---|---|---|---|
| No Drug | 9.5 | 9.5 | 9.5 |
| I/R | 0 | 15 | 10 |
| 100 231B | 0 | 0 | 0 |
| 10 NB | 0 | 0 | 0 |
| 50 NB | 0 | 0 | 0 |
| 100 NB | 0 | 0 | 0 |
| Mock-infected | 0 | 0 | 0 |
| Mock-infected + I/R | 0 | 0 | 0 |

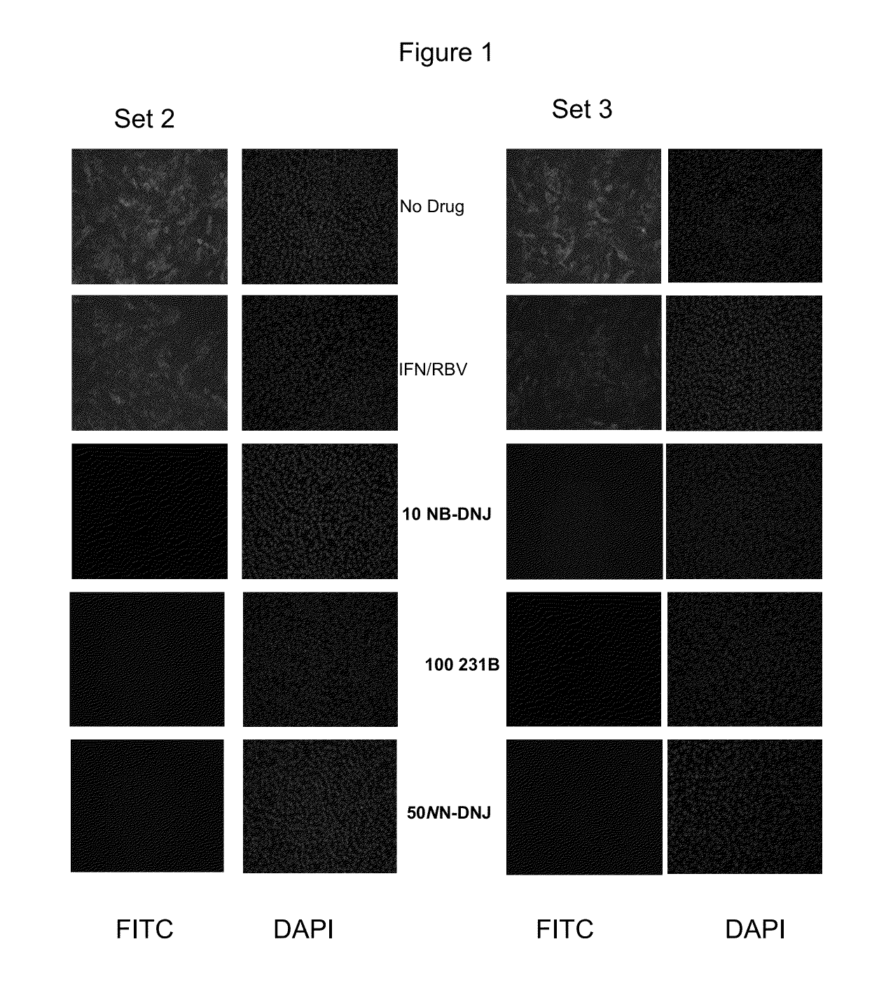

To confirm clearance of the virus, three days after the final passage (P22) cells were probed for the presence of BVDV within the cells by IF (FIG. 1). In all sample sets BVDV was detected in non-drug-treated cells. Additionally, in sets 2 and 3 BVDV was detected in samples that had been treated with IFN/RBV only. BVDV was not detected in cells in sets 2 and 3 that had been treated with the triple cocktails of IFN/RBV/NB-DNJ, IFN/RBV/NN-DNJ or IFN/RBV/100 μM 231b. These results show that, under the experimental conditions used, the triple cocktails were able to clear BVDV infection from a persistently infected MDBK cell line. Moreover, no rebound of virus was observed during any of the 22 passages under IFN/RBV pressure, indicating that any potentially resistant virus was kept in check for the time frame of this experiment.

### Conclusion

By culturing the infected cells in the presence of the triple drug cocktail for nine passages (P3-12), BVDV viral RNA was undetectable even after the drugs were removed. Thus, IFN/RBV/iminosugar combination successfully eradicated the virus from the persistently infected cell line; as supported and confirmed by the results of the infectivity assay.

### Example 2

As in the example 1, MDBK cells were infected with non-cytopathic (ncp) BVDV strain Pe515 at a MOI of 0.1 and passaged with fresh medium every three days. After 6 passages a stable infection was achieved. IFN (1000 U) and RBV (2 μM) were then added to the cells; this passage was denoted passage 1 (P1). In addition, mock-infected negative controls were set up in the presence and absence of IFN (1000 U) and RBV (2 μM). Cells were passaged every 3 days with a 1:8 dilution into fresh medium containing drugs. At passage 3 (P3) the medium was supplemented with NB-DNJ and the cells cultured in the presence or absence of different concentrations of NB-DNJ (0.1, 1 and 10 μM). The cells were passaged every three days with fresh medium containing drugs. After a further 9 passages (i.e. at passage 12) each sample was split into the following three sets: set 1, where all drug combinations remained the same and the cells were cultured in the presence of IFN/RBV and NB-DNJ at the stated concentrations; set 2, where both IFN/RBV and NB-DNJ were removed; and set 3, where only IFN/RBV were removed, i.e. the cells were cultured in the presence of NB-DNJ only. Cells were passaged every three days as described previously for a further ten passages (i.e. P12-22). At each passage the supernatants of cultured cells were harvested and analyzed for RNA copies (by real time RT-PCR) and the ability to infect naïve MDBK cells (by immunofluorescence, IF). Three days after the final passage (P22) cells were probed for the presence of BVDV within the cells by IF.

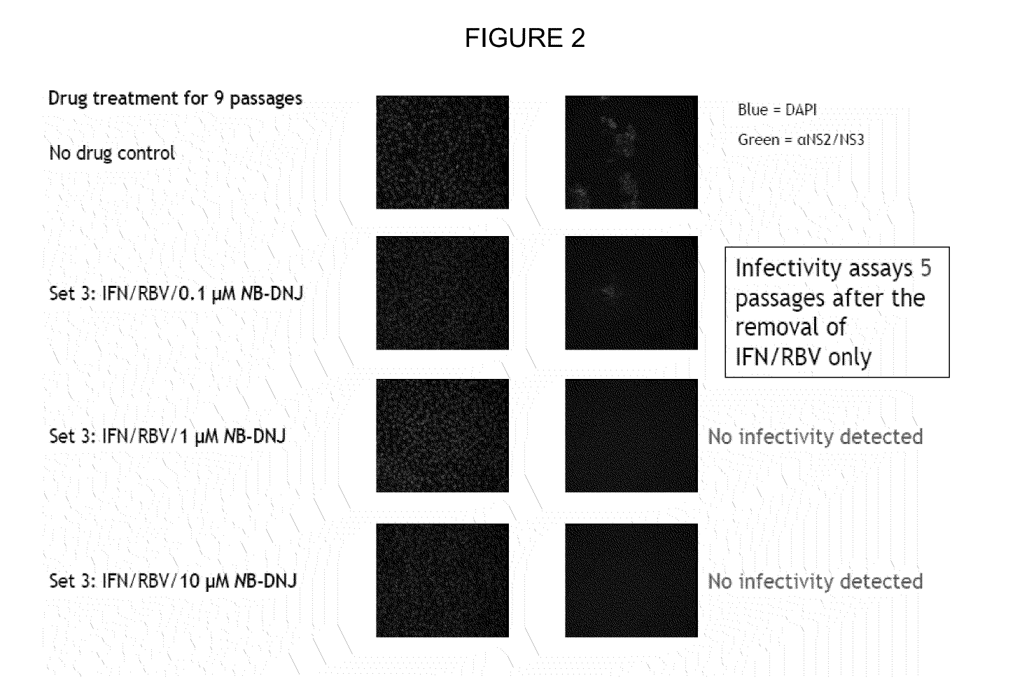

FIG. 2 presents results of detection of ncp BVDV in the persistently infected MDBK cells by IF five passages after removal of interferon and ribavirin only while maintaining NB-DNJ treatment. Data are shown for cells treated for passages 3-12 in the absence of drug (no drug), in the presence of IFN/RBV only (I/R), or with triple combinations of IFN/RBV/0.1 μM NB-DNJ, IFN/RBV/1 μM NB-DNJ, IFN/RBV/10 μM NB-DNJ. The data in FIG. 2 show that no relapse of the infection is detected in cells maintained at either 1 μM NB-DNJ or 10 μM NB-DNJ five passages after removal of interferon and ribavirin.

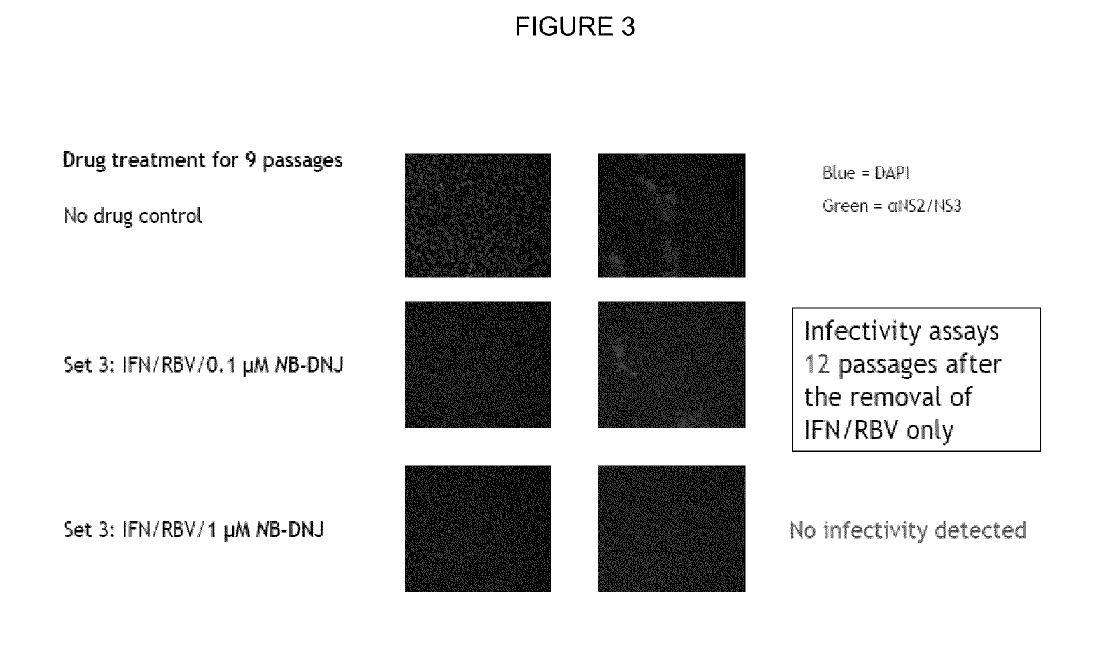

FIG. 3 presents results of detection of ncp BVDV in the persistently infected MDBK cells by IF twelve passages after removal of interferon and ribavirin only while maintaining NB-DNJ treatment. Data are shown for cells treated for passages 3-12 in the absence of drug (no drug), or with triple combinations of IFN/RBV/0.1 μM NB-DNJ or IFN/RBV/1 μM NB-DNJ. The data in FIG. 3 show that no relapse of the infection is detected in cells maintained at 1 μM NB-DNJ twelve passages after removal of interferon and ribavirin.

### Example 3

Bovine viral diarrhea virus (BVDV) is often used as a surrogate model for human hepatitis C virus (HCV). As members of the same family (Flaviviridae), their genomic organisation, replication strategies and putative life cycles have many similarities [1], see References list infra. Prior to the development of a cell culture HCV (HCVcc) infectivity system, BVDV, as the closest related virus, was the preferred model system for studies which depend on the ability to recreate a whole infectious cycle in cell culture. Although most aspects of HCV morphogenesis, viral secretion and re-infection can now be studied in the HCVcc system, others remain problematic; most notably the long-term culture of HCV infected host cells. The latter is needed to enable the study of viral clearance, emergence of viral escape mutants and importantly, viral rebound after cessation of extended drug treatment, with the aim of mirroring or improving upon clinical observations. In this context, BVDV is currently still the only available model system. In the present study it was shown that, in contrast to interferon/ribavirin treatment alone, the inclusion of an iminosugar in a triple combination eradicates non-cytopathic (ncp) BVDV from persistently infected MDBK cells, and prevents viral rebound after treatment is stopped. It was shown in an optimised treatment regime that a successful outcome can be achieved using NB-DNJ drug concentrations which are achievable in human patient serum.

### Introduction

Chronic hepatitis in humans is often caused by persistent infection with hepatitis C virus (HCV). This persistent infection may commonly lead to liver cirrhosis and hepatocellular carcinoma. Pegylated alpha interferon (IFN) in combination with ribavirin (RBV) can be the current treatment of choice for HCV [2]. However, treatment outcome is viral genotype specific and not effective in up to 50% of cases (Feld, 2005 #89). New therapies are urgently required with the aim of total and permanent viral eradication. IFN and RBV were evaluated as a dual therapy and as part of triple combination therapies with one of the following iminosugars: N-butyl deoxynojirimycin (NB-DNJ), N-nonyl-DNJ (NN-DNJ) and N7-oxanonyl-6-deoxy-methyl-galactonojirimycin (N-7-DGJ). The ability of these compounds to clear bovine viral diarrhoea virus (BVDV), a surrogate model for HCV [3], [1], from a persistently infected MDBK cell line was determined by monitoring viral infectivity and secretion of viral RNA. It was demonstrated that a triple drug combination of IFN, RBV and an iminosugar can eradicate the BVDV infection in a time- and dose-dependent manner. Importantly, after extended treatment with the triple combination therapy it was observed a sustained virological response after removal of all three drugs. In contrast, for cells treated with IFN and RBV alone, viral rebound was observed after removal of these compounds. The generality of such approach was demonstrated by showing that each of the three iminosugars, NB-DNJ, NN-DNJ, and N7-DGJ, with differing modes of action, [4], [5], [6], [7], [8] in combination with IFN and RBV eradicate BVDV infection from persistently infected MDBK cells. Thus, the triple cocktail of IFN/RBV and an iminosugar may be of a greater therapeutic value for hepatitis C infection than IFN/RBV alone.

### Materials and Methods

### Treatment of ncp Infected MDBK Cells with IFN, RBV, and Iminosugar Derivatives

Madin-Darby bovine kidney cells (MDBK) MDBK cells were seeded at 1×10⁶ cells/35 mm dish, infected with non-cytopathic (ncp) BVDV strain Pe515 (National Animal Disease Laboratory) at a MOI of 0.1, and passaged into fresh RPMI 1640 medium containing 10% (v/v) Foetal calf serum using a 1:8 dilution every three days. After achieving a stable infection IFN (1000 IU) and RBV (1 μM) (Sigma-Aldrich) were added to the cells; this passage was denoted passage 0 (P0). Simultaneously, a non-drug treated positive control sample and mock-infected (M.I.) negative controls cultured in the presence and absence of IFN and RBV (1000 IU and 1 μM, respectively) were prepared. Cells continued to be passaged every 3 days into fresh medium containing IFN and RBV. At passage 3 (P3), the medium was supplemented with the various iminosugar derivatives and the cells cultured in the presence of IFN, RBV and NB-DNJ (Sigma-Aldrich) or N7-DGJ or NN-DNJ (United Therapeutics Corporation [Silver Spring, Md.]). The cells were passaged every three days into fresh medium containing drug combinations as indicated. After 5 passages under triple combination drug pressure (P8) each sample was split into three; set 1 where all drug combinations remained the same and the cells continued to be cultured in the presence of IFN/RBV and the iminosugars (continued triple combination); set 2 where all drugs were removed; and set 3 where IFN/RBV were removed, i.e. the cells continued to be cultured in the presence of the iminosugars only (iminosugar maintenance treatment). Cells were passaged as described above. At each passage the combined supernatants of cultured cells from duplicate wells were harvested (to account for biological variation), levels of secreted viral RNA measured (by real time RT-PCR using technical duplicates) and the infectivity of the supernatant determined using an immunofluorescence (IF) based infectivity assay.

Detection of BVDV within the Infected Cell Line.

To detect either the stable infection at the beginning of experiments or after the final passage after treatment, supernatants were harvested and the cells probed for the presence of BVDV. The persistently infected MDBK cells were fixed with 2% paraformaldehyde for 30 minutes. Cells were washed with PBS, blocked in 5% (w/v) milk/PBS solution for 30 minutes, and permeabilised using 1% Triton X-100 for 20 minutes. After washing with 1% (v/v) Tween/PBS, cells were incubated for 1 h with the primary antibody WB103/105 (1:500 dilution; Veterinary Laboratory Agency, Weybridge, U.K.), recognising the BVDV NS2/NS3 proteins, after subsequent incubation with an anti-mouse-fluorescein isothiocyanate (FITC)-conjugated secondary antibody (Sigma) and extensive washing in PBS, nuclei were stained with 4′,6-diamidino-2-phenylindole (DAPI) (Vector Laboratories Inc, CA, US). Fluorescence was observed under an inverted Nikon Eclipse TE200-U microscope.

### Infectivity Assays Using Immunofluorescence Analysis.

MDBK cells were grown in six-well plates to 70% confluency, the supernatant removed and discarded. Cells were infected for 1 h at 37° C. using 500 μl of the harvested supernatants from the BVDV-infected and mock-infected cells. After removal of the inoculum, cells were washed twice with PBS and incubated overnight in fresh medium. Infectivity was determined by IF as described above.

### Viral RNA Purification and Real Time RT-PCR Analysis.

At each passage, a 500 μl aliquot of each supernatant harvested from the cultured cells was concentrated by ultrafiltration (10 kDa molecular weight cut off Centricon filter; Millipore, Mass., USA) to 140 μl. RNA from released viral particles was purified from the concentrated supernatants using the QIAamp Viral RNA purification kit (Qiagen, Crawley, U.K) according to the manufacturer's instructions. Briefly, RNA was eluted in 50 μA and samples were DNAse treated (90 minutes at 37° C., 20 minutes at 80° C.). Real time RT-PCR was carried out using the Qiagen Quantitect RT-PCR kit. Primers amplifying a 334 by region spanning parts of the NS2 coding sequence were used (forward 5′ TAG GGC AAA CCA TCT GGA AG 3′, reverse 5′ ACT TGG AGC TAC AGG CCT CA 3′). Reverse transcription was achieved at 50° C. for 30 minutes followed by incubation at 95° C. for 15 minutes to activate the hot start polymerase. The resulting DNA was amplified by PCR (35 cycles of 15 s at 95° C., 1 minute at 50° C., and 1 minute at 72° C.; final extension for 7 minutes at 72° C.).

### MTS Cell Proliferation Assay.

Cellular toxicity was measured using the Cell Titre 96 Aqueous Non-Radioactive Cell Proliferation Assay Kit according to the manufacturer's instructions (Promega, Wis., USA). MTS/phenazine methosulphate (PMS) solution (40 μl) was added to each well and the samples incubated at 37° C. in a humidified 5% CO₂ atmosphere for 3 h. The absorbance was read at 490 nm using a UVmax plate reader (Molecular Devices). Each sample was analysed in triplicate.

### Results

Generation of an MDBK Cell Line Persistently Infected with ncp BVDV.

To study the ability of antiviral compounds to eliminate BVDV infection, an MDBK cell line persistently infected with ncp BVDV was prepared. MDBK cells were infected with ncp BVDV at a MOI of 0.1. Monitoring viral RNA by real time RT-PCR showed that a stable infection was established after six passages. At that time 95% of the cells were infected as determined by immunofluorescence microscopy.

Evaluation of the antiviral effect of IFN and RBV in combination with iminosugar derivatives in persistently infected MDBK cells.

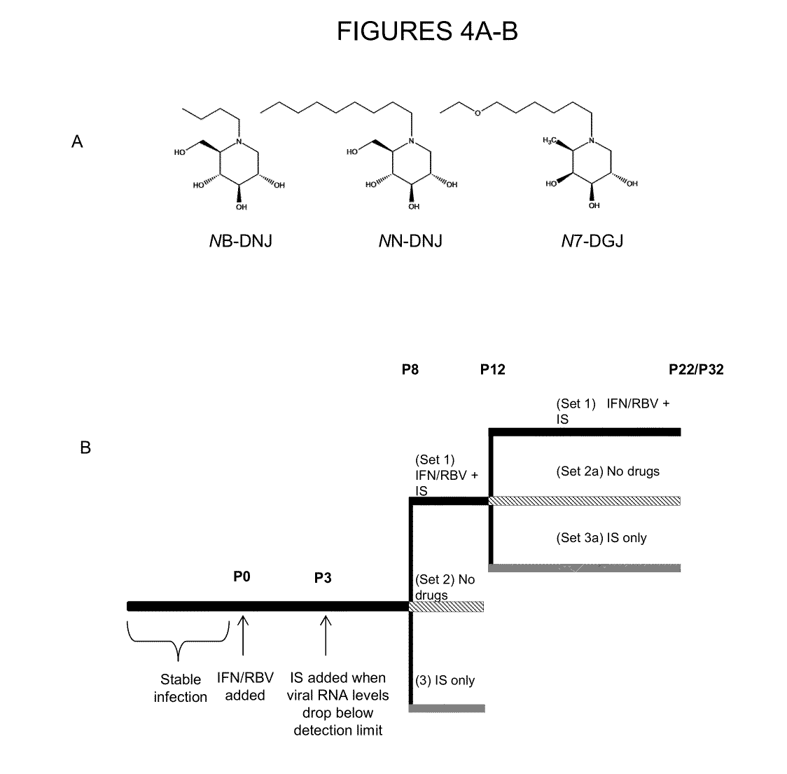

The chemical structures of the iminosugar derivatives used in this study and the experimental outline are shown in FIG. 4. The aim of this study was to evaluate whether the viral relapse observed after cessation of treatment with the IFN/RBV double combination could be delayed or prevented by the inclusion of an iminosugar derivative in a triple combination.

First, it was established that adding an iminosugar to the IFN/RBV double combination from the beginning did not cause viral RNA levels in the supernatants to decrease faster or in a synergistic fashion. Therefore, IFN/RBV alone were used to achieve the initial fast drop in measurable virus titres. Culturing persistently infected MDBK cells in the presence of IFN (1000 IU) and RBV (1 μM) for three passages (P0-P3) led to a decrease of viral RNA present in the supernatant to below the detection limit of real time RT-PCR, and inoculation of naïve MDBK cells with this supernatant did not lead to infection, as determined by IF. In contrast, supernatant taken from the untreated control sample contained 27000 RNA copies per ml and could re-infect naïve cells.

At passage 3, after viral titres had dropped to below detection limits in IFN/RBV treated samples, the cells were cultured for either an additional five (P3-P8) or nine passages (P3-P12) in the presence of IFN/RBV and one of the three iminosugar derivatives NB-DNJ (10, 50 or 100 μM), or N7-DGJ (100 μM), or NN-DNJ (50 μM). This allowed determination of both the length of time as well as the concentration of iminosugars needed for inclusion into the triple combination treatment, to prevent viral relapse when treatment was subsequently stopped. At the two triple combination treatment end-points chosen (passage 8 and passage 12, respectively), each sample was divided into three sets to allow the assessment of various follow-up treatment regimes: cells in set 1 continued to be cultured in the presence of the IFN/RBV/iminosugar triple cocktails; in set 2 all drugs were removed from the cells; and in set 3 only IFN/RBV were removed, i.e. the cells continued to be cultured in the presence of the iminosugar derivative only (iminosugar maintenance therapy). MTS-based cell proliferation assays confirmed that antiviral effects observed were not due to cytotoxicity, which was not significant for any of the drug combinations tested.

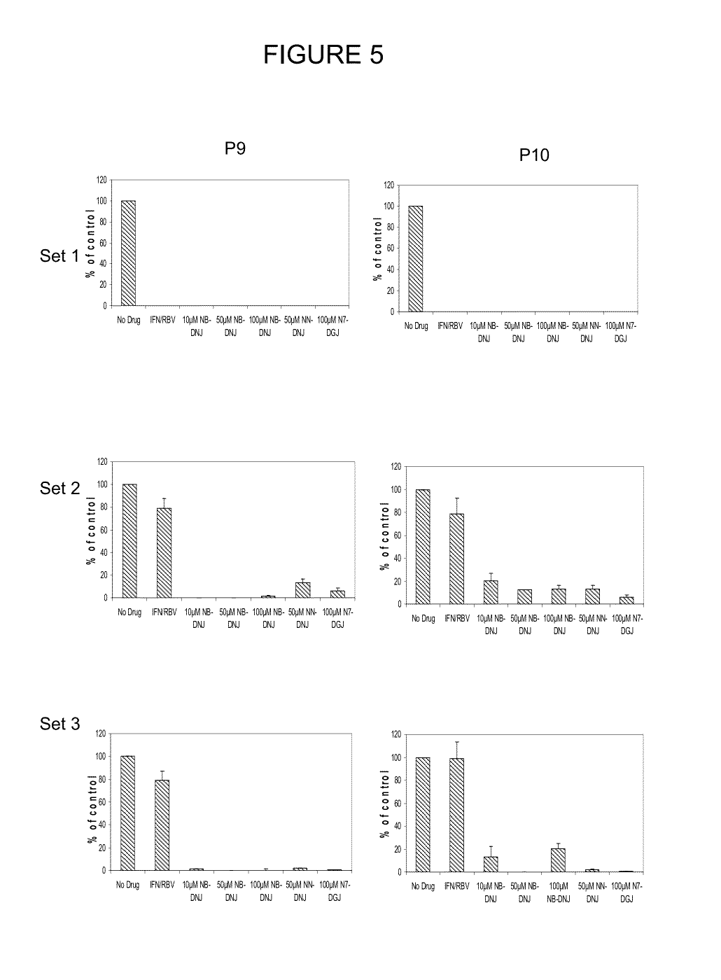

The supernatants of cells grown in the continuous presence of either the IFN/RBV double combination or any of the triple combinations including an iminosugar (Set 1), did not contain any viral RNA as measured by real time RT-PCR (FIG. 5, P9 and 10), nor any infectious virus as determined by infectivity studies using naïve MDBK cells in the two subsequent passages analysed.

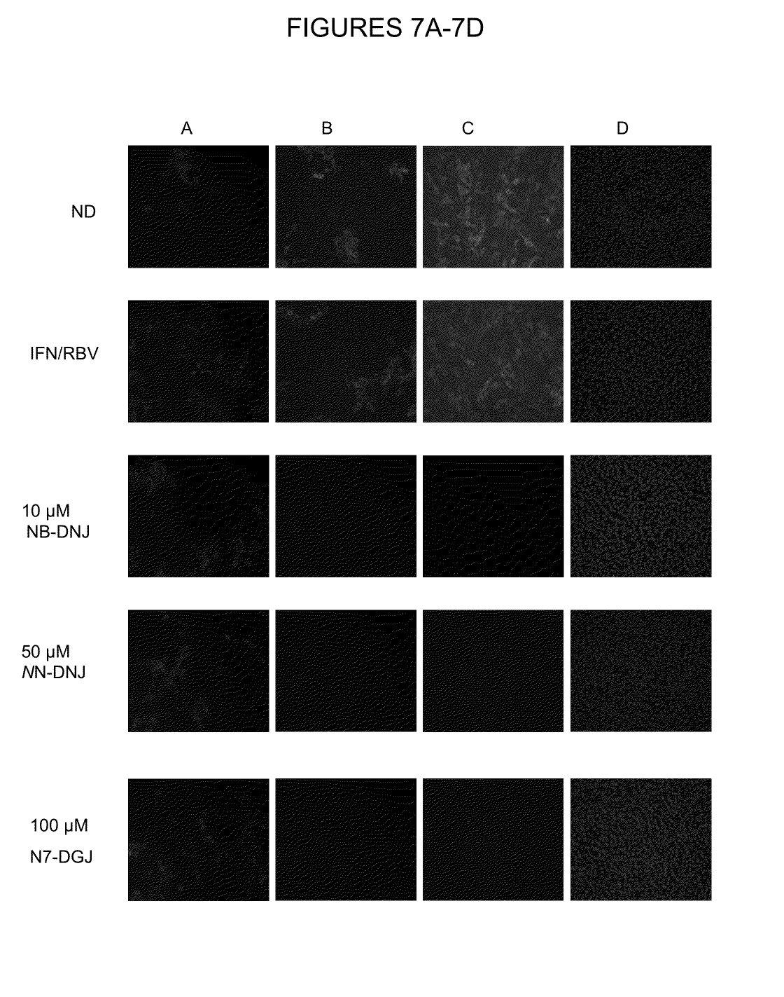

In contrast, when either all three drugs (Set 2) or IFN/RBV only (Set 3) were removed at passage 8 (the earlier of the two triple combination end-points), viral rebound was observed in all samples: In set 2, one passage after drug removal, viral rebound was immediate and most pronounced for samples that had been treated for 5 passages with IFN/RBV only. Viral RNA was also detected in samples treated with IFN/RBV in combination with 50 μM NN-DNJ or 100 μM N7-DGJ (FIG. 5, Set 2, P9). Viral rebound is delayed by one passage, to P10, for those samples that had been treated with IFN/RBV in combination with NB-DNJ (FIG. 5, Set 2, P10). Viral titres were higher in samples treated with IFN/RBV only compared to those treated with triple cocktails suggesting that iminosugars may be able to control or delay viral rebound after removal of the drugs (FIG. 5, set 2, P10). This observation is supported by results obtained with sample set 3 where, in the continued presence of an iminosugar, little or no viral RNA was detected one passage after the removal of IFN/RBV, though again rebound was only delayed and not entirely prevented (FIG. 5, Set 3, P9 and P10). In addition, infectious virus was detected as determined by infectivity studies using naïve MDBK cells for both Set 2 (FIG. 7) and Set 3 in the two subsequent passages analysed.

Combination Treatment of IFN/RBV with Iminosugars Eradicates BVDV Infection from a Persistently Infected MDBK Cell Line in a Time-Dependent Manner.

As combination therapy using IFN/RBV/iminosugar for five passages was not sufficient to prevent viral relapse after cessation of treatment, treatment with the triple combination was continued for a further 4 passages, i.e. these cells were treated with IFN/RBV or the various triple combinations for 9 passages (27 days) in total. At passage 12, samples were divided into three sets and the experiment repeated as before. This time, samples were monitored for a further 10 passages (P12-22; 30 days).

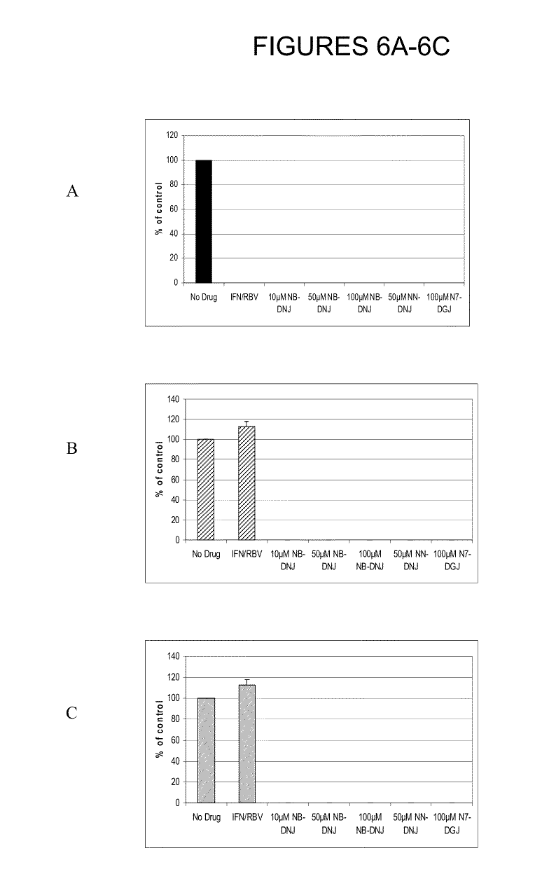

In the continued presence of all three drugs (Set 1) no viral RNA was detected by RT-PCR in any of the samples. By the conclusion of the experiment, the cells had been treated with IFN/RBV for 22 passages (or with triple cocktails for 19 passages) without any viral breakthrough occurring (FIG. 6A).

Surprisingly, even after the removal of all drugs (Set 2a) or INF/RBV (Set 3a), no viral rebound was detected as measured by RT-PCR in those samples that had been treated with an IFN/RBV/iminosugar triple cocktail, whereas viral rebound was detected in samples that had been treated with IFN/RBV only (FIGS. 6B and 6C). These data clearly indicate that inclusion of an iminosugar may be essential to eradicate BVDV infection entirely and prevent viral rebound. The RT-PCR data were confirmed by IF infectivity assays performed by infecting naïve cells with harvested supernatants. Infection was detected only in those samples that had not been drug treated at all, or had been treated with IFN/RBV alone (FIG. 7B). In addition, after the final passage (P22), the long term treated cells were also probed for the presence of BVDV and cells treated with IS were found to be negative (FIG. 7D). Together, these data indicate that extended treatment with any of the IFN/RBV/NB-DNJ, IFN/RBV/NN-DNJ or IFN/RBV/N7-DGJ triple cocktails may be able to clear the BVDV infection from a persistently infected MDBK cell line.

Treatment with IFN/RBV and NB-DNJ Eradicates BVDV Infection from a Persistently Infected MDBK Cell Line in a Dose-Dependent Manner and Prevents Viral Rebound.

After demonstrating the efficacy of iminosugars in combination with IFN and RBV, the minimum concentration of NB-DNJ required to eradicate BVDV infection from a persistently infected MDBK cell line was explored. The same cell culture experiments were performed with triple cocktails of IFN/RBV/NB-DNJ containing NB-DNJ at lower concentrations. Briefly, infected MDBK cells were cultured in the presence of IFN/RBV for three passages until the viral RNA signal dropped below detectable levels. Subsequently the medium was supplemented with 0.1, 1 or 10 μM NB-DNJ and the cells cultured for a further nine passages (P3-12) in the presence of the triple combinations. The cells were then divided into three sets as before and follow-up treatments were analysed by both RT-PCR and infectivity assays.

For infected cells that had been treated with IFN/RBV only, removal of these two drugs led to an immediate and pronounced viral rebound, the levels of which fluctuated (Table 5). After a large initial surge at P13, viral RNA levels dropped for several weeks, but at the final reading (P32) were higher than viral RNA levels in untreated controls. Similar fluctuations in viral RNA levels, which exceed those observed when establishing the stable infection in untreated naïve host cells at the start of the experiments, can be observed in all samples where rebound occurred.

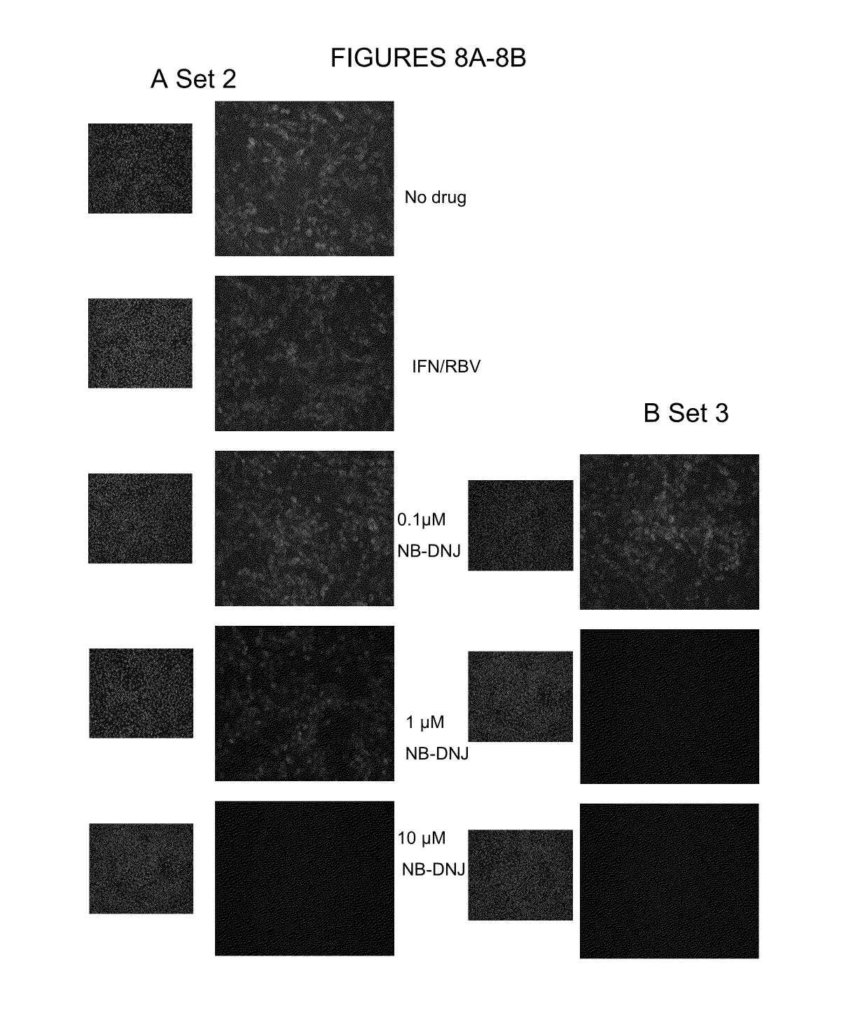

While in the continuous presence of drugs (Table 5, Set 1), no viral RNA could be detected, removal of all three drugs (Table 5, Set 2) resulted in viral rebound in those samples that had been treated with triple cocktails containing NB-DNJ at concentrations below 10 μM. Consistent with the previous experiment, neither viral RNA nor infectious virus was detected for those cells that had been treated for nine passages with IFN/RBV/10 μM NB-DNJ (Table 5). However, when treatment with NB-DNJ was maintained and only IFN/RBV was removed, no viral RNA or infectious virus was detected from those cells for which treatment with 1 or 10 μM NB-DNJ was maintained (Table 5, Set 3). Although delayed by four passages, viral rebound was observed under maintenance treatment with 0.1 μM NB-DNJ. These data indicate that inclusion of 10 μM NB-DNJ into the triple cocktail for the duration of nine passages is sufficient to permanently eradicate the virus even after withdrawal of all three drugs, whereas inclusion of 1 μM NB-DNJ into the initial triple cocktail requires continued maintenance with NB-DNJ after cessation of IFN/RBV treatment. The immunofluorescence analysis of the treated cells mirrored the real time PCR results and is shown in FIG. 8.

TABLE 5
Real time RT-PCR analysis of secreted viral RNA levels in the supernatants of cells
under various treatment regimes. RNA levels are expressed as percentage of those in non-
drug treated controls.

| | | Passage number | | | | | | | | | | | | | |
|---|---|---|---|---|---|---|---|---|---|---|---|---|---|---|---|
| | Treatment | P13 | P14 | P15 | P16 | P17 | P18 | P20 | P22 | P23 | P24 | P27 | P29 | P31 | P32 |
| Set 1 | No Drug | 100 | 100 | 100 | 100 | 100 | 100 | 100 | 100 | 100 | 100 | 100 | 100 | 100 | 100 |
| All drug on left | IFN/RBV | 0 | 0 | 0 | 0 | 0 | 0 | 0 | 0 | 0 | 0 | 0 | 0 | 0 | 0 |
| | 0.1 μM NB/DNJ | 0 | 0 | 0 | 0 | 0 | 0 | 0 | 0 | 0 | 0 | 0 | 0 | 0 | 0 |
| | 1 μM NB/DNJ | 0 | 0 | 0 | 0 | 0 | 0 | 0 | 0 | 0 | 0 | 0 | 0 | 0 | 0 |
| | 10 μM NB/DNJ | 0 | 0 | 0 | 0 | 0 | 0 | 0 | 0 | 0 | 0 | 0 | 0 | 0 | 0 |
| Set 2 | IFN/RBV | 0.7 | 967 | 17 | 42 | 21 | 31 | 21 | 118 | 84 | 80 | 86 | 138 | 90 | 130 |
| All drug | 0.1 μM NB/DNJ | 0 | 21 | 45 | 542 | 26 | 46 | 40 | 4 | 28 | 9 | 65 | 91 | 122 | 77 |
| removed | 1 μM NB/DNL | 0 | 0 | 84 | 106 | 6 | 23 | 23 | 136 | 10 | 9 | 78 | 163 | 151 | 86 |
| | 10 μM NB/DNJ | 0 | 0 | 0 | 0 | 0 | 0 | 0 | 0 | 0 | 0 | 0 | 0 | 0 | 0 |
| Set 3 | 0.1 μM NB/DNJ | 0 | 0 | 0 | 0 | 34 | 12 | 22 | 31 | 12 | 10 | 61 | 77 | 74 | 63 |
| IFN/RBV | 1 μM NB/DNJ | 0 | 0 | 0 | 0 | 0 | 0 | 0 | 0 | 0 | 0 | 0 | 0 | 0 | 0 |
| removed | 10 μM NB/DNJ | 0 | 0 | 0 | 0 | 0 | 0 | 0 | 0 | 0 | 0 | 0 | 0 | 0 | 0 |

### Discussion

Since the establishment of an HCV cell culture system ([9], [10], [11]), it has become possible to study most aspects of HCV morphogenesis and the HCV infection process. However, it is still not possible to consistently grow HCV in chronically infected cells for the length of time and at secreted viral RNA levels required to study long-term treatment with slower acting compounds, such as iminosugars, that target the morphogenesis process of the virus rather than viral RNA replication. In this respect BVDV, a close relative of HCV which supports the secretion of infectious virions in vitro, may be still the surrogate model of choice, especially since viral relapse after IFN/RBV treatment, an event frequently observed after cessation of anti-HCV therapy, [12], [13], is mirrored in the BVDV/MDBK system (Table 5) but has not yet been reported in the HCV cell culture infectivity system. The BVDV model system was used to show that the addition of morphogenesis inhibitors to IFN/RBV has the potential to eradicate virus from persistently infected cells and to prevent viral relapse after treatment is stopped. Supplementing current standard of care drugs with compounds, such as iminosugars, which target entirely different steps in the viral life cycle, is sufficient to cope with those undetectable (by real time RT-PCR and infectivity assays) yet undisputedly remaining viruses that lead to the quick and frequently strong viral re-occurrence and expansion after IFN and RBV are removed. DNJ-containing iminosugars cause misfolding of the viral envelope glycoproteins (via ER alpha-glucosidase inhibition) and subsequent impairment of viral secretion and infectivity, as demonstrated for BVDV [14] [6] and HCV [15]. In addition, long alkylchain containing iminosugars (such as NN-DNJ, NN-DGJ and N7-DGJ) inhibit the viral ion channel p7 [4] [15], which may be crucial for the secretion of infectious virus for both BVDV [16] and HCV) [17], [18] [19]. Long alkyl chain carrying DNJ compounds may employ both mechanisms of action.

The present study was mainly focussed on the short alkyl chain carrying ER-alpha glucosidase inhibitor NB-DNJ, as this compound has a history of safe use [20] [21]. In the BVDV/MDBK system, inclusion of 10 μM NB-DNJ successfully prevents relapse after cessation of triple therapy and 1 μM NB-DNJ is sufficient to prevent relapse when administered continuously as monotherapy during maintenance treatment after removal of IFN/RBV. Such concentration range can be achieved and tolerated in human patients [20]. Although the invention is not limited by its theoty of operation, it may proposed that the likelihood of viruses to accumulate mutations, which could enable them to become independent of either iminosugar target (the host cell encoded ER alpha glucosidases or the p7 ion channel), is much reduced compared to the demonstrated speed of viral escape mutants emerging in the presence of inhibitors targeting virally encoded enzymes, such as the polymerase or protease [22] [23].

Ouzounov et al. have reported that higher NB-DNJ concentrations in combination with IFN show a greater than additive antiviral effect in an experimental setting using a MOI of >1 analysing a single replication cycle of cytopathic BVDV[24]. The non-cytopathic BVDV system used in the present study mirrors a chronic viral infection, such as HCV, with a lower MOI over several viral and cellular replication cycles. In this system, no synergy was observed when physiologically achievable NB-DNJ concentrations were added to a high concentration IFN/RBV combination, i.e. when iminosugars were added to IFN/RBV at the start of the treatment no additional beneficial effect was observed during the three passages needed to reduce the viral signal below detection limits. Therefore, the iminosugars were added after the initial IFN/RBV induced strong decrease in viral RNA levels instead, when the potentially available mutant pool is smallest. Using such treatment protocol, each of the iminosugars tested showed efficacy and potential to eradicate persistent BVDV infection from MDBK cells with prevention of viral relapse after cessation of treatment. For NB-DNJ, it was shown that the eradication was time and dose dependent.

Significantly, because of the targets involved, all HCV genotypes, including the challenging genotype 1 responsible for most cases of viral relapse observed in human patients [25] [26], may be predicted to respond to iminosugar treatment.

### REFERENCES LIST

1. Buckwold, V. E., et al. Antiviral Res, 2003. 60(1): p. 1-15.
2. National Health Service, Treatment for Hepatitis C. 2007 [cited 2007 Jun. 8, 2007]; Available from: http://www.hepc.nhs.uk/professionals/treatment.html.
3. Henzler, H. J. and K. Kaiser, Nat Biotechnol, 1998. 16(11): p. 1077-9.
4. Pavlovic, D., et al. Proc Natl Acad Sci USA, 2003. 100(10): p. 6104-8.
5. Durantel, D., et al., Antimicrob Agents Chemother, 2004. 48(2): p. 497-504.
6. Durantel, D., et al., J Virol, 2001. 75(19): p. 8987-98.
7. Branza-Nichita, N., et al. Biochem Biophys Res Commun, 2004. 319(2): p. 655-62.
8. Zitzmann, N., et al., Proc Natl Acad Sci USA, 1999. 96(21): p. 11878-82.
9. Lindenbach, B. D., et al., Science, 2005. 309(5734): p. 623-6.
10. Wakita, T., et al., Nat Med, 2005. 11(7): p. 791-6.
11. Zhong, J., et al., Proc Natl Acad Sci USA, 2005. 102(26): p. 9294-9.
12. Poynard, T., et al., Lancet, 2003. 362(9401): p. 2095-100.
13. Basso, M., et al., Dig Liver Dis, 2007. 39(1): p. 47-51.
14. Branza-Nichita, N., et al., J Virol, 2001. 75(8): p. 3527-36.
15. Steinmann, E., et al., Hepatology, 2007. 46(2): p. 330-338.
16. Harada, T., N. Tautz, and H. J. Thiel, J Virol, 2000. 74(20): p. 9498-506.
17. Steinmann, E., et al., PLoS Pathog, 2007. 3(7): p. e103.
18. Sakai, A., et al., Proc Natl Acad Sci USA, 2003. 100(20): p. 11646-51.
19. Jones, C. T., et al., J Virol, 2007. 81(16): p. 8374-83.
20. Cox, T., et al., Lancet, 2000. 355(9214): p. 1481-5.
21. Patterson, M. C., et al., Lancet Neurol, 2007.
22. Courcambeck, J., et al., Antivir Ther, 2006. 11(7): p. 847-55.
23. Neyts, J. Antiviral Res, 2006. 71(2-3): p. 363-71.
24. Ouzounov, S., et al., Antiviral Res, 2002. 55(3): p. 425-35.
25. Manns, M. P., et al., Lancet, 2001. 358(9286): p. 958-65.
26. Hadziyannis, S. J., et al., Ann Intern Med, 2004. 140(5): p. 346-55.

### Additional Embodiments

1. A method comprising: contacting a mammalian cell infected with a virus with(a) a first compound, wherein the first compound is a compound of Formula I, a pharmaceutically acceptable salt thereof, or a mixture of any two or more thereof:

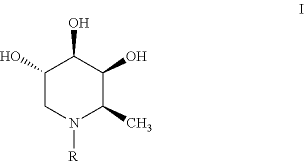

wherein R is selected from substituted or unsubstituted alkyl groups, and substituted or unsubstituted oxaalkyl groups; and
(b) at least one of a second compound and a third compound;
wherein the second compound is selected from a nucleotide antiviral compound, a nucleoside antiviral compound, or a mixture of any two or more thereof; and
wherein the third compound is selected from an immunostimulating compound, an immunomodulating compound, or a mixture of any two or more thereof;
wherein the first compound, the second compound, and the third compound are contacted in an amount effective to inhibit the virus.
   2. The method of embodiment 1, wherein R is a substituted or unsubstituted alkyl group, or a substituted or unsubstituted oxaalkyl group having from 1 to 16 carbon atoms.
   3. The method of embodiment 1, wherein R is a substituted or unsubstituted alkyl group, or a substituted or unsubstituted oxaalkyl group having from 4 to 12 carbon atoms.
   4. The method of embodiment 1, wherein R is a substituted or unsubstituted alkyl group, or a substituted or unsubstituted oxaalkyl group having 8 to 10 carbon atoms.
   5. The method of embodiment 1, wherein R is a substituted or unsubstituted alkyl group, or a substituted or unsubstituted oxaalkyl group having from 1 to 16 carbon atoms and from 1 to 4 oxygen atoms.
   6. The method of embodiment 1, wherein R is a substituted or unsubstituted alkyl group, or a substituted or unsubstituted oxaalkyl group having from 4 to 12 carbon atoms and from 1 to 2 oxygen atoms.
   7. The method of embodiment 5, wherein R is —(CH₂)₆OCH₂CH₃.
   8. The method of embodiment 1, wherein the nucleotide antiviral compound is selected from purine nucleotide antiviral compounds, pyrimidine nucleotide antiviral compounds, or a mixture of any two or more thereof; and the nucleoside antiviral compound is selected from purine nucleoside antiviral compounds, pyrimidine nucleoside antiviral compounds, or a mixture of any two or more thereof.
   9. The method of embodiment 8, wherein the second compound is 1-beta-D-ribafuranosyl-1,2,4-triazole-3-carboxamide.
   10. The method of embodiment 1, wherein the third compound is an interferon.
   11. The method of embodiment 10, wherein the third compound is selected from a group consisting of alpha interferons, beta interferons, pegylated alpha interferons, pegylated beta interferons, and mixtures of any two or more thereof.
   12. The method of embodiment 1, wherein R is —(CH₂)₆OCH₂CH₃; the second compound comprises 1-β-D-ribofuranosyl-1,2,4-triazole-3-carboxamide; and the third compound comprises interferon.
   13. The method of embodiment 1, wherein the mammalian cell is a human cell.
   14. The method of embodiment 1, wherein the contacting a mammalian cell comprises administering the first compound, the second compound, and the third compound to a mammal.
   15. The method of embodiment 14, wherein the mammal is a human.
   16. The method of embodiment 14, wherein the first compound, the second compound, and the third compound are administered to the mammal separately, sequentially, or simultaneously.
   17. The method of embodiment 1, wherein the virus is a flavivirus or a hepadnavirus.
   18. The method of embodiment 1, wherein the virus is a hepatitis virus and the amount effective to inhibit the virus is an amount effective to inhibit the hepatitis virus.
   19. The method of embodiment 18, wherein the hepatitis virus is a hepatitis B virus.
   20. The method of embodiment 18, wherein the hepatitis virus is a hepatitis C virus.
   21. The method of embodiment 18, wherein said hepatitis virus is a bovine viral diarrhea virus.
   22. A kit comprising:
(a) a first compound, wherein the first compound is a compound of Formula I, a pharmaceutically acceptable salt thereof, or a mixture of any two or more thereof:

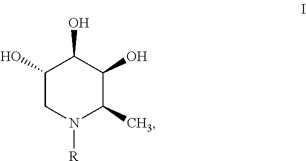

wherein R is selected from substituted or unsubstituted alkyl groups, or substituted or unsubstituted oxaalkyl groups, and;
(b) at least one of a second compound and a third compound, wherein
wherein the second compound is selected from a nucleotide antiviral compound, a nucleoside antiviral compound, or a mixture of any two or more thereof; and
wherein the third compound is selected from an immunostimulating compound, an immunomodulating compound, or a mixture of any two or more thereof;
wherein the first compound, the second compound, and the third compound are in an amount effective to inhibit a virus infecting a mammal.
   23. The kit of embodiment 22, wherein the virus is a flavivirus or a hepadnavirus.
   24. The kit of embodiment 22, wherein R is a substituted or unsubstituted alkyl group, or a substituted or unsubstituted oxaalkyl group having from 1 to 16 carbon atoms, and from 1 to 4 oxygen atoms.
   25. The kit of embodiment 22, wherein the nucleotide antiviral compound is selected from purine nucleotide antiviral compounds, pyrimidine nucleotide antiviral compounds, or a mixture of any two or more thereof; and the nucleoside antiviral compound is selected from purine nucleoside antiviral compounds, pyrimidine nucleoside antiviral compounds, or a mixture of any two or more thereof. 26. The kit of embodiment 25, wherein the second compound is 1-beta-D-ribafuranosyl-1,2,4-triazole-3-carboxamide.
   27. The kit of embodiment 22, wherein the third compound is an interferon.
   28. The kit of embodiment 22, wherein the third compound is selected from a group consisting of alpha interferons, beta interferons, pegylated alpha interferons, pegylated beta interferons, and mixtures of any two or more thereof.
   29. The kit of embodiment 22, wherein R is —(CH₂)₆OCH₂CH₃; the second compound comprises 1-β-D-ribofuranosyl-1,2,4-triazole-3-carboxamide; and the third compound comprises interferon.
   30. The kit of embodiment 22, wherein the first compound, the second compound, and the third compound form a pharmaceutical composition for simultaneous administration to the mammal.
   31. The kit of embodiment 22, wherein the first compound, the second compound, and the third compound are for separate or sequential administration to the mammal.
   32. The kit of embodiment 22, wherein the second compound and third compound comprise a single composition.
   33. A composition comprising:
(a) a first compound, wherein the first compound is a compound of Formula I, a pharmaceutically acceptable salt thereof, or a mixture of any two or more thereof:

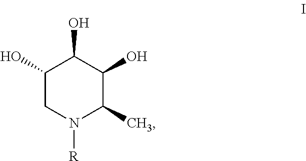

wherein R is selected from substituted or unsubstituted alkyl groups, or substituted or unsubstituted oxaalkyl groups; and
(b) at least one compound selected from a second compound and a third compound, wherein the second compound is a nucleotide antiviral compound, a nucleoside antiviral compound, or a mixture of any two or more thereof; and
wherein the third compound is selected from an immunostimulating compound, an immunomodulating compound, or a mixture of any two or more thereof;
wherein the first compound, the second compound, and the third compound are in an amount effective to inhibit a virus infecting a mammal.
   34. The composition of embodiment 33, further comprising a pharmaceutically acceptable carrier.
   35. The composition of embodiment 33, wherein R is —(CH₂)₆OCH₂CH₃; the second compound comprises 1-β-D-ribofuranosyl-1,2,4-triazole-3-carboxamide; and the third compound comprises interferon.
   36. A method of treating or preventing a viral infection, comprising
administering to a subject in need thereof a combination that comprises a) an immunostimulating or immunomodulating agent and b) a nucleotide or a nucleoside antiviral agent, provided that said combination does not inhibit a host enzyme or does not an ion channel activity; and then
after a period of time sufficient to permit the combination to enhance activity of a second administration step, administering to the subject the combination and a compound that is at least one of a host enzyme inhibitor or an ion channel inhibitor.
   37. The method of embodiment 36, further comprising
withdrawing administering the combination after administering the combination and the compound for a period of time sufficient to treat the viral infection, wherein at least 3 days after the withdrawing no rebound of the viral infection occurs in the subject.
   38. The method of embodiment 37, wherein at least 10 days after the withdrawing no rebound of the viral infection occurs in the subject.
   39. The method of embodiment 38, wherein at least 30 days after the withdrawing no rebound of the viral infection occurs in the subject.
   40. The method of embodiment 37, wherein the withdrawing comprises withdrawing administering the compound.
   41. The method of embodiment 36, wherein the compound is an iminosugar.
   42. The method of embodiment 36, wherein the compound is a host enzyme inhibitor.
   43. The method of embodiment 42, wherein the compound is a glucosidase inhibitor.
   44. The method of embodiment 36, wherein the compound is an ion channel activity inhibitor.
   45. The method of embodiment 36, wherein the compound is both a host enzyme inhibitor and an ion channel activity inhibitor.
   45. The method of embodiment 36, wherein the compound has Formula I:

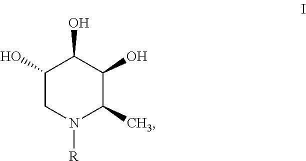

wherein R is selected from substituted or unsubstituted alkyl groups, and substituted or unsubstituted oxaalkyl groups.
   46. The method of embodiment 45, wherein R is a substituted or unsubstituted alkyl group or a substituted or unsubstituted oxaalkyl group having from 1 to 16 carbon atoms.
   47. The method of embodiment 46, wherein R is —(CH₂)₆OCH₂CH₃.
   48. The method of embodiment 36, wherein the compound has Formula II

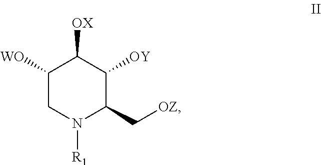

wherein R₁ is selected from substituted or unsubstituted alkyl groups, substituted or unsubstituted cycloalkyl groups, substituted or unsubstituted aryl groups, or substituted or unsubstituted oxaalkyl groups and wherein W, X, Y, and Z are each independently selected from hydrogen, alkanoyl groups, aroyl groups, and haloalkanoyl groups.
   49. The method of embodiment 48, wherein R₁ is a substituted or unsubstituted alkyl group having from 1 to 16 carbon atoms and W, X, Y and Z are each hydrogen.
   50. The method of embodiment 49, wherein R₁ has from 2 to 6 carbon atoms.
   51. The method of embodiment 50, wherein R₁ is butyl.
   52. The method of embodiment 49, wherein R₁ has from 7 to 12 carbon atoms.
   53. The method of embodiment 52, wherein R₁ is nonyl.
   54. The method of embodiment 36, wherein the nucleotide antiviral agent is selected from purine nucleotide antiviral agents, pyrimidine nucleotide antiviral agents, or a mixture of any two or more thereof; and the nucleoside antiviral agent is selected from purine nucleoside antiviral agents, pyrimidine nucleoside antiviral agents, or a mixture of any two or more thereof.
   55. The method of embodiment 54, wherein the nucleotide antiviral agent is 1-beta-D-ribafuranosyl-1,2,4-triazole-3-carboxamide.
   56. The method of embodiment 36, wherein the immunostimulating agent is an interferon.
   57. The method of embodiment 56, wherein the interferon is selected from the group consisting of alpha interferons, beta interferons, pegylated alpha interferons, pegylated beta interferons, and mixtures of any two or more thereof.
   58. The method of embodiment 36, wherein the immunostimulating agent is interferon and the nucleotide antiviral agent is 1-beta-D-ribafuranosyl-1,2,4-triazole-3-carboxamide.
   59. The method of embodiment 36, wherein the infection is an infection caused by or associated with a virus belonging to the Flaviridae family.
   60. The method of embodiment 36, wherein the infection is a Hepatitis infection.
   61. The method of embodiment 36, wherein the infection is an infection caused by or associated with a BVDV virus.
   62. The method of embodiment 36, wherein the infection is a Hepatitis C infection.
   63. The method of embodiment 36, wherein the infection is a Hepatitis C infection, the immunostimulating agent is interferon, the nucleotide antiviral agent is 1-beta-D-ribafuranosyl-1,2,4-triazole-3-carboxamide and the compound is selected from N-butyl deoxynojirimycin, N-nonyl deoxynojirimycin and N-(7-oxa-nonyl)-1,5,6-trideoxy-1,5-imino-D-galactitol.
   64. The method of embodiment 36, wherein the subject is a mammal.
   65. The method of embodiment 64, wherein the subject is a human.
   66. A method of treating or preventing a viral infection, comprising
decreasing a level of the viral infection in a subject in need thereof by first administering to the subject a pharmaceutical composition that does not inhibit a host enzyme or does not inhibit an ion channel activity;
administering to the subject the composition, and a compound that is at least one of a host enzyme inhibitor or an ion channel inhibitor.
   67. The method of embodiment 66, wherein said decreasing comprises decreasing the level of the viral infection to a non-detectable level.
   68. The method of embodiment 66, wherein the composition comprises at least one of an immunostimulating or immunomodulating agent and an nucleoside or nucleotide agent.
   69. The method of embodiment 68, further comprising
withdrawing administering the composition after administering the composition and the compound for a period of time sufficient to treat the infection, wherein at least 3 days after the withdrawing no rebound of the viral infection occurs in the subject.
   70. The method of embodiment 69, wherein at least 10 days after the withdrawing no rebound of the viral infection occurs in the subject.
   71. The method of embodiment 70, wherein at least 30 days after the withdrawing no rebound of the viral infection occurs in the subject.
   72. The method of embodiment 66, wherein the withdrawing comprises withdrawing administering the compound.
   73. The method of embodiment 66, wherein the compound is an iminosugar.
   74. The method of embodiment 66, wherein the compound is a host enzyme inhibitor.
   75. The method of embodiment 74, wherein the compound is a glucosidase inhibitor.
   76. The method of embodiment 66, wherein the compound is an ion channel activity inhibitor.
   77. The method of embodiment 66, wherein the compound is both a host enzyme inhibitor and an ion channel inhibitor.
   78. The method of embodiment 64, wherein the compound has Formula I:

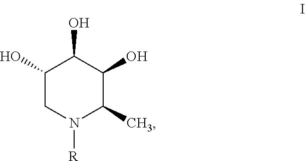

wherein R is selected from substituted or unsubstituted alkyl groups, and substituted or unsubstituted oxaalkyl groups.
   79. The method of embodiment 72, wherein R is a substituted or unsubstituted alkyl group or a substituted or unsubstituted oxaalkyl group having from 1 to 16 carbon atoms.
   80. The method of embodiment 73, wherein R is —(CH₂)₆OCH₂CH₃.
   81. The method of embodiment 66, wherein the compound has Formula II

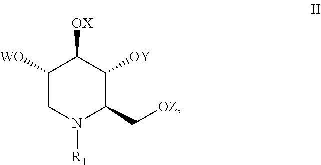

wherein R₁ is selected from substituted or unsubstituted alkyl groups, substituted or unsubstituted cycloalkyl groups, substituted or unsubstituted aryl groups, or substituted or unsubstituted oxaalkyl groups and wherein W, X, Y, and Z are each independently selected from hydrogen, alkanoyl groups, aroyl groups, and haloalkanoyl groups.
   82. The method of embodiment 81, wherein R₁ is a substituted or unsubstituted alkyl group having from 1 to 16 carbon atoms and W, X, Y and Z are each hydrogen.
   83. The method of embodiment 82, wherein R₁ has from 2 to 6 carbon atoms.
   84. The method of embodiment 83, wherein R₁ is butyl.
   85. The method of embodiment 82, wherein R₁ has from 7 to 12 carbon atoms.
   86. The method of embodiment 85, wherein R₁ is nonyl.
   87. The method of embodiment 66, wherein the nucleotide antiviral agent is selected from purine nucleotide antiviral agents, pyrimidine nucleotide antiviral agents, or a mixture of any two or more thereof; and the nucleoside antiviral agent is selected from purine nucleoside antiviral agents, pyrimidine nucleoside antiviral agents, or a mixture of any two or more thereof.
   88. The method of embodiment 87, wherein the nucleotide antiviral agents 1-beta-D-ribafuranosyl-1,2,4-triazole-3-carboxamide.
   89. The method of embodiment 66, wherein the immunostimulating agent is an interferon.
   90. The method of embodiment 89, wherein the interferon is selected from a group consisting of alpha interferons, beta interferons, pegylated alpha interferons, pegylated beta interferons, and mixtures of any two or more thereof.
   91. The method of embodiment 66, wherein the immunostimulating agent is interferon and the nucleotide antiviral agents 1-beta-D-ribafuranosyl-1,2,4-triazole-3-carboxamide.
   92. The method of embodiment 66, wherein the viral infection is an infection caused by or associated with a virus belonging to the Flaviridae family.
   93. The method of embodiment 66, wherein the infection is a Hepatitis C infection.
   94. The method of embodiment 66, wherein the infection is a Hepatitis C infection, the immunostimulating agent is interferon, the nucleotide antiviral agent is 1-beta-D-ribafuranosyl-1,2,4-triazole-3-carboxamide and the iminosugar is selected from N-butyl deoxynojirimycin, N-nonyl deoxynojirimycin and N-(7-oxa-nonyl)-1,5,6-trideoxy-1,5-imino-D-galactitol.
   95. The method of embodiment 66, wherein the subject is a mammal.
   96. The method of embodiment 91, wherein the subject is a human.

What is claimed is:

1. A method of preventing a relapse of Hepatitis C infection comprising:
administering for a second time period to a subject in need thereof at least one first antiviral agent and at least one second antiviral agent, wherein: a) said at least one first antiviral agent does not inhibit host α-glucosidase, b) said at least one second antiviral agent inhibits host α-glucosidase and c) said subject exhibited a negative Hepatitis C viral load upon administering the at least one first antiviral agent, but not the at least one second antiviral agent for a first time period prior to the start of said second period.

2. The method of claim 1, wherein said first antiviral agent comprises at least one nucleotide or nucleoside antiviral agent and an interferon receptor antagonist.

3. The method of claim 2, wherein said interferon receptor antagonist is interferon.

4. The method of claim 3, wherein said interferon is alpha interferon.

5. The method of claim 4, wherein said alpha interferon is a pegylated alpha interferon.

6. The method of claim 2, wherein said at least one nucleotide or nucleoside antiviral agent comprises ribavirin or a derivative thereof.

7. The method of claim 2, wherein the at least one second antiviral agent comprises a compound of formula II or a pharmaceutically acceptable salt thereof: 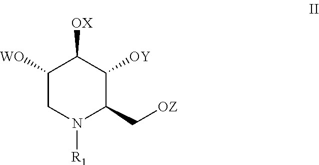
wherein R1 is selected from substituted or unsubstituted alkyl groups, substituted or unsubstituted cycloalkyl groups, substituted or unsubstituted aryl groups, or substituted or unsubstituted oxaalkyl groups and wherein W, X, Y, and Z are each independently selected from hydrogen, alkanoyl groups, aroyl groups, and haloalkanoyl groups.

8. The method of claim 7, wherein R1 is a substituted or unsubstituted alkyl group having from 1 to 16 carbon atoms and W, X, Y and Z are each hydrogen.

9. The method of claim 8, wherein R1 has from 2 to 6 carbon atoms.

10. The method of claim 9, wherein R1 is butyl.

11. The method of claim 1, wherein the at least first antiviral agent comprises ribavirin and interferon and the at least one second antiviral agent comprises N-butyl deoxynojirimycin.

12. The method of claim 1, further comprising after the second time period, administering the at least one second antiviral agent for a third time period, while withdrawing administering the at least one first antiviral agent.

13. The method of claim 1, wherein a subject is a human.

14. A method of preventing a relapse of Hepatitis C infection comprising:
administering for a second time period to a subject in need thereof at least one first antiviral agent and at least one second antiviral agent, wherein: a) said at least one first antiviral agent does not comprise an iminosugar, b) said at least one second antiviral agent comprises an iminosugar and c) said subject exhibited a negative Hepatitis C viral load upon administering the at least one first antiviral agent, but not the at least one second antiviral agent for a first time period prior to the start of said second period.

15. The method of claim 14, wherein said first antiviral agent comprises at least one nucleotide or nucleoside antiviral agent and an interferon receptor antagonist.

16. The method of claim 15, wherein said interferon receptor antagonist is interferon.

17. The method of claim 16, wherein said interferon is alpha interferon.

18. The method of claim 17, wherein said alpha interferon is a pegylated alpha interferon.

19. The method of claim 15, wherein said at least one nucleotide or nucleoside antiviral agent comprises ribavirin or a derivative thereof.

20. The method of claim 15, wherein the iminosugar is a compound of formula II or a pharmaceutically acceptable salt thereof: 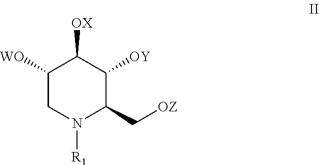
wherein R₁ is selected from substituted or unsubstituted alkyl groups, substituted or unsubstituted cycloalkyl groups, substituted or unsubstituted aryl groups, or substituted or unsubstituted oxaalkyl groups and wherein W, X, Y, and Z are each independently selected from hydrogen, alkanoyl groups, aroyl groups, and haloalkanoyl groups.

21. The method of claim 20, wherein R1 is a substituted or unsubstituted alkyl group having from 1 to 16 carbon atoms and W, X, Y and Z are each hydrogen.

22. The method of claim 21, wherein R1 has from 2 to 6 carbon atoms.

23. The method of claim 22, wherein R1 is butyl.

24. The method of claim 14, wherein the at least first antiviral agent comprises ribavirin and interferon and the at least one second antiviral agent comprises N-butyl deoxynojirimycin.

25. The method of claim 14, further comprising after the second time period, administering the at least one second antiviral agent for a third time period, while withdrawing administering the at least one first antiviral agent.

26. The method of claim 14, wherein a subject is a human.
